(19) United States
(12) Patent Application Publication
(10) Pub. No.: US 2024/0293415 A1
(43) Pub. Date: Sep. 5, 2024

(54) COMBINATION THERAPIES

(71) Applicant: Infinity Pharmaceuticals, Inc., Cambridge, MA (US)

(72) Inventor: Jeffery L. KUTOK, Natick, MA (US)

(21) Appl. No.: 18/644,709

(22) Filed: Apr. 24, 2024

Related U.S. Application Data
(63) Continuation of application No. 17/474,282, filed on Sep. 14, 2021.
Application No. 17/474,282 is a continuation of application No. 16/312,896, filed on Dec. 21, 2018, now Pat. No. 11,147,818.
(60) Provisional application No. 62/354,637, filed on Jun. 24, 2016.

(51) Int. Cl. A61K 31/52; A61K 31/497; A61K 39/00; A61K 39/395; A61P 35/00; C07K 16/28

(52) U.S. Cl. CPC A61K 31/52; A61K 31/497; A61K 39/39566; A61P 35/00; C07K 16/2803; C07K 16/2818; A61K 2039/505; C07K 2317/21; C07K 2317/24

(57) ABSTRACT

Provided herein are pharmaceutical compositions comprising a phosphatidylinositol 3-kinase inhibitor, or pharmaceutically acceptable form thereof, in combination with a second agent, or a pharmaceutically acceptable form thereof, wherein the second agent is chosen from one or more of 1) a checkpoint modulator, 2) an XPO1 inhibitor, 3) an anti-CD19 antibody, 4) a TLR agonist, 5) a STING agonist, or 6) a Flt3 ligand, or a combination thereof. Also provided herein are methods of treatment comprising administration of the compositions, and uses of the compositions, e.g., for treatment of cancer.

This application claims priority to U.S. Provisional Application No. 62/354,637, filed Jun. 24, 2016, the entirety of which is incorporated herein by reference.

### BACKGROUND

The phosphoinositide 3-kinases (PI3Ks) signaling pathway is one of the most highly mutated systems in human cancers. PI3Ks are members of a unique and conserved family of intracellular lipid kinases that phosphorylate the 3′—OH group on phosphatidylinositols or phosphoinositides. The PI3K family comprises 15 kinases with distinct substrate specificities, expression patterns, and modes of regulation. The class I PI3Ks (p110α, p1101β, p110δ, and p110γ) are typically activated by tyrosine kinases or G-protein coupled receptors to generate phosphatidylinositol (3,4,5)-trisphosphate (PIP3), which engages downstream effectors such as those in the AKT/PDK1 pathway, mTOR, the Tec family kinases, and the Rho family GTPases. The class II and III PI3Ks play a key role in intracellular trafficking through the synthesis of phosphatidylinositol 3-bisphosphate (PI(3)P) and phosphatidylinositol (3,4)-bisphosphate (PI(3,4)P2). The PI3Ks are protein kinases that control cell growth (mTORC1) or monitor genomic integrity (ATM, ATR, DNA-PK, and hSmg-1).

There are four mammalian isoforms of class I PI3Ks: PI3K-α, β, δ (class Ia PI3Ks) and PI3K-γ (a class 1b PI3K). These enzymes catalyze the production of PIP3, leading to activation of downstream effector pathways important for cellular survival, differentiation, and function. PI3K-α and PI3K-β are widely expressed and are important mediators of signaling from cell surface receptors. PI3K-α is the isoform most often found mutated in cancers and has a role in insulin signaling and glucose homeostasis (Knight et al. Cell (2006) 125(4):733-47; Vanhaesebroeck et al. Current Topic Microbiol. Immunol. (2010) 347:1-19). PI3K-β is activated in cancers where phosphatase and tensin homolog (PTEN) is deleted. Both isoforms are targets of small molecule therapeutics in development for cancer.

PI3K-δ and-γ are preferentially expressed in leukocytes and are important in leukocyte function. These isoforms also contribute to the development and maintenance of hematologic malignancies (Vanhaesebroeck et al. Current Topic Microbiol. Immunol. (2010) 347:1-19; Clayton et al. J Exp Med. (2002) 196(6):753-63; Fung-Leung Cell Signal. (2011) 23(4):603-8; Okkenhaug et al. Science (2002) 297(5583):1031-34). PI3K-δ is activated by cellular receptors (e.g., receptor tyrosine kinases) through interaction with the Sarc homology 2 (SH2) domains of the PI3K regulatory subunit (p85), or through direct interaction with RAS.

### SUMMARY

Provided herein are, at least in part, compositions and methods comprising a PI3K inhibitor in combination with a selected second therapeutic agent. In one embodiment, it has been discovered that combinations of a PI3K inhibitor with a second therapeutic agent chosen from one or more of: 1) a checkpoint modulator, 2) an XPO1 inhibitor, 3) an anti-CD19 antibody, 4) a TLR agonist, 5) a STING agonist, or 6) a Flt3 ligand, or a combination thereof, have a synergistic effect in treating a cancer (e.g., in reducing cancer cell growth or viability, or both). The combinations of PI3K inhibitors and selected second therapeutic agents can allow the PI3K inhibitor, the second therapeutic agent, or both, to be administered at a lower dosage than would be required to achieve the same therapeutic effect compared to a monotherapy dose. In some embodiments, the combination can allow the PI3K inhibitor, the second therapeutic agent, or both, to be administered at a lower frequency than if the PI3K inhibitor or the second therapeutic agent were administered as a monotherapy. Such combinations can provide advantageous effects, e.g., in reducing, preventing, delaying, and/or decreasing in the occurrence of one or more of: a side effect, toxicity, or resistance that would otherwise be associated with administration of a higher dose of the agents.

Accordingly, in one aspect, provided herein is a composition (e.g., one or more pharmaceutical compositions or dosage forms), comprising a PI3K inhibitor (e.g., one or more PI3K inhibitors), or a pharmaceutically acceptable form thereof, in combination with a second agent (e.g., one or more second therapeutic agents), or a pharmaceutically acceptable form thereof. In certain embodiments, the second therapeutic agent is chosen from one or more of: 1) a checkpoint modulator, 2) an XPO1 inhibitor, 3) an anti-CD19 antibody, 4) a TLR agonist, 5) a STING agonist, or 6) a Flt3 ligand, or a combination thereof. The PI3K inhibitor and the second agent can be present in a single composition or as two or more different compositions. The PI3K inhibitor and the second agent can be administered via the same administration route or via different administration routes.

In some embodiments, the composition (e.g., one or more compositions or dosage forms) comprising the combination of PI3K inhibitor and the second agent is synergistic, e.g., has a synergistic effect in treating a cancer (e.g., in reducing cancer cell growth or viability, or both). In certain embodiments, the amount or dosage of the PI3K inhibitor, the second agent, or both, present in the composition(s) does not exceed the level at which each agent is used individually, e.g., as a monotherapy. In certain embodiments, the amount or dosage of the PI3K inhibitor, the second agent, or both, present in the composition(s) is lower (e.g., at least 20%, at least 30%, at least 40%, or at least 50%) than the amount or dosage of each agent used individually, e.g., as a monotherapy. In other embodiments, the amount or dosage of the PI3K inhibitor, the second agent, or both, present in the composition(s) that results in a desired effect (e.g., treatment of cancer, achieve inhibition (e.g., 50% inhibition), achieve growth inhibition (e.g., 50% growth inhibition), or achieve a therapeutic effect) is lower (e.g., at least 20%, at least 30%, at least 40%, or at least 50% lower) than the amount or dosage of each agent used individually, e.g., as a monotherapy. In certain embodiments, the frequency of administration of the PI3K inhibitor that achieves a therapeutic effect is lower (e.g., at least 20%, 30%, 40%, or 50% lower), when the PI3K inhibitor is administered in combination with the second agent than when the PI3K inhibitor is administered alone. In some embodiments, the frequency of administration of the second agent that achieves a therapeutic effect is lower (e.g., at least 20%, 30%, 40%, or 50% lower), when the second agent is administered in combination with PI3K inhibitor than when the second agent is administered alone.

In another aspect, provided herein is a method of treating, managing, or preventing a cancer in a subject. The method comprises administering to the subject a PI3K inhibitor (e.g., one or more PI3K inhibitors), or a pharmaceutically acceptable form thereof, in combination with a second agent (e.g., one or more second therapeutic agents), or pharmaceutically acceptable form thereof. In certain embodiments, the second agent is chosen from one or more of: 1) a checkpoint modulator, 2) an XPO1 inhibitor, 3) an anti-CD19 antibody, 4) a TLR agonist, 5) a STING agonist, or 6) a Flt3 ligand, or a combination thereof. In another aspect, provided herein is a composition for use in the treatment of a cancer. The composition for use in the treatment of cancer comprises a PI3K inhibitor (e.g., one or more PI3K inhibitors), or a pharmaceutically acceptable form thereof, in combination with a second agent (e.g., one or more second therapeutic agents), or pharmaceutically acceptable form thereof. The PI3K inhibitor and the second therapeutic agent can be present in a single dose form, or as two or more dose forms.

The combination of the PI3K inhibitor and the second agent can be administered together in a single composition or administered separately in two or more different compositions, e.g., pharmaceutical compositions or dosage forms as described herein. The administration of the PI3K inhibitor and the second agent can be in any order. For example, the PI3K inhibitor can be administered concurrently with, prior to, or subsequent to, the second agent. In one embodiment, the second agent is administered to a subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the second agent is administered concurrently with the PI3K inhibitor (e.g., Compound 1), or a pharmaceutically acceptable form thereof, e.g., in a single dosage form or separate dosage forms. In yet another embodiment, the second agent is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered. In some embodiments, the PI3K inhibitor and the second agent are administered with a timing that results in both agents being present at therapeutic levels at the same time in the patient. In some embodiments, the PI3K inhibitor and the second agent are administered sequentially. In some embodiments, administration of the PI3K inhibitor and the second agent overlaps in part with each other. In some embodiments, initiation of administration of the PI3K inhibitor and the second agent occurs at the same time. In some embodiments, the PI3K inhibitor is administered before initiating treatment with the second agent. In some embodiments, the second agent is administered before initiating treatment with the PI3K inhibitor. In some embodiments, the administration of the PI3K inhibitor continues after cessation of the administration of the second agent. In some embodiments, the administration of the second agent continues after cessation of the administration of the PI3K inhibitor.

In some embodiments, the combination of the PI3K inhibitor and the second agent is additive, e.g., the effect of the combination is similar to their individual effects added together. In certain embodiments, the combination of the PI3K inhibitor and the second agent is synergistic, e.g., has a synergistic effect in treating the cancer (e.g., in reducing cancer cell growth or viability, or both). In some embodiments, the amount or dosage of the PI3K inhibitor, the second agent, or both, used in combination does not exceed the level at which each agent is used individually, e.g., as a monotherapy. In certain embodiments, the amount or dosage of the PI3K inhibitor, the second agent, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the second agent, or both, used in combination that results in treatment of cancer is lower (e.g., at least 20%, at least 30%, at least 40%, or at least 50% lower) than the amount or dosage of each agent used individually, e.g., as a monotherapy. In certain embodiments, the frequency of administration of the PI3K inhibitor, the second agent, or both, used in combination that results in treatment of cancer is lower (e.g., at least 20%, 30%, 40%, or 50% lower), than the frequency of administration of each agent used individually, e.g., as a monotherapy.

The combination of PI3K inhibitor and the second agent can be administered during periods of active disorder, or during a period of remission or less active disease. The combination can be administered before a third treatment (e.g., a third therapeutic agent or a procedure (e.g., radiation or surgery)), concurrently with the third treatment, after the third treatment, or during remission of the disorder.

In another aspect, provided herein is a method of inhibiting the growth, the viability, or both, of a cancer cell, comprising contacting the cancer cell with a PI3K inhibitor (e.g., one or more PI3K inhibitors), or a pharmaceutically acceptable form thereof, in combination with a second agent (e.g., one or more second therapeutic agents), or pharmaceutically acceptable form thereof. In certain embodiments, the second agent is chosen from one or more of: 1) a checkpoint modulator, 2) an XPO1 inhibitor, 3) an anti-CD19 antibody, 4) a TLR agonist, 5) a STING agonist, or 6) a Flt3 ligand, or a combination thereof. The methods described herein can be used in vitro or in vivo, e.g., in an animal subject or as part of a therapeutic protocol.

The contacting of the cell with the PI3K inhibitor and the second agent can be in any order. In certain embodiments, the cell is contacted with the PI3K inhibitor concurrently, prior to, or subsequent to, the second agent. In certain embodiments, the combination of the PI3K inhibitor and the second agent is synergistic, e.g., has a synergistic effect in reducing cancer cell growth or viability, or both. In some embodiments, the amount or dosage of the PI3K inhibitor, the second agent, or both, used in combination does not exceed the level at which each agent is used individually, e.g., as a monotherapy. In certain embodiments, the amount or dosage of the PI3K inhibitor, the second agent, or both, used in combination is lower (e.g., at least 20%, at least 30%, at least 40%, or at least 50% lower) than the amount or dosage of each agent used individually, e.g., as a monotherapy.

In another aspect, provided herein is a synergistic combination of a PI3K inhibitor, or a pharmaceutically acceptable form thereof, and a second therapeutic agent, or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand, or a combination thereof, for use in treating cancer. In another aspect, provided herein is a synergistic combination of a PI3K inhibitor, or a pharmaceutically acceptable form thereof, and a second therapeutic agent, or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand, or a combination thereof, for use in a medicament. In another aspect, provided herein is a use of a synergistic combination of a PI3K inhibitor, or a pharmaceutically acceptable form thereof, and a second therapeutic agent, or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand, or a combination thereof, for treating cancer. In another aspect, provided herein is a use of a synergistic combination of a PI3K inhibitor, or a pharmaceutically acceptable form thereof, and a second therapeutic agent, or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand, or a combination thereof for the manufacture of a medicament for treating cancer.

Additional features or embodiments of the compositions or methods described herein include one or more of the following:

In certain embodiments, the combination of the PI3K inhibitor and the second agent used in the compositions and methods described herein is synergistic, e.g., as indicated by a combination index value that is less than 1 for the combination of the PI3K inhibitor and the second agent. In certain embodiments, the combination is synergistic as indicated by a combination index value that is less than 0.7 for the combination of the PI3K inhibitor and the second agent. In certain embodiments, the combination is synergistic as indicated by a combination index value that is less than 0.5 for the combination of the PI3K inhibitor and the second agent. In certain embodiments, the combination is synergistic as indicated by a combination index value that is less than 0.7, 0.6, 0.5, 0.4, 0.3, 0.2, or 0.1 for the combination of the PI3K inhibitor and the second agent. In some embodiments, the combination of the PI3K inhibitor and the second agent used in the compositions and methods described herein is additive, e.g., as indicated by a combination index value that is equal to about 1 for the combination of the PI3K inhibitor and the second agent. In certain embodiments, the combination index value is assessed at 50% inhibition, e.g., as described herein in the Examples. In certain embodiments, the combination index value is assessed at 50% growth inhibition, e.g., as described herein in the Examples. In certain embodiments, the combination index value is assessed at 10%, 20%, 30%, 40%, 50%, 60%, 60%, 70%, 80%, or 90% inhibition or growth inhibition. In certain embodiments, the combination index value is calculated as described herein in the Examples.

In other embodiments, the combination of the PI3K inhibitor and the second agent used in the compositions and methods described herein is synergistic, e.g., as indicated by a synergy score value of greater than 1, 2, or 3. In certain embodiments, the combination is synergistic as indicated by a synergy score value of greater than 1. In certain embodiments, the combination is synergistic as indicated by a synergy score value of greater than 2. In certain embodiments, the combination is synergistic as indicated by a synergy score value of greater than 3. In some embodiments, the combination of the PI3K inhibitor and the second agent used in the compositions and methods described herein is additive, e.g., as indicated by a synergy score value of zero. In certain embodiments, the synergy score is calculated as described herein in the Examples.

In some embodiments, the anti-cancer effect provided by the combination of the PI3K inhibitor and the second agent used in the compositions and methods described herein is greater than the anti-cancer effect provided by an agent (e.g., the PI3K inhibitor or the second agent) used individually, e.g., as a monotherapy. In certain embodiments, the anti-cancer effect provided by the combination of the PI3K inhibitor and the second agent is at least 2 fold greater, at least 3 fold greater, at least 5 fold greater, or at least 10 fold greater than the anti-cancer effect provided by an agent used individually, e.g., as a monotherapy (e.g., by a monotherapy with the same dose of the PI3K inhibitor, or by a monotherapy with the same dose of the second agent).

In some embodiments, the anti-cancer effect provided by the combination of the PI3K inhibitor and the second agent used in the compositions and methods described herein is greater than the anti-cancer effect provided by a monotherapy with the same dose of the PI3K inhibitor. In certain embodiments, the anti-cancer effect provided by the combination is at least 2 fold greater, at least 3 fold greater, at least 5 fold greater, or at least 10 fold greater than the anti-cancer effect provided by the monotherapy with the same dose of the PI3K inhibitor.

In some embodiments, the anti-cancer effect of the combination of the PI3K inhibitor and the second agent used in the compositions and methods described herein is greater than the anti-cancer effect provided by a monotherapy with the same dose of the second agent. In certain embodiments, the anti-cancer effect of the combination of the PI3K inhibitor and the second agent is at least 2 fold greater, at least 3 fold greater, at least 5 fold greater, or at least 10 fold greater than the anti-cancer effect provided by the monotherapy with the same dose of the second agent.

In some embodiments, one or more side effects of the PI3K inhibitor, the second agent, or both, is reduced compared with the side effects of each agent when used individually, e.g., as a monotherapy (e.g., a monotherapy comprising the PI3K inhibitor without the second agent at a dose that achieves the same therapeutic effect; or a monotherapy comprising the second agent without the PI3K inhibitor). For example, a reduction, prevention, delay, or decrease in the occurrence or the likelihood of occurrence of one or more side effects, toxicity, or resistance, that would otherwise be associated with administration of at least one of the agents, e.g., the PI3K inhibitor.

In some embodiments, one or more side effects of the compositions or methods described herein is reduced compared with the side effects of a monotherapy comprising either the second agent (or pharmaceutically acceptable form thereof) or the PI3K inhibitor (or pharmaceutically acceptable form thereof) at a dose that achieves the same therapeutic effect.

In some embodiments, said one or more side effects includes a liver enzyme level, e.g., a liver enzyme level indicative of toxicity.

In some embodiments, the combination of the PI3K inhibitor and the second agent used in the compositions and methods described herein results in a reduction in resistance (e.g., a decrease in a measure of resistance or a decreased likelihood of developing resistance), or a delay in the development of resistance, to at least one of the agents, e.g., resistance (e.g., acquired resistance) to the PI3K inhibitor.

In some embodiments, the combination of the PI3K inhibitor and the second agent used in the compositions and methods described herein results in a reduction in minimal residual disease (MRD). In certain embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination was administered). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent. In certain embodiments, the MRD is decreased below the level observed during treatment with a monotherapy comprising the PI3K inhibitor. In certain embodiments, the MRD is decreased below the level observed during treatment with a monotherapy comprising the second agent. In certain embodiments, the combination is effective to reduce the level of MRD below a preselected cutoff value (e.g., 1 malignant cell in 100 normal cells, 1 malignant cell in 1000 normal cells, or 1 malignant cell in 10,000 normal cells). In certain embodiments, the preselected cutoff value is 1 malignant cell in 1000 or 10,000 normal cells. In some embodiments, a subject exhibits MRD negativity (or is MRD-negative) if the MRD is below a preselected cutoff value (e.g., a preselected cutoff value as described herein). In some embodiments, the level of MRD is not detectable by standard laboratory methodologies.

In another aspect, provided herein is a method of decreasing the level of MRD in a subject having a cancer. The method comprises:
(a) administering to the subject a PI3K inhibitor (e.g., Compound 1), or a pharmaceutically acceptable form thereof, in combination with a second agent (e.g., a second agent chosen from one or more of a checkpoint modulator, an XPO1 inhibitor, an anti-CD19 antibody, a TLR agonist, a STING agonist, or a Flt3 ligand, or a combination thereof, as described herein) (also referred to as “a first treatment”);
(b) monitoring the level of MRD in the subject, e.g., by one or more methods described herein or known in the art (e.g., flow cytometry, sequencing, or PCR); and
(c) if the subject has a level of MRD below a preselected cutoff value ((e.g., 1 malignant cell in 100 normal cells, 1 malignant cell in 1000 normal cells, or 1 malignant cell in 10,000 normal cells), e.g., for a time period after therapy (e.g., at least 1, 2, 3, 6, 9, 12 months)), alter the combination treatment (e.g., reduce the dose or frequency (e.g., by about 20%, 30%, 40%, or 50%) of the PI3K inhibitor, the second agent, or both, or cease the first treatment).

In some embodiments, the method further comprises monitoring the subject after altering the combination treatment (e.g., after reducing the dose or frequency (e.g., by about 20%, 30%, 40%, or 50%) of the PI3K inhibitor, the second agent, or both, or ceasing the first treatment), (e.g., for a period of at least 6 months, 9 months or 12 months), and if the level of MRD increases, e.g., increases above a preselected cutoff value (e.g., a preselected cutoff value as described herein (e.g., 1 malignant cell in 100 normal cells, 1 malignant cell in 1000 normal cells, or 1 malignant cell in 10,000 normal cells)), a second treatment is administered. In one embodiment, the second treatment is a PI3K inhibitor monotherapy. In another embodiment, the second treatment comprises a PI3K inhibitor in combination with a second agent (e.g., a second agent as described herein, e.g., one or more of a checkpoint modulator, an XPO1 inhibitor, an anti-CD19 antibody, a TLR agonist, a STING agonist, or a Flt3 ligand, or a combination thereof, as described herein). In one embodiment, the second treatment includes the same second agent as the first treatment. In another embodiment, the second treatment includes a different second agent as the first treatment. In yet another embodiment, the second treatment comprises a PI3K inhibitor in combination with a third agent (e.g., an anti-CD20 antibody or a BTK inhibitor such as ibrutinib). In yet another embodiment, the second treatment comprises a PI3K inhibitor, a second agent (e.g., a second agent as described herein, e.g., one or more of a checkpoint modulator, an XPO1 inhibitor, an anti-CD19 antibody, a TLR agonist, a STING agonist, or a Flt3 ligand, or a combination thereof, as described herein) and a third agent (e.g., an anti-CD20 antibody or a BTK inhibitor such as ibrutinib).

In another aspect, provided herein is a method of decreasing the level of MRD detected in a subject having a cancer. The method comprises:
(a) administering to the subject a PI3K inhibitor (e.g., Compound 1), or a pharmaceutically acceptable form thereof, in combination with a second agent (e.g., a second agent chosen from one or more of a checkpoint modulator, an XPO1 inhibitor, an anti-CD19 antibody, a TLR agonist, a STING agonist, or a Flt3 ligand, or a combination thereof, as described herein) (also referred to as “a first treatment”);
(b) monitoring the level of MRD in the subject, e.g., by one or more methods described herein or known in the art (e.g., flow cytometry, sequencing, or PCR); and
(c) stop administering the first treatment (e.g., the combination) if the level of MRD in the subject decreases below a preselected cutoff value (e.g., 1 malignant cell in 100 normal cells, 1 malignant cell in 1000 normal cells, or 1 malignant cell in 10,000 normal cells).

In some embodiments, the method further comprises (d) monitoring the level of MRD in the subject, e.g., by one or more of the methods described herein or known in the art (e.g., flow cytometry, sequencing, or PCR) and (e) administering a second treatment (e.g., a monotherapy comprising a PI3K inhibitor, or administering a further combination comprising the PI3K inhibitor, or a pharmaceutically acceptable form thereof), if the level of MRD increases, e.g., increase above a preselected cutoff value (e.g., 1 malignant cell in 100 normal cells, 1 malignant cell in 1000 normal cells, or 1 malignant cell in 10,000 normal cells). In one embodiment, steps (b), (c), (d) and (e) are repeated one or more times. In one embodiment the second treatment is a PI3K inhibitor monotherapy. In another embodiment, the second treatment comprises a PI3K inhibitor in combination with a second agent (e.g., a second agent as described herein, e.g., one or more of a checkpoint modulator, an XPO1 inhibitor, an anti-CD19 antibody, a TLR agonist, a STING agonist, or a Flt3 ligand, or a combination thereof, as described herein). In one embodiment, the second treatment includes the same second agent as the first treatment. In another embodiment, the second treatment includes a different second agent as the first treatment. In yet another embodiment, the second treatment comprises a PI3K inhibitor in combination with a third agent (e.g., an anti-CD20 antibody or a BTK inhibitor such as ibrutinib). In yet another embodiment, the second treatment comprises a PI3K inhibitor, a second agent (e.g., a second agent as described herein, e.g., one or more of a checkpoint modulator, an XPO1 inhibitor, an anti-CD19 antibody, a TLR agonist, a STING agonist, or a Flt3 ligand, or a combination thereof, as described herein) and a third agent (e.g., an anti-CD20 antibody or a BTK inhibitor such as ibrutinib).

The aforesaid compositions and methods can be used in combination with a monotherapy (e.g., a monotherapeutic administration or dose of the PI3K inhibitor, the second agent or a third agent). In one embodiment, the subject is administered a monotherapy with a PI3K inhibitor, which can be followed with a combination composition or method described herein. For example, if the subject is developing, or is identified as developing, a decreased responsiveness to a first monotherapy, (e.g., with a PI3K inhibitor, a second agent, or third agent), any of the combination compositions or methods described herein can be administered. In certain embodiments, the combination compositions or methods described herein improve responsiveness (e.g., as indicated by a decrease in the level of MRD, e.g., a decrease below the level of MRD observed during treatment with the first monotherapy). Alternatively, administration of any of the combination compositions or methods described herein can be followed by administration of a monotherapy, e.g., with a PI3K inhibitor, the second agent, or third agent.

In other embodiments, the composition and methods described herein can include further agents or therapies, including but not limited to, chemotherapeutics, radiation, or surgery.

In some embodiments, the PI3K inhibitor is chosen from one or more of Compound 1, AMG-319, GSK 2126458, GSK 1059615, GDC-0032, GDC-0980, GDC-0941, XL147, XL499, XL765, BKM 120, GS1101, CAL 263, SF1126, PX-866, BEZ235, CAL-120, BYL719, RP6503, RP6530, TGR1202, INK1117, PX-886, BAY 80-6946, IC87114, Palomid 529, ZSTK474, PWT33597, TG100-115, GNE-477, CUDC-907, AEZS-136, BGT-226, PF-05212384, LY3023414, PI-103, LY294002, INCB-040093, CAL-130 and wortmannin.

In one embodiment, the PI3K inhibitor is Compound 1, or a pharmaceutically acceptable form thereof. Compound 1 has the chemical name of (S)-3-(1-((9H-purin-6-yl)amino)ethyl)-8-chloro-2-phenylisoquinolin-1(2H)-one, and is of the following structure:

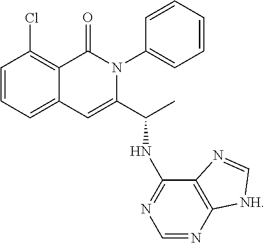 [SMILES: C[C@H](Nc1ncnc2[nH]cnc12)c1cc2cccc(Cl)c2c(=O)n1-c1ccccc1]

In one embodiment, the PI3K inhibitor is Idelalisib (GS1101, CAL-101), or a pharmaceutically acceptable form thereof. Idelalisib (GS1101, CAL-101) has the chemical name of (S)-2-(1-(9H-purin-6-ylamino)propyl)-5-fluoro-3-phenylquinazolin-4(3H)-one, and is of the following structure:

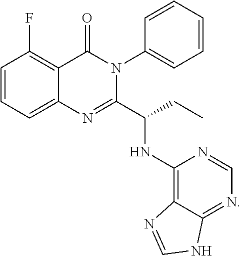 [SMILES: CC[C@H](Nc1ncnc2[nH]cnc12)c1nc2cccc(F)c2c(=O)n1-c1ccccc1]

In certain embodiments of the compositions and methods described herein, the PI3K inhibitor is a PI3K delta inhibitor. In one embodiment, the PI3K inhibitor is a dual inhibitor of PI3K delta/gamma.

The combinations described herein can further comprise a third therapeutic agent which is a chemotherapeutic agent. The chemotherapeutic agent can be, for example, bendamustine, chlorambucil, cyclophosphamide, doxorubicin, vincristine, fludarabine, or any combination thereof such as CHOP (cyclophosphamide, doxorubicin, vincristine, prednisone) or FC (fludarabine, cyclophosphamide).

In some embodiments, the pharmaceutical composition further comprises a pharmaceutically acceptable excipient (e.g., one or more pharmaceutically acceptable excipients).

In some embodiments of the compositions and methods described herein, the combination of the PI3K inhibitor and the second agent is therapeutically effective (e.g., synergistically effective), in treating a cancer in the subject, e.g., for treatment of a cancer described herein.

In one embodiment, the cancer is of hematopoietic origin. In one embodiment, the cancer is lymphoma or leukemia. In one embodiment, the cancer is B-cell lymphoma, mantle cell lymphoma, non-Hodgkin's lymphoma (e.g., non-Hodgkin's B-cell lymphoma), T-cell lymphoma, cutaneous lymphoma, anaplastic large cell lymphoma, multiple myeloma, myeloma, or plasmacytoma. In one embodiment, the cancer is a multiple myeloma. In one embodiment, the cancer is a chronic lymphocytic leukemia (CLL).

In other embodiments, the cancer is a non-Hodgkin's lymphoma. In certain embodiments, the cancer is a B cell non-Hodgkin's lymphoma. In certain embodiments, the non-Hodgkin's lymphoma is a diffuse large B-cell lymphoma. In certain embodiments, the non-Hodgkin's lymphoma is a diffuse large B-cell lymphoma activated B-cell like or a diffuse large B-cell lymphoma germinal center B-cell-like. In certain embodiments, the cancer is an indolent non-Hodgkin's lymphoma, e.g., a follicular lymphoma. In certain embodiments, the cancer is a mantle cell lymphoma. In certain embodiments, the cancer is a T-cell non-Hodgkin's lymphoma.

In some embodiments, the cancer is a T cell lymphoma, e.g., a peripheral T cell lymphoma (PTCL) or a cutaneous T cell lymphoma (CTCL).

In one embodiment, the subject is a mammal, e.g., a human. In one embodiment, the subject is at risk or suffers from a cancer, e.g., a cancer described herein.

In one embodiment, the method delays resistance of the cancer, e.g., to a therapeutic agent, e.g., to the PI3K inhibitor such as Compound 1, or to the second agent. In one embodiment, the method reduces the risk that the cancer becomes resistant, e.g., to a therapeutic agent, e.g., to the PI3K inhibitor such as Compound 1, or to the second agent. In one embodiment, the cancer does not become resistant (e.g., to the PI3K inhibitor) for at least 1, 2, 3, 4, 5, 6, 7, 8, 9, 10, 11, 12, 18, 24, 30, or 36 months. In one embodiment, the method prolongs remission (e.g., complete remission or partial remission) in the subject. In one embodiment, the subject experiences remission (e.g., complete remission or partial remission) for at least 1, 2, 3, 4, 5, 6, 7, 8, 9, 10, 11, 12, 18, 24, 30, or 36 months. In one embodiment, the method increases the likelihood that the subject experiences complete remission. In one embodiment, the subject experiences complete remission. In one embodiment, the method results in a reduction in the level of minimal residual disease (MRD). In one embodiment, the subject has substantially no detectable MRD. In certain embodiments, the subject displays one or more of these characteristics (e.g., remission) after treatment with the PI3K inhibitor and the second agent for a therapeutically effective period of time, e.g., at least 1, 2, 3, or 4 weeks, or 1, 2, 4, 6, 9, or 12 months.

In one embodiment, the subject shows decreased responsiveness to a PI3K inhibitor (e.g., is resistant or refractive to treatment with a PI3K inhibitor, e.g., Compound 1). In one embodiment, the subject is identified as having a decreased susceptibility (e.g., resistance or acquired resistance) to a monotherapy treatment with a PI3K inhibitor (e.g., Compound 1 or Idelalisib), or a pharmaceutically acceptable form thereof. In one embodiment, the subject is identified as having a decreased susceptibility (e.g., resistance or acquired resistance) to a monotherapy treatment of a PI3K inhibitor (e.g., Compound 1), or a pharmaceutically acceptable form thereof. In one embodiment, the subject is identified as having an increased susceptibility to a combination therapy treatment provided herein.

In some embodiments of the compositions and methods described herein, the PI3K inhibitor and the second therapeutic agent are the only therapeutically active ingredients for treating a cancer.

Additional combinations of three or more agents are encompassed by the methods and compositions described herein.

In certain embodiments, provided herein is a composition (e.g., a pharmaceutical composition) comprising a PI3K inhibitor, e.g., one or more PI3K inhibitors (e.g., Compound 1 or Idelalisib, or both), or a pharmaceutically acceptable form thereof, in combination with a checkpoint modulator (e.g., one or more checkpoint modulators), or a pharmaceutically acceptable form thereof. The PI3K inhibitor and the checkpoint modulator can be present in a single composition or as two or more different compositions. In some embodiments, the composition (e.g., one or more compositions comprising the combination of PI3K inhibitor and the checkpoint modulator) is synergistic, e.g., has a synergistic effect in treating a cancer (e.g., in reducing cancer cell growth or viability, or both, e.g., as described herein). In certain embodiments, the amount or dosage of the PI3K inhibitor, the checkpoint modulator, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject comprising administering to the subject a PI3K inhibitor, e.g., one or more PI3K inhibitors (e.g., Compound 1 or Idelalisib, or both) or a pharmaceutically acceptable form thereof, in combination with a checkpoint modulator (e.g., one or more checkpoint modulators), or a pharmaceutically acceptable form thereof. In certain embodiments, the combination of the PI3K inhibitor and the checkpoint modulator is synergistic, e.g., has a synergistic effect in treating the cancer (e.g., in reducing cancer cell growth or viability, or both). In some embodiments, the amount or dosage of the PI3K inhibitor, the checkpoint modulator, or both, used in combination does not exceed the level at which each agent is used individually, e.g., as a monotherapy. In certain embodiments, the amount or dosage of the PI3K inhibitor, the checkpoint modulator, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the checkpoint modulator, or both, used in combination that results in treatment of cancer is lower (e.g., at least 20%, at least 30%, at least 40%, or at least 50% lower) than the amount or dosage of each agent used individually, e.g., as a monotherapy.

In one embodiment of the compositions and methods provided herein, the checkpoint modulator is an anti-PD-1 antibody, an anti-PD-L1 antibody, an anti-PD-L2 antibody, or an anti-CTLA-4 antibody, or a combination thereof. In one embodiment, the anti-PD-1 antibody is Nivolumab, Pembrolizumab, Pidilizumab, AMP-514, or AMP-224, or a combination thereof. In one embodiment, the anti-PD-L1 antibody is MDX-1105, YW243.55.S70, MDPL3280A, MSB0010718C, or durvalumab, or a combination thereof. In one embodiment, the an anti-CTLA-4 antibody is Tremelimumab or Ipilimumab, or a combination thereof.

In certain embodiments, provided herein is a composition (e.g., one or more pharmaceutical compositions or dosage forms), comprising a PI3K inhibitor, e.g., one or more PI3K inhibitors (e.g., Compound 1 or Idelalisib, or both) or a pharmaceutically acceptable form thereof, in combination with an XPO1 inhibitor (e.g., one or more XPO1 inhibitors), or a pharmaceutically acceptable form thereof. The PI3K inhibitor and the XPO1 inhibitor can be present in a single composition or as two or more different compositions. In some embodiments, the composition (e.g., one or more compositions comprising the combination of PI3K inhibitor and the XPO1 inhibitor) is synergistic, e.g., has a synergistic effect in treating a cancer (e.g., in reducing cancer cell growth or viability, or both, e.g., as described herein). In certain embodiments, the amount or dosage of the PI3K inhibitor, the XPO1 inhibitor, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject comprising administering to the subject a PI3K inhibitor, e.g., one or more PI3K inhibitors (e.g., Compound 1 or Idelalisib, or both) or a pharmaceutically acceptable form thereof, in combination with an XPO1 inhibitor (e.g., one or more XPO1 inhibitors), or a pharmaceutically acceptable form thereof. In certain embodiments, the combination of the PI3K inhibitor and the XPO1 inhibitor is synergistic, e.g., has a synergistic effect in treating the cancer (e.g., in reducing cancer cell growth or viability, or both). In some embodiments, the amount or dosage of the PI3K inhibitor, the XPO1 inhibitor, or both, used in combination does not exceed the level at which each agent is used individually, e.g., as a monotherapy. In certain embodiments, the amount or dosage of the PI3K inhibitor, the XPO1 inhibitor, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the XPO1 inhibitor, or both, used in combination that results in treatment of cancer is lower (e.g., at least 20%, at least 30%, at least 40%, or at least 50% lower) than the amount or dosage of each agent used individually, e.g., as a monotherapy.

In one embodiment of the methods and compositions described herein, the XPO1 inhibitor is selinexor, KPT-251, KPT-276, or SL-801, or a combination thereof. In one embodiment, the XPO1 inhibitor is selinexor.

In certain embodiments, provided herein is a composition (e.g., a pharmaceutical composition) comprising a PI3K inhibitor (e.g., Compound 1 or Idelalisib), or a pharmaceutically acceptable form thereof, in combination with an anti-CD19 antibody (e.g., one or more anti-CD19 antibodies), or a pharmaceutically acceptable form thereof. The PI3K inhibitor and the anti-CD19 antibody can be present in a single composition or as two or more different compositions. In some embodiments, the composition (e.g., one or more compositions comprising the combination of PI3K inhibitor and the anti-CD19 antibody) is synergistic, e.g., has a synergistic effect in treating a cancer (e.g., in reducing cancer cell growth or viability, or both, e.g., as described herein). In certain embodiments, the amount or dosage of the PI3K inhibitor, the anti-CD19 antibody, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject comprising administering to the subject a PI3K inhibitor, e.g., one or more PI3K inhibitors (e.g., Compound 1 or Idelalisib, or both) or a pharmaceutically acceptable form thereof, in combination with an anti-CD19 antibody (e.g., one or more anti-CD19 antibodies), or a pharmaceutically acceptable form thereof. In certain embodiments, the combination of the PI3K inhibitor and the anti-CD19 antibody is synergistic, e.g., has a synergistic effect in treating the cancer (e.g., in reducing cancer cell growth or viability, or both). In some embodiments, the amount or dosage of the PI3K inhibitor, the anti-CD19 antibody, or both, used in combination does not exceed the level at which each agent is used individually, e.g., as a monotherapy. In certain embodiments, the amount or dosage of the PI3K inhibitor, the anti-CD19 antibody, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the anti-CD19 antibody, or both, used in combination that results in treatment of cancer is lower (e.g., at least 20%, at least 30%, at least 40%, or at least 50% lower) than the amount or dosage of each agent used individually, e.g., as a monotherapy.

In one embodiment of the methods and compositions described herein, the anti-CD19 antibody is blinatumomab.

In certain embodiments, provided herein is a composition, e.g., one or more pharmaceutical composition, comprising a PI3K inhibitor, e.g., one or more PI3K inhibitors (e.g., Compound 1 or Idelalisib), or a pharmaceutically acceptable form thereof, in combination with a TLR agonist (e.g., one or more TLR agonists), or a pharmaceutically acceptable form thereof. The PI3K inhibitor and the TLR agonist can be present in a single composition or as two or more different compositions. In some embodiments, the composition (e.g., one or more compositions comprising the combination of PI3K inhibitor and the TLR agonist) is synergistic, e.g., has a synergistic effect in treating a cancer (e.g., in reducing cancer cell growth or viability, or both, e.g., as described herein). In certain embodiments, the amount or dosage of the PI3K inhibitor, the TLR agonist, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject. The method includes administering to the subject a PI3K inhibitor, e.g., one or more PI3K inhibitors (e.g., Compound 1 or Idelalisib, or both) or a pharmaceutically acceptable form thereof, in combination with a TLR agonist (e.g., one or more TLR agonists), or a pharmaceutically acceptable form thereof. In certain embodiments, the combination of the PI3K inhibitor and the TLR agonist is synergistic, e.g., has a synergistic effect in treating the cancer (e.g., in reducing cancer cell growth or viability, or both). In some embodiments, the amount or dosage of the PI3K inhibitor, the TLR agonist, or both, used in combination does not exceed the level at which each agent is used individually, e.g., as a monotherapy. In certain embodiments, the amount or dosage of the PI3K inhibitor, the TLR agonist, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the TLR agonist, or both, used in combination that results in treatment of cancer is lower (e.g., at least 20%, at least 30%, at least 40%, or at least 50% lower) than the amount or dosage of each agent used individually, e.g., as a monotherapy.

In certain embodiments, provided herein is a composition, e.g., one or more pharmaceutical composition, comprising a PI3K inhibitor, e.g., one or more PI3K inhibitors (e.g., Compound 1 or Idelalisib), or a pharmaceutically acceptable form thereof, in combination with a STING agonist (e.g., one or more STING agonists), or a pharmaceutically acceptable form thereof. The PI3K inhibitor and the STING agonist can be present in a single composition or as two or more different compositions. In some embodiments, the composition (e.g., one or more compositions comprising the combination of PI3K inhibitor and the STING agonist) is synergistic, e.g., has a synergistic effect in treating a cancer (e.g., in reducing cancer cell growth or viability, or both, e.g., as described herein). In certain embodiments, the amount or dosage of the PI3K inhibitor, the STING agonist, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject. The method includes administering to the subject a PI3K inhibitor, e.g., one or more PI3K inhibitors (e.g., Compound 1 or Idelalisib, or both) or a pharmaceutically acceptable form thereof, in combination with a STING agonist (e.g., one or more STING agonists), or a pharmaceutically acceptable form thereof. In certain embodiments, the combination of the PI3K inhibitor and the STING agonist is synergistic, e.g., has a synergistic effect in treating the cancer (e.g., in reducing cancer cell growth or viability, or both). In some embodiments, the amount or dosage of the PI3K inhibitor, the STING agonist, or both, used in combination does not exceed the level at which each agent is used individually, e.g., as a monotherapy. In certain embodiments, the amount or dosage of the PI3K inhibitor, the STING agonist, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the STING agonist, or both, used in combination that results in treatment of cancer is lower (e.g., at least 20%, at least 30%, at least 40%, or at least 50% lower) than the amount or dosage of each agent used individually, e.g., as a monotherapy.

In certain embodiments, provided herein is a composition, e.g., one or more pharmaceutical composition, comprising a PI3K inhibitor, e.g., one or more PI3K inhibitors (e.g., Compound 1 or Idelalisib), or a pharmaceutically acceptable form thereof, in combination with a Flt3 ligand (e.g., one or more Flt3 ligands), or a pharmaceutically acceptable form thereof. The PI3K inhibitor and the Flt3 ligand can be present in a single composition or as two or more different compositions. In some embodiments, the composition (e.g., one or more compositions comprising the combination of PI3K inhibitor and the Flt3 ligand) is synergistic, e.g., has a synergistic effect in treating a cancer (e.g., in reducing cancer cell growth or viability, or both, e.g., as described herein). In certain embodiments, the amount or dosage of the PI3K inhibitor, the Flt3 ligand, or both, present in the composition(s) is lower (e.g., at least 20%, at least 30%, at least 40%, or at least 50% lower) than the amount or dosage of each agent used individually, e.g., as a monotherapy.

In certain embodiments, provided herein is a method of treating, managing, or preventing a cancer in a subject. The method includes administering to the subject a PI3K inhibitor, e.g., one or more PI3K inhibitors (e.g., Compound 1 or Idelalisib, or both) or a pharmaceutically acceptable form thereof, in combination with a Flt3 ligand (e.g., one or more Flt3 ligands), or a pharmaceutically acceptable form thereof. In certain embodiments, the combination of the PI3K inhibitor and the Flt3 ligand is synergistic, e.g., has a synergistic effect in treating the cancer (e.g., in reducing cancer cell growth or viability, or both). In some embodiments, the amount or dosage of the PI3K inhibitor, the Flt3 ligand, or both, used in combination does not exceed the level at which each agent is used individually, e.g., as a monotherapy. In certain embodiments, the amount or dosage of the PI3K inhibitor, the Flt3 ligand, or both, used in combination is lower (e.g., at least 20%, at least 30%, at least 40%, or at least 50% lower) than the amount or dosage of each agent used individually, e.g., as a monotherapy. In other embodiments, the amount or dosage of the PI3K inhibitor, the Flt3 ligand, or both, used in combination that results in treatment of cancer is lower (e.g., at least 20%, at least 30%, at least 40%, or at least 50% lower) than the amount or dosage of each agent used individually, e.g., as a monotherapy.

Embodiments relating to dosages of the agents included in the compositions and methods described herein follow. In one embodiment, the PI3K inhibitor, e.g., Compound 1, is administered at a dosage of from about 0.01 mg to about 75 mg daily, and the second therapeutic agent is administered at a dosage of from about 0.01 to about 1100 mg daily.

In certain embodiments, the amount or dosage of the PI3K inhibitor, the second agent, or both, that is used in the method or composition is lower (e.g., at least 20%, at least 30%, at least 40%, at least 50%, at least 60%, at least 70%, or at least 80% lower) than the amount or dosage of each agent used individually, e.g., as a monotherapy. In other embodiments, the amount or dosage of the PI3K inhibitor, the second agent, or both, present in the composition(s) that results in a desired effect (e.g., treatment of cancer) is lower (e.g., at least 20%, at least 30%, at least 40%, at least 50%, at least 60%, at least 70%, or at least 80% lower) than the amount or dosage of each agent used individually, e.g., as a monotherapy.

In one embodiment, the molar ratio of the PI3K inhibitor, or the pharmaceutically acceptable form thereof, to the second therapeutic agent, or the pharmaceutically acceptable form thereof, is in the range of from about 10000:1 to about 1:10000.

In one embodiment, the composition comprises the PI3K inhibitor, or a pharmaceutically acceptable form thereof, at an amount of in the range of from about 0.01 mg to about 75 mg and the second therapeutic agent, or a pharmaceutically acceptable form thereof, at an amount of in the range of from about 0.01 mg to about 1100 mg.

In certain embodiments, the PI3K inhibitor is Compound 1 at a dosage of 25 mg (e.g., 25 mg BID). In certain embodiments, Compound 1 is effective as a monotherapy at a dosage of 25 mg (e.g., 25 mg BID). In certain embodiments, the combination of Compound 1 and the second agent is effective, e.g., in treating a cancer and/or in reducing cancer cell growth or viability, with Compound 1 at a dosage lower than 25 mg (e.g., 25 mg BID). In other embodiments, the dosage of Compound 1 included in the combination is 5 mg to 20 mg (e.g., 5 mg to 20 mg BID). In other embodiments, the dosage of Compound 1 included in the combination is 10 mg to 25 mg (e.g., 10 mg to 25 mg BID), 15 mg to 25 mg (e.g., 15 mg to 25 mg BID), 5 mg to 50 mg (e.g., 5 mg to 50 mg BID), 5 mg to 25 mg (e.g., 5 mg to 25 mg BID), 5 mg to 10 mg (e.g., 5 mg to 10 mg BID), 10 mg to 15 mg (e.g., 10 mg to 15 mg BID), 15 mg to 20 mg (e.g., 15 mg to 20 mg BID), 20 mg to 25 mg (e.g., 20 mg to 25 mg BID), 25 mg to 30 mg (e.g., 25 mg to 30 mg BID), 30 mg to 35 mg (e.g., 30 mg to 35 mg BID), 35 mg to 40 mg (e.g., 35 mg to 40 mg BID), 40 mg to 45 mg (e.g., 40 mg to 45 mg BID), or 45 mg to 50 mg (e.g., 45 mg to 50 mg BID). In certain embodiments, the dosage of Compound 1 is 22.5 mg (e.g., 22.5 mg BID), 20 mg (e.g., 20 mg BID), 17.5 mg (e.g., 17.5 mg BID), 15 mg (e.g., 15 mg BID), 12.5 mg (e.g., 12.5 mg BID), 10 mg (e.g., 10 mg BID), 7.5 mg (e.g., 7.5 mg BID), or 5 mg (e.g., 5 mg BID).

In some embodiments, the PI3K inhibitor, e.g., Compound 1, is administered at a dose frequency of twice per day (BID), once per day, once per two days, once per three days, once per four days, once per five days, once per six days, or once per week. In certain embodiments, the combination of the PI3K inhibitor (e.g., Compound 1) and the second agent is effective, e.g., in treating a cancer and/or in reducing cancer cell growth or viability, with the PI3K inhibitor (e.g., Compound 1) administered at a dose frequency of twice per day (BID), once per day, once per two days, once per three days, once per four days, once per five days, once per six days, or once per week.

In some embodiments, the PI3K inhibitor is Idelalisib at a dosage of 150 mg (e.g., 150 mg BID). In certain embodiments, Idelalisib is effective as a monotherapy at a dosage of 150 mg (e.g., 150 mg BID). In certain embodiments, the combination of Idelalisib and the second agent is effective, e.g., in treating a cancer and/or in reducing cancer cell growth or viability, with Idelalisib at a dosage lower than 150 mg (e.g., 150 mg BID). In some embodiments, the dosage of Idelalisib included in the combination is 30 mg to 135 mg (e.g., 30 mg to 135 mg BID). In certain embodiments, the dosage of Idelalisib is 135 mg (e.g., 135 mg BID), 120 mg (e.g., 120 mg BID), 105 mg (e.g., 105 mg BID), 90 mg (e.g., 90 mg BID), 75 mg (e.g., 75 mg BID), 60 mg (e.g., 60 mg BID), 45 mg (e.g., 45 mg BID), or 30 mg (e.g., 30 mg BID).

In some embodiments, the PI3K inhibitor is Idelalisib and is administered at a dose frequency of twice per day, once per day, once per two days, once per three days, once per four days, once per five days, once per six days, or once per week. In certain embodiments, the combination of Idelalisib and the second agent is effective, e.g., in treating a cancer and/or in reducing cancer cell growth or viability, with Idelalisib administered at a dose frequency of twice per day (BID), once per day, once per two days, once per three days, once per four days, once per five days, once per six days, or once per week.

In one embodiment, the second agent is administered to a subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the second agent is administered concurrently with the PI3K inhibitor (e.g., Compound 1), or a pharmaceutically acceptable form thereof, e.g., in a single dosage form or separate dosage forms. In yet another embodiment, the second agent is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In one embodiment, provided herein is a method of reducing the likelihood for a subject to develop resistance to a treatment with a PI3K inhibitor, comprising:
(a) administering to the subject a therapeutically effective amount of a monotherapy comprising the PI3K inhibitor, or a pharmaceutically acceptable form thereof, for a first period of time;
(b) after the first period of time, administering to the subject a therapeutically effective amount of a combination therapy comprising the PI3K inhibitor in combination with a second agent or a pharmaceutically acceptable form thereof, wherein the second agent is chosen from one or more of 1) a checkpoint modulator, 2) an XPO1 inhibitor, 3) an anti-CD19 antibody, 4) a TLR agonist, 5) a STING agonist, or 6) a Flt3 ligand, for a second period of time; and
(c) optionally repeating steps (a) and (b) one or more times.

In one embodiment, provided herein is a method of reducing the likelihood for a subject to develop resistance to a treatment with a PI3K inhibitor, comprising:
(a) administering to the subject a therapeutically effective amount of a monotherapy comprising the second agent, or a pharmaceutically acceptable form thereof, wherein the second agent is chosen from one or more of 1) a checkpoint modulator, 2) an XPO1 inhibitor, 3) an anti-CD19 antibody, 4) a TLR agonist, 5) a STING agonist, or 6) a Flt3 ligand, for a first period of time;
(b) after the first period of time, administering to the subject a therapeutically effective amount of a combination therapy comprising the PI3K inhibitor in combination with the second agent or a pharmaceutically acceptable form thereof; and
(c) optionally repeating steps (a) and (b) one or more times.

In certain embodiments, the subject is identified as developing resistance (e.g., acquired resistance) to the monotherapy.

In certain aspects, the disclosure provides a method of delaying or decreasing resistance of a subject having a cancer, comprising administering to the subject a synergistic amount of a PI3K inhibitor, or a pharmaceutically acceptable form thereof, and a second therapeutic agent selected from 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof. In a related aspect, provided herein is a composition for use in delaying or decreasing resistance of a subject having a cancer, said composition comprising a synergistic amount of a PI3K inhibitor, or a pharmaceutically acceptable form thereof, and a second therapeutic agent selected from 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof. In an embodiment, the resistance is resistance to the PI3K inhibitor. In an embodiment, the method comprises administering the PI3K inhibitor before the second therapeutic agent.

In some aspects, provided herein is a method of reducing the risk that a cancer becomes resistant to the PI3K inhibitor, comprising administering to a subject having a cancer a synergistic amount of a PI3K inhibitor, or a pharmaceutically acceptable form thereof, and a second therapeutic agent selected from one or more of 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof.

In some aspects, provided herein is a method of prolonging remission in a subject having a cancer, comprising administering to the subject a synergistic amount of a PI3K inhibitor, or a pharmaceutically acceptable form thereof, and a second therapeutic agent selected from 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof.

In some aspects, provided herein is a method of increasing the likelihood that a subject having a cancer experiences complete remission, comprising administering to the subject a synergistic amount of a PI3K inhibitor, or a pharmaceutically acceptable form thereof, and a second therapeutic agent selected from one or more of 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof.

In some aspects, provided herein is a method of reducing the level of minimal residual disease (MRD) in a subject having a cancer, comprising administering to the subject a synergistic amount of a PI3K inhibitor, or a pharmaceutically acceptable form thereof, and a second therapeutic agent selected from one or more of 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof. In another aspect, provided herein is a composition for use in reducing the level of minimal residual disease (MRD), said composition comprising a synergistic amount of a PI3K inhibitor, or a pharmaceutically acceptable form thereof, and a second therapeutic agent selected from one or more of 1) a checkpoint modulator, 2) an XPO1 inhibitor, 3) an anti-CD19 antibody, 4) a TLR agonist, 5) a STING agonist, or 6) a Flt3 ligand, or a pharmaceutically acceptable form thereof.

The disclosure includes all combinations of any one or more of the foregoing aspects and/or embodiments, as well as combinations with any one or more of the embodiments set forth in the detailed description and examples.

### INCORPORATION BY REFERENCE

All publications, patents, and patent applications mentioned in this specification are herein incorporated by reference in their entirety and to the same extent as if each individual publication, patent, or patent application is specifically and individually indicated to be incorporated by reference.

### BRIEF DESCRIPTION OF THE DRAWINGS

FIG. 1 shows an isobologram depicting the synergistic effect of the combination of Compound 1 and selinexor in H9 cell line.

### DETAILED DESCRIPTION

### 1. Definitions

Unless defined otherwise, all technical and scientific terms used herein have the same meaning as is commonly understood by one of skill in the art to which this specification pertains.

As used in the specification and claims, the singular form “a”, “an” and “the” includes plural references unless the context clearly dictates otherwise.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The term “agonist” as used herein refers to a compound or agent having the ability to initiate or enhance a biological function of a target protein or polypeptide, such as increasing the activity or expression of the target protein or polypeptide. Accordingly, the term “agonist” is defined in the context of the biological role of the target protein or polypeptide. While some agonists herein specifically interact with (e.g., bind to) the target, compounds and/or agents that initiate or enhance a biological activity of the target protein or polypeptide by interacting with other members of the signal transduction pathway of which the target polypeptide is a member are also specifically included within this definition.

The terms “antagonist” and “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. An inhibitor need not completely abrogate the biological function of a target protein or polypeptide, and in some embodiments reduces the activity by at least 50%, 60%, 70%, 80%, 90%, 95%, or 99%. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition. Non-limiting examples of biological activity inhibited by an antagonist include those associated with the development, growth, or spread of a tumor, or an undesired immune response as manifested in autoimmune disease.

The term “effective amount” or “therapeutically effective amount” refers to that amount of a compound or pharmaceutical composition described herein that is sufficient to effect the intended application including, but not limited to, disease treatment, as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on, for example, the particular compounds chosen, the dosing regimen to be followed, whether it is administered in combination with other agents, timing of administration, the tissue to which it is administered, and the physical delivery system in which it is carried.

As used herein, a daily dosage can be achieved by a single administration of the targeted dosage amount or multiple administrations of smaller dosage amount(s). For example, a 150 mg daily dosage can be achieved by a single administration of 150 mg of the therapeutic agent per day, two administrations of 75 mg of the therapeutic agent per day, or three administrations of 50 mg of the therapeutic agent per day, or the like.

As used herein, the terms “treatment” and “treating” are used herein to refer to an approach for obtaining beneficial or desired results including, but not limited to, therapeutic benefit. A therapeutic benefit includes, but is not limited to, eradication, inhibition, reduction, or amelioration of the underlying disorder being treated. Also, a therapeutic benefit is achieved with the eradication, inhibition, reduction, or amelioration of one or more of the physiological symptoms associated with the underlying disorder such that an improvement is observed in the patient, notwithstanding that the patient can still be afflicted with the underlying disorder.

As used herein, the terms “prevention” and “preventing” are used herein to refer to an approach for obtaining beneficial or desired results including, but not limited, to prophylactic benefit. For prophylactic benefit, the pharmaceutical compositions may be administered to a patient at risk of developing a particular disease, or to a patient reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” or “therapeutic agent” means a composition that will elicit the biological or medical response of a tissue, system, animal or human that is being sought by the researcher, veterinarian, medical doctor or other clinician.

As used herein, the “aggressiveness” of a tumor or cancer refers to the rate at which the tumor is growing. Thus, a tumor is more aggressive than another tumor or cancer if it is proliferating at a higher rate. Other determinants can be used to measure the level of aggressiveness of a tumor or cancer, for example, based on the appearance of tumor or cancer cells under a microscope to determine the extent to which tumors are differentiated. A well-differentiated tumor tends to be more aggressive than a poorly-differentiated tumor or cancer.

The term “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits one isoform of PI3K over another isoform of PI3K has an activity of at least greater than about 1× against a first isoform relative to the compound's activity against the second isoform (e.g., at least about 2×, 3×, 5×, 10×, 20×, 50×, 100×, 200×, 500×, or 1000×). In certain embodiments, these terms refer to (1) a compound described herein that selectively inhibits the gamma isoform over the alpha, beta, or delta isoform; or (2) a compound described herein that selectively inhibits the delta isoform over the alpha, beta, or gamma isoform. By way of non-limiting example, the ratio of selectivity can be greater than a factor of about 1, greater than a factor of about 2, greater than a factor of about 3, greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by IC₅₀. In certain embodiments, the IC₅₀ can be measured by in vitro or in vivo assays.

“Subject” or “patient” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

The term “in vivo” refers to an event that takes place in a subject's body.

The term “in vitro” refers to an event that takes places outside of a subject's body. For example, an in vitro assay encompasses any assay conducted outside of a subject In vitro assays encompass cell-based assays in which cells, alive or dead, are employed. In vitro assays also encompass a cell-free assay in which no intact cells are employed.

Combination therapy, or “in combination with” refer to the use of more than one compound or agent to treat a particular disorder or condition. For example, Compound 1 may be administered in combination with at least one additional therapeutic agent. By “in combination with,” it is not intended to imply that the other therapy and Compound 1 must be administered at the same time and/or formulated for delivery together, although these methods of delivery are within the scope of this disclosure. Compound 1 can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. In general, each therapeutic agent will be administered at a dose and/or on a time schedule determined for that particular agent. The other therapeutic agent can be administered with Compound 1 herein in a single composition or separately in a different composition. Higher combinations, e.g., triple therapy, are also contemplated herein.

The terms “co-administration of” and “co-administering” and their grammatical equivalents, as used herein, encompass administration of two or more agents to subject so that both agents and/or their metabolites are present in the subject at the same or substantially the same time. In one embodiment, co-administration of a PI3K inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the PI3K inhibitor, or in some combination thereof. Where the PI3K inhibitor is administered to the patient at repeated intervals, e.g., during a standard course of treatment, the additional agent can be administered prior to, at the same time as, or subsequent to, each administration of the PI3K inhibitor, or some combination thereof, or at different intervals in relation to the PI3K inhibitor treatment, or in a single dose prior to, at any time during, or subsequent to the course of treatment with the PI3K inhibitor. In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “monotherapy” refers to the use of an agent individually (also referred to herein as alone) (e.g., as a single compound or agent), e.g., without a second active ingredient to treat the same indication, e.g., cancer. For example, in this context, the term monotherapy includes the use of either the PI3K inhibitor or the second agent individually to treat the cancer.

The term “synergy” or “synergistic” encompasses a more than additive effect of a combination of two or more agents compared to their individual effects. In certain embodiments, synergy or synergistic effect refers to an advantageous effect of using two or more agents in combination, e.g., in a pharmaceutical composition, or in a method of treatment. In certain embodiments, one or more advantageous effects is achieved by using a PI3K inhibitor in combination with a second therapeutic agent (e.g., one or more second therapeutic agents) as described herein.

In some embodiments, the synergistic effect is that a lower dosage of one or both of the agents is needed to achieve an effect. For example, the combination can provide a selected effect, e.g., a therapeutic effect, when at least one of the agents is administered at a lower dosage than the dose of that agent that would be required to achieve the same therapeutic effect when the agent is administered as a monotherapy. In certain embodiments, the combination of a PI3K inhibitor (e.g., Compound 1) and a second agent (as described herein) allows the PI3K inhibitor to be administered at a lower dosage than would be required to achieve the same therapeutic effect if the PI3K inhibitor were administered as a monotherapy.

In some embodiments, the synergistic effect is a reduction, prevention, delay, or decrease in the occurrence or the likelihood of occurrence of one or more side effects, toxicity, resistance, that would otherwise be associated with administration of at least one of the agents.

In some embodiments, the synergistic effect is a reduction in resistance (e.g., a decrease in a measure of resistance or a decreased likelihood of developing resistance), or a delay in the development of resistance, to at least one of the agents.

In some embodiments, the synergistic effect is a reduction in minimal residual disease (MRD). In certain embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination was administered). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent. In certain embodiments, the MRD is decreased below the level observed during treatment with a monotherapy comprising the PI3K inhibitor. In certain embodiments, the MRD is decreased below the level observed during treatment with a monotherapy comprising the second agent. In certain embodiments, the combination is effective to reduce the level of MRD below a preselected cutoff value (e.g., 1 malignant cell in 100 normal cells, 1 malignant cell in 1000 normal cells, or 1 malignant cell in 10,000 normal cells, or 1 malignant cell in 100,000 normal cells). In certain embodiments, the preselected cutoff value is 1 malignant cell in 1000 normal cells. In certain embodiments, the preselected cutoff value is 1 malignant cell in 100,000 normal cells.

In some embodiments, a synergistic effect refers to the combination of a PI3K inhibitor (e.g., Compound 1, or a pharmaceutically acceptable form thereof), and a second therapeutic agent (e.g., one or more additional therapeutic agent(s), or a pharmaceutically acceptable form thereof, as described herein), results in a therapeutic effect greater than the additive effect of the PI3K inhibitor and the second agent.

In some embodiments, a synergistic effect means that combination index value is less than a selected value, e.g., for a given effect, e.g., at a selected percentage (e.g., 50%) inhibition or growth inhibition, e.g., as described herein in the Examples. In certain embodiments, the selected value is 1. In certain embodiments, the selected value is 0.7. In certain embodiments, the selected value is 0.5.

In some embodiments, a synergistic effect means that the synergy score is 1 or more. In certain embodiments, the synergy score is greater than 1. In certain embodiments, the synergy score is greater than 2. In certain embodiments, the synergy score is greater than 3.

Combination index (CI) is a measure of potency shifting. The combination index is known in the art and is described, e.g., in Chou et al., Adv Enzyme Regul 1984: 22: 27-55 and in U.S. Patent Publication No. 2013/0295102, the contents of which are incorporated herein by reference. A CI value of greater than 1 indicates antagonistic effect; a CI value of 1.0 is indicative of an additive effect; and a CI value of less than 1 is indicative of a synergistic effect resulting from the combination. The CI value can be determined at various percentages of inhibition or growth inhibition.

The CI provides an estimate of the fraction of the original (monotherapy) doses of each of two drugs would be needed in combination relative to the single agent doses required to achieve a chosen effect level. For example, when the combination index has a value of 0.1, only about one tenth of the total fractional amounts of the individual agents (expressed as a fraction of the amount of that agent when administered as a monotherapy to achieve a chosen effect) are needed for the combination to reach the same chosen effect level. For example, if a dose of 100 mg/kg of drug A individually or a dose of 200 mg/kg of drug B individually is needed to achieve the chosen effect, and the combination index is 0.1, then approximately 5 mg/kg of drug A and 10 mg/kg of drug B would achieve the chosen effect (one twentieth of the original doses of each of the single agents adds up to a total of one tenth). The doses of the single agents need not be reduced by the same fractional value so long as the sum of their fractional values adds up to the combination index; thus, in this example, a dose of approximately 8 mg/kg of drug A and 4 mg/kg of drug B would also achieve the chosen effect (this is 0.08 times the original dose of drug A and 0.02 times the original dose of drug B; the sum of the fractional amounts (0.08+0.02) is equal to the combination index of 0.1.)

According to one embodiment, synergy score is a measure of the combination effects in excess of Loewe additivity. In one example, synergy score is a scalar measure to characterize the strength of synergistic interaction. The Synergy score can be calculated as:

$$Synergy Score = log {f}_{X} log {f}_{Y} Σ max ( 0 , {I}_{d a t a} ) ( {I}_{d a t a} - {I}_{L o e w e} )$$

In this example, the fractional inhibition for each component agent and combination point in the matrix is calculated relative to the median of all vehicle-treated control wells. The example Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels. According to other embodiments, a synergy score can be calculated based on a curve fitting approach where the curvature of the synergy score is extrapolated by introducing a median value and origin value (e.g., a dose zero value).

The synergy score measure can be used for the self-cross analysis. Synergy scores of self-crosses are expected to be additive by definition and, therefore, maintain a synergy score of zero. However, while some self-cross synergy scores are near zero, many are greater suggesting that experimental noise or non-optimal curve fitting of the single agent dose responses are contributing to the slight perturbations in the score. This strategy is cell line-centric, focusing on self-cross behavior in each cell line versus a global review of cell line panel activity. Combinations where the synergy score is greater than the mean self-cross plus two standard deviations or three standard deviations can be considered candidate synergies at 95% and 99% confidence levels, respectively. Additivity should maintain a synergy score of zero, and synergy score of two or three standard deviations indicate synergism at statistically significant levels of 95% and 99%.

Loewe Volume (Loewe Vol) is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

$${I}_{L o e w e} that satisfies ( X / {X}_{I} ) + ( Y / {Y}_{I} ) = 1$$

where X₁ and Y₁ are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

The term “anti-cancer effect” refers to the effect a therapeutic agent has on cancer, e.g., a decrease in growth, viability, or both of a cancer cell. The IC₅₀ of cancer cells can be used as a measure the anti-cancer effect.

IC₅₀ refers to a measure of the effectiveness of a therapeutic agent in inhibiting cancer cells by 50%.

The term “tumor” refers to any neoplastic cell growth and proliferation, whether malignant or benign, and any pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus.

Hematopoietic origin refers to involving cells generated during hematopoiesis, a process by which cellular elements of blood, such as lymphocytes, leukocytes, platelets, erythrocytes and natural killer cells are generated. Cancers of hematopoietic origin includes lymphoma and leukemia.

Resistant or refractive refers to when a cancer that has a reduced responsiveness to a treatment, e.g., up to the point where the cancer does not respond to treatment. The cancer can be resistant at the beginning of treatment, or it may become resistant during treatment. The cancer subject may have one or more mutations that cause it to become resistant to the treatment, or the subject may have developed such mutations during treatment. The term “refractory” can refer to a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

“Responsiveness,” to “respond” to treatment, and other forms of this term, as used herein, refer to the reaction of a subject to treatment with a therapeutic, e.g., a PI3K inhibitor, alone or in combination, e.g., monotherapy or combination therapy. In one embodiment, a response to a PI3K inhibitor is determined. Responsiveness to a therapy, e.g., treatment with a PI3K inhibitor alone or in combination, can be evaluated by using any of the alterations/biomarkers disclosed herein and/or comparing a subject's response to the therapy using one or more clinical criteria, such as IWCLL 2008 (for CLL) described in, e.g., Hallek, M. et al. (2008) Blood 111 (12): 5446-5456; RECIST criteria for solid tumors (Response Evaluation Criteria In Solid Tumors), and the like. Additional classifications of responsiveness are provided in Brown, J. R. (2014) Blood, 123(22):3390-3397 and Chesson, B. D. et al. Journal of Clinical Oncology, 30(23):2820-2822.

These criteria provide a set of published rules that define when cancer patients improve (“respond”), stay the same (“stable”) or worsen (“progression”) during treatments.

In one embodiment, a subject having CLL can be determined to be in complete remission (CR) or partial remission (PR). For example, according to IWCLL 2008, a subject is considered to be in CR if at least all of the following criteria as assessed after completion of therapy are met: (i) Peripheral blood lymphocytes (evaluated by blood and different count) below 4×10⁹/L (4000 μL); (ii) no hepatomegaly or splenomegaly by physical examination; (iii) absence of constitutional symptoms; and (iv) blood counts (e.g., neutrophils, platelets, hemoglobin) above the values set forth in Hallek, M. et al. supra at page 5451). Partial remission (PR) for CLL is defined according to IWCLL 2008 as including one of: (i) a decrease in number of blood lymphocytes by 50% or more from the value before therapy; (ii) a reduction in lymphadenopathy, as detected by CT scan or palpation; or (iii) a reduction in pretreatment enlargement of spleen or liver by 50% or more, as detected by CT scan or palpation; and blood counts (e.g., neutrophils, platelets, hemoglobin) according to the values set forth in Hallek, M. et al. supra at page 5451).

In other embodiments, a subject having CLL is determined to have progressive disease (PD) or stable disease (SD). For example, according to IWCLL 2008, a subject is considered to be in PD during therapy or after therapy if at least one of the following criteria is met: (i) progression on lymphadenopathy; (ii) an increase in pretreatment enlargement of spleen or liver by 50% or more, or de novo appearance of hepatomegaly or splenomegaly; (iii) an increase in the number of blood lymphocytes by 50% or more with at least 5000 B lymphocytes per microliter; (iv) transformation to a more aggressive histology (e.g., Richter syndrome); or (v) occurrence of cytopenia (neutropenia, anemia or thrombocytopenia) attributable to CLL, as described in Hallek, M. et al. supra at page 5452. Stable disease (SD) for CLL is defined according to IWCLL 2008 as a patient who has not achieved CR or a PR, and who has not exhibited progressive disease, see Hallek, M. et al. supra at page 5452.

In one embodiment, a subject with CLL responds to treatment with an PI3K inhibitor if at least one of the criteria for disease progression according to IWCLL is retarded or reduced, e.g., by about 10%, 20%, 30%, 40%, 50%, 60%, 70%, 80%, 90% or more. In another example, a subject responds to treatment with a PI3K inhibitor, if the subject experiences a life expectancy extension, e.g., extended by about 5%, 10%, 20%, 30%, 40%, 50% or more beyond the life expectancy predicted if no treatment is administered. In another example, a subject responds to treatment with a PI3K inhibitor, if the subject has one or more of: an increased progression-free survival, overall survival or increased time to progression (TTP), e.g., as described in Hallek, M. et al. supra at page 5452.

In another embodiment in solid tumors, a subject responds to treatment with a PI3K inhibitor if growth of a tumor in the subject is retarded about 10%, 20%, 30%, 40%, 50%, 60%, 70%, 80%, 90% or more. In another example, a subject responds to treatment with a PI3K inhibitor, if a tumor in the subject shrinks by about 5%, 10%, 20%, 30%, 40%, 50% or more as determined by any appropriate measure, e.g., by mass or volume. In another example, a subject responds to treatment with a PI3K inhibitor, if the subject experiences a life expectancy extended by about 5%, 10%, 20%, 30%, 40%, 50% or more beyond the life expectancy predicted if no treatment is administered. In another example, a subject responds to treatment with a PI3K inhibitor, if the subject has an increased disease-free survival, overall survival or increased time to progression. Several methods can be used to determine if a patient responds to a treatment including the RECIST criteria, as set forth above.

“Acquire” or “acquiring” as the terms are used herein, refer to obtaining possession of, determining, or evaluating, a value or information (e.g., one or more of: the presence, absence, amount or level) of an alteration or biomarker, by “directly acquiring” or “indirectly acquiring” the same. “Directly acquiring” means performing a process (e.g., performing a test) to obtain the value or information of the alteration or biomarker. “Indirectly acquiring” refers to receiving the value or information of the alteration or biomarker from another party or source (e.g., a diagnostic provider, a third party clinician or health professional).

### Chemical Definitions

As used herein, a “pharmaceutically acceptable form” of a disclosed compound includes, but is not limited to, pharmaceutically acceptable salts, hydrates, solvates, isomers, prodrugs, and isotopically labeled derivatives of disclosed compounds. In one embodiment, a “pharmaceutically acceptable form” includes, but is not limited to, pharmaceutically acceptable salts, isomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts may be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts may be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the pharmaceutically acceptable form is a solvate (e.g., a hydrate). As used herein, the term “solvate” refers to compounds that further include a stoichiometric or non-stoichiometric amount of solvent bound by non-covalent intermolecular forces. The solvate may be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or one to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” refers to compounds that are transformed in vivo to yield a disclosed compound or a pharmaceutically acceptable form of the compound. A prodrug may be inactive when administered to a subject, but is converted in vivo to an active compound, for example, by hydrolysis (e.g., hydrolysis in blood). In certain cases, a prodrug has improved physical and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, Chp 1, pp 1-12 and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it enhances absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elsevier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as P-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a disclosed compound or a pharmaceutically acceptable form of the compound contains an alcohol functional group, a prodrug may be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from naturally occurring L-amino acids, P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug may be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural α-aminoacyl or natural α-aminoacyl-natural α-aminoacyl, —C(OH)C(O)OY¹ wherein Y¹ is H, (C₁-C₆)alkyl or benzyl, —C(0Y²)Y³ wherein Y² is (C₁-C₄) alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino, morpholino, piperidin-1-yl or pyrrolidin-1-yl.

In certain embodiments, the pharmaceutically acceptable form is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof, and other mixtures thereof, as falling within the scope of this disclosure.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown may be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that may be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which can potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound may be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

In certain embodiments, the pharmaceutically acceptable form is a tautomer. As used herein, the term “tautomer” is a type of isomer that includes two or more interconvertable compounds resulting from at least one formal migration of a hydrogen atom and at least one change in valency (e.g., a single bond to a double bond, a triple bond to a double bond, or a triple bond to a single bond, or vice versa). “Tautomerization” includes prototropic or proton-shift tautomerization, which is considered a subset of acid-base chemistry. “Prototropic tautomerization” or “proton-shift tautomerization” involves the migration of a proton accompanied by changes in bond order. The exact ratio of the tautomers depends on several factors, including temperature, solvent, and pH. Where tautomerization is possible (e.g., in solution), a chemical equilibrium of tautomers may be reached. Tautomerizations (i.e., the reaction providing a tautomeric pair) may be catalyzed by acid or base, or can occur without the action or presence of an external agent. Exemplary tautomerizations include, but are not limited to, keto-enol; amide-imide; lactam-lactim; enamine-imine; and enamine-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

The disclosure also embraces isotopically labeled compounds which are identical to those recited herein, except that one or more atoms are replaced by an atom having an atomic mass or mass number different from the atomic mass or mass number usually found in nature. Examples of isotopes that may be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ⁵N, ¹⁸O, ¹⁷O ³¹P, ³²p ³⁵S, ⁸F, and ³⁶Cl, respectively. Certain isotopically-labeled disclosed compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

As used herein, and unless otherwise specified, “polymorph” may be used herein to describe a crystalline material, e.g., a crystalline form. In certain embodiments, “polymorph” as used herein are also meant to include all crystalline and amorphous forms of a compound or a salt thereof, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms, as well as mixtures thereof, unless a particular crystalline or amorphous form is referred to. Compounds of the present disclosure include crystalline and amorphous forms of those compounds, including, for example, crystalline forms, polymorphs, pseudopolymorphs, solvates, hydrates, co-crystals, unsolvated polymorphs (including anhydrates), conformational polymorphs, tautomeric forms, disordered crystalline forms, and amorphous forms of the compounds or a salt thereof, as well as mixtures thereof.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as disclosed herein is contemplated. Supplementary active ingredients can also be incorporated into the pharmaceutical compositions.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### 2. Compositions and Methods

In the methods and compositions described herein, the PI3K inhibitor can be any PI3K inhibitor as described herein below, including pharmacologically acceptable salts or polymorphs thereof.

As used herein, a “phosphoinositide 3-kinase (PI3K) inhibitor” or “PI3K inhibitor” refers to an inhibitor of any PI3K. PI3Ks are members of a unique and conserved family of intracellular lipid kinases that phosphorylate the 3′—OH group on phosphatidylinositols or phosphoinositides. The PI3K family includes kinases with distinct substrate specificities, expression patterns, and modes of regulation (see, e.g., Katso et al., 2001, Annu. Rev. Cell Dev. Biol. 17, 615-675; Foster, F. M. et al., 2003, JCell Sci 116, 3037-3040). The class I PI3Ks (e.g., p110 α, p110 β, p110 γ, and p110 δ) are typically activated by tyrosine kinases or G-protein coupled receptors to generate PIP3, which engages downstream mediators such as those in the Akt/PDK1 pathway, mTOR, the Tec family kinases, and the Rho family GTPases. The class II PI3Ks (e.g., PI3K-C2a, PI3K-C2p, PI3K-C2γ) and III PI3Ks (e.g., Vps34) play a key role in intracellular trafficking through the synthesis of PI(3)P and PI(3,4)P2. Specific exemplary PI3K inhibitors are disclosed herein.

The class I PI3Ks comprise a p110 catalytic subunit and a regulatory adapter subunit. See, e.g., Cantrell, D. A. (2001) Journal of Cell Science 114: 1439-1445. Four isoforms of the p110 subunit (including PI3K-α (alpha), PI3K-β (beta), PI3K-γ (gamma), and PI3K-δ (delta) isoforms) have been implicated in various biological functions. Class I PI3Kα is involved, for example, in insulin signaling, and has been found to be mutated in solid tumors. Class I PI3K-β is involved, for example, in platelet activation and insulin signaling. Class I PI3K-γ plays a role in mast cell activation, innate immune function, and immune cell trafficking (chemokines). Class I PI3K-δ is involved, for example, in B-cell and T-cell activation and function and in Fc receptor signaling in mast cells. In some embodiments provided herein, the PI3K inhibitor is a class I PI3K inhibitor. In some such embodiments, the PI3K inhibitor inhibits a PI3K-α (alpha), PI3K-β(beta), PI3K-γ (gamma), or PI3K-δ (delta) isoform, or a combination thereof.

Downstream mediators of the PI3K signal transduction pathway include Akt and mammalian target of rapamycin (mTOR). Manning et al., Cell 129, 1261-1274 Jun. 29, 2007. Akt possesses a plckstrin homology (PH) domain that binds PIP3, leading to Akt kinase activation. Akt phosphorylates many substrates and is a central downstream effector of PI3K for diverse cellular responses. One important function of Akt is to augment the activity of mTOR, through phosphorylation of TSC2 and other mechanisms. mTOR is a serine-threonine kinase related to the lipid kinases of the PI3K family. Laplante et al., Cell 149,274-293 Apr. 13, 2012 mTOR has been implicated in a wide range of biological processes including cell growth, cell proliferation, cell motility and survival. Disregulation of the mTOR pathway has been reported in various types of cancer. mTOR is a multifunctional kinase that integrates growth factor and nutrient signals to regulate protein translation, nutrient uptake, autophagy, and mitochondrial function.

In certain embodiments, provided herein are pharmaceutical compositions comprising a PI3K inhibitor, or a pharmaceutically acceptable form thereof, in combination with a second agent or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand. In certain embodiments, the combination is therapeutically effective. In certain embodiments, the combination is synergistic, e.g., has one or more synergistic effects, e.g., synergistic therapeutic effects.

Also provided herein are methods of treating, managing, or preventing a cancer in a subject comprising administering to the subject a PI3K inhibitor, or a pharmaceutically acceptable form thereof, in combination with a second agent (e.g., one or more second agents), or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand. In certain embodiments, the combination is therapeutically effective. In certain embodiments, the combination is synergistic, e.g., has one or more synergistic effects, e.g., synergistic therapeutic effects.

In certain embodiments, the compositions and methods provided herein are utilized where a monotherapy of one of the therapeutic agents is becoming less effective due to drug resistance or where the relatively high dosage of monotherapy lead to undesirable side effects.

### 2.1 PI3K Inhibitors

PI3K inhibitors that can be used in the compositions and methods provided herein include, but are not limited to, those described in, e.g., WO 09/088990, WO 09/088086, WO 2011/008302, WO 2010/036380, WO 2010/006086, WO 09/114870, WO 05/113556, WO2014072937, WO2014071125, US 2009/0312310, and US 2011/0046165, the entirety of each incorporated herein by reference. Additional PI3K inhibitors that can be used in the compositions and methods provided herein include, but are not limited to, AMG-319, GSK 2126458 (2,4-Difluoro-N-{2-(methyloxy)-5-[4-(4-pyridazinyl)-6-quinolinyl]-3-pyridinyl}benzenesulfonamide), GSK 1059615 (5Z-[[4-(4-pyridinyl)-6-quinolinyl]methylene]-2,4-thiazolidinedione), GDC-0032 (4-[5,6-dihydro-2-[3-methyl-1-(1-methylethyl)-1H-1,2,4-triazol-5-yl]imidazo[1,2-d][1,4]benzoxazepin-9-yl]-α,α-dimethyl-1H-Pyrazole-1-acetamide), GDC-0980 ((S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one), GDC-0941 (2-(1H-indazol-4-yl)-6-((4-(methylsulfonyl)piperazin-1-yl)methyl)-4-morpholinothieno[3,2-d]pyrimidine), XL147 (N-(3-(benzo[c][1,2,5]thiadiazol-5-ylamino)quinoxalin-2-yl)-4-methylbenzenesulfonamide), XL499, XL765 (SAR245409, N-[4-[[[3-[(3,5-dimethoxyphenyl)amino]-2-quinoxalinyl]amino]sulfonyl]phenyl]-3-methoxy-4-methyl-benzamide), PF-4691502 (2-amino-6-(6-methoxypyridin-3-yl)-4-methyl-8-[(1R,4R)-4-(2-hydroxyethoxy)cyclohexyl]-7H,8H-pyrido[2,3-d]pyrimidin-7-one), BKM 120 (buparlisib, 5-(2,6-dimorpholinopyrimidin-4-yl)-4-(trifluoromethyl)pyridin-2-amine), Idelalisib (CAL-101, GS1101, (S)-2-(1-(9H-purin-6-ylamino)propyl)-5-fluoro-3-phenylquinazolin-4(3H)-one), CAL 263, SF1126 (3-[[2-[[5-[[amino(azaniumyl)methylidene]amino]-2-[[4-oxo-4-[4-(4-oxo-8-phenylchromen-2-yl)morpholin-4-ium-4-yl]oxybutanoyl]amino]pentanoyl]amino]acetyl]amino]-4-(1-carboxylatopropylamino)-4-oxobutanoate), PX-866 (sonolisib, [(3aR,6E,9S,9aR,10R,11aS)-6-[[bis(prop-2-enyl)amino]methylidene]-5-hydroxy-9-(methoxymethyl)-9a, l1α-dimethyl-1,4,7-trioxo-2,3,3a,9,10,11-hexahydroindeno[4,5-h]isochromen-10-yl]acetate), BEZ235 (2-methyl-2-(4-(3-methyl-2-oxo-8-(quinolin-3-yl)-2,3-dihydroimidazo[4,5-c]quinolin-1-yl)phenyl)propanenitrile), GS9820 (CAL-120, (S)-2-(1-((9H-purin-6-yl)amino)ethyl)-6-fluoro-3-phenylquinazolin-4(3H)-one), BYL719 ((2S)-1,2-Pyrrolidinedicarboxamide, N1-[4-methyl-5-[2-(2,2,2-trifluoro-1,1-dimethylethyl)-4-pyridinyl]-2-thiazolyl]), RP6503, RP6530, TGR1202 (((S)-2-(1-(4-amino-3-(3-fluoro-4-isopropoxyphenyl)-1H-pyrazolo[3,4-d]pyrimidin--yl)ethyl)-6-fluoro-3-(3-fluorophenyl)-4H-chromen-4-one)), INK1117 (MLN-1117), PX-866, BAY 80-6946 (2-amino-N-(7-methoxy-8-(3-morpholinopropoxy)-2,3-dihydroimidazo[1,2-c]quinazolin-5-yl)pyrimidine-5-carboxamide), IC87114 (2-((6-amino-9H-purin-9-yl)methyl)-5-methyl-3-o-tolylquinazolin-4(3H)-one), Palomid 529 (3-(4-methoxybenzyloxy)-8-(1-hydroxyethyl)-2-methoxy-6H-benzo[c]chromen-6-one), ZSTK474 (2-(difluoromethyl)-1-(4,6-dimorpholino-1,3,5-triazin-2-yl)-1H-benzo[d]imidazole), PWT33597, TG100-115 (6,7-Bis(3-hydroxyphenyl)pteridine-2,4-diamine), GNE-477 (5-[7-methyl-4-(morpholin-4-yl)-6-[(4-methylsulfonylpiperazin-1-yl)methyl]thieno[3,2-d]pyrimidin-2-yl]pyrimidin-2-amine), CUDC-907 (N-hydroxy-2-(((2-(6-methoxypyridin-3-yl)-4-morpholinothieno[3,2-d]pyrimidin-6-yl)methyl)(methyl)amino)pyrimidine-5-carboxamide), AEZS-136, BGT-226 (8-(6-methoxypyridin-3-yl)-3-methyl-1-(4-(piperazin-1-yl)-3-(trifluoromethyl)phenyl)-1H-imidazo[4,5-c]quinolin-2(3H)-one maleic acid), PF-05212384 (1-(4-(4-(dimethylamino)piperidine-1-carbonyl)phenyl)-3-(4-(4,6-dimorpholino-1,3,5-triazin-2-yl)phenyl)urea), LY3023414, PI-103 (3-[4-(4-morpholinyl)pyrido[3′,2′:4,5]furo[3,2-d]pyrimidin-2-yl]-phenol), INCB040093, CAL-130 ((S)-2-(1-((2-amino-9H-purin-6-yl)amino)ethyl)-5-methyl-3-(o-tolyl)quinazolin-4(3H)-one), LY294002 (2-Morpholin-4-yl-8-phenylchromen-4-one) and wortmannin.

In one embodiment, the PI3K inhibitor is Idelalisib (GS1101), CAL-130, BKM 120, GDC-0941, PX-866, GDC-0032, BAY 80-6946, BEZ235, BYL719, BGT-226, PF-4691502, GDC-0980, GSK 2126458, PF-05212384, XL765, or XL147.

In one embodiment, the PI3K inhibitor is Idelalisib (also known as GS1101 or CAL-101) and has the chemical name (S)-2-(1-(9H-purin-6-ylamino)propyl)-5-fluoro-3-phenylquinazolin-4(3H)-one and the following structure:

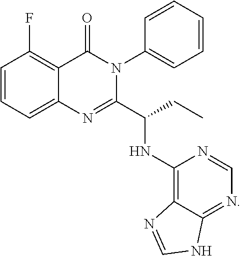 [SMILES: CC[C@H](Nc1ncnc2[nH]cnc12)c1nc2cccc(F)c2c(=O)n1-c1ccccc1]

In certain embodiments, a PI3K inhibitor is a compound that inhibits one or more PI3K isoforms, e.g., alpha, beta, delta, or gamma isoform. In one embodiment, a PI3K inhibitor is a compound that inhibits one or more PI3K isoforms with an IC₅₀ of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 10 nM, less than about 5 nM, or less than about 1 nM.

In one embodiment, the PI3K inhibitor is a compound that inhibits alpha, beta, delta and gamma isoforms of PI3K. In another embodiment, the PI3K inhibitor is a compound that inhibits beta, delta, and gamma isoforms of PI3K. In another embodiment, the PI3K inhibitor is a compound that inhibits the delta and gamma isoforms of PI3K.

In certain embodiments, the PI3K inhibitor is a PI3K isoform selective inhibitor. In one embodiment, the PI3K inhibitor is a PI3K alpha selective inhibitor. In another embodiment, the PI3K inhibitor is a PI3K beta selective inhibitor.

In certain embodiments, the PI3K inhibitor is a PI3K delta selective inhibitor. In one embodiment, the PI3K delta selective inhibitor selectively inhibits PI3K delta isoform over PI3K gamma isoform. In one embodiment, the PI3K delta selective inhibitor has a gamma/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a gamma/delta selectivity ratio in the range of from greater than 1 to about 5, from about 5 to about 10, from about 10 to about 50, from about 50 to about 850, or greater than about 850. In one embodiment, the gamma/delta selectivity ratio is determined by dividing the inhibitor's IC₅₀ against PI3K gamma isoform by the inhibitor's IC₅₀ against PI3K delta isoform.

In certain embodiments, the PI3K inhibitor is a PI3K delta selective inhibitor. In one embodiment, the PI3K delta selective inhibitor selectively inhibits PI3K delta isoform over PI3K alpha isoform. In one embodiment, the PI3K delta selective inhibitor has an alpha/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has an alpha/delta selectivity ratio in the range of from greater than 1 to about 5, from about 5 to about 10, from about 10 to about 50, from about 50 to about 850, or greater than about 850. In one embodiment, the alpha/delta selectivity ratio is determined by dividing the inhibitor's IC₅₀ against PI3K alpha isoform by the inhibitor's IC₅₀ against PI3K delta isoform.

In certain embodiments, the PI3K inhibitor is a PI3K delta selective inhibitor. In one embodiment, the PI3K delta selective inhibitor selectively inhibits PI3K delta isoform over PI3K beta isoform. In one embodiment, the PI3K delta selective inhibitor has a beta/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a beta/delta selectivity ratio in the range of from greater than 1 to about 5, from about 5 to about 10, from about 10 to about 50, from about 50 to about 850, or greater than about 850. In one embodiment, the beta/delta selectivity ratio is determined by dividing the inhibitor's IC₅₀ against PI3K beta isoform by the inhibitor's IC₅₀ against PI3K delta isoform.

In certain embodiments, the PI3K inhibitor is selective for both gamma and delta. In one embodiment, the PI3K gamma and delta selective inhibitor selectively inhibits PI3K gamma and delta isoforms over PI3K beta isoform. In one embodiment, the PI3K gamma and delta selective inhibitor has a beta/delta selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000 and a beta/gamma selectivity ratio of greater than 1, greater than about 5, greater than about 10, greater than about 50, greater than about 100, greater than about 200, greater than about 400, greater than about 600, greater than about 800, greater than about 1000, greater than about 1500, greater than about 2000, greater than about 5000, greater than about 10,000, or greater than about 20,000. In one embodiment, the PI3K delta selective inhibitor has a beta/delta selectivity ratio in the range of from greater than 1 to about 5, from about 5 to about 10, from about 10 to about 50, from about 50 to about 850, or greater than about 850 and a beta/gamma selectivity ratio in the range of from greater than 1 to about 5, from about 5 to about 10, from about 10 to about 50, from about 50 to about 850, or greater than about 850. In one embodiment, the beta/delta selectivity ratio is determined by dividing the inhibitor's IC50 against PI3K beta isoform by the inhibitor's IC50 against PI3K delta isoform and the beta/gamma selectivity ratio is determined by dividing the inhibitor's IC50 against PI3K beta isoform by the inhibitor's IC50 against PI3K gamma isoform.

PI3K delta inhibitors that can be used in the compositions and methods provided herein include, but are not limited to, GSK-2269557 (2-(6-(1H-indol-4-yl)-1H-indazol-4-yl)-5-((4-isopropylpiperazin-1-yl)methyl)oxazole), GS-9820, GS-1101 (5-fluoro-3-phenyl-2-([S)]-1-[9H-purin-6-ylamino]-propyl)-3H-quinazolin-4-one), AMG319, or TGR-1202 (((S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one)), or a mixture thereof. In one embodiment, the PI3K delta inhibitor is Idelalisib.

In one embodiment, the PI3K inhibitor is a PI3K inhibitor as described in WO 2005/113556, the entirety of which is incorporated herein by reference. In one embodiment, the PI3K inhibitor is Compound Nos. 113 or 107 as described in WO2005/113556.

In one embodiment, the PI3K inhibitor is a PI3K inhibitor as described in WO2014/006572, the entirety of which is incorporated herein by reference. In one embodiment, the PI3K inhibitor is Compound Nos. A1, A2, B, B1, or B2 as described in WO2014/006572.

In certain embodiments, the PI3K inhibitor is a PI3K delta/gamma dual inhibitor. In one embodiment, the PI3K delta/gamma dual inhibitor has an IC₅₀ value against PI3K alpha that is at least 5×, 10×, 20×, 50×, 100×, 200×, 500×, or 1000× higher than its IC₅₀ values against delta and gamma.

In certain embodiments, the PI3K inhibitor is Compound 1 of the structure:

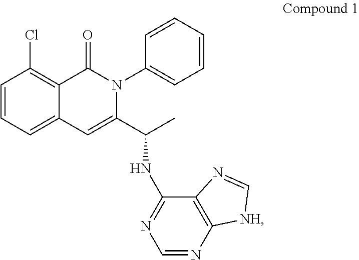 [SMILES: C[C@H](Nc1ncnc2[nH]cnc12)c1cc2cccc(Cl)c2c(=O)n1-c1ccccc1]

or a pharmaceutically acceptable form thereof.

Compound 1 has a chemical name of (S)-3-(1-((9H-purin-6-yl)amino)ethyl)-8-chloro-2-phenylisoquinolin-1(2H)-one. An exemplary method for synthesizing Compound 1 has been previously described in U.S. Pat. No. 8,193,182, which is incorporated by reference in its entirety. Without being limited by a particular theory, Compound 1 is a PI3K delta/gamma dual inhibitor and can be used to treat cancers. See U.S. Pat. No. 8,193,182.

Compound 1 provided herein contains one chiral center, and can exist as a mixture of enantiomers, e.g., a racemic mixture. This application encompasses the use of stereomerically pure forms of such a compound, as well as the use of mixtures of those forms. For example, mixtures comprising equal or unequal amounts of the enantiomers of Compound 1 provided herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables ofResolving Agents and Optical Resolutions p. 268 (E. L. Eliel, Ed., Univ. of Notre Dame Press, Notre Dame, I N, 1972).

In one embodiment, the PI3K inhibitor provided herein is a mixture of Compound 1 and its (R)-enantiomer. In one embodiment, the PI3K inhibitor provided herein is a racemic mixture of Compound 1 and its (R)-enantiomer. In other embodiments, the compound mixture has an (S)-enantiomeric purity of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (R)-enantiomeric purity of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

As used herein, Compound 1 also refers to any crystal form or polymorph of (S)-3-(1-((9H-purin-6-yl)amino)ethyl)-8-chloro-2-phenylisoquinolin-1(2H)-one. In some embodiments, a polymorph of Compound 1, or a pharmaceutically form thereof, disclosed herein is used. Exemplary polymorphs are disclosed in U.S. Patent Publication No. 2012/0184568, which is hereby incorporated by reference in its entirety. In one embodiment, the compound is Form A of Compound 1. In one embodiment, the compound is Form B of Compound 1. In one embodiment, the compound is Form C of Compound 1. In one embodiment, the compound is Form D of Compound 1. In one embodiment, the compound is Form E of Compound 1. In one embodiment, the compound is Form F of Compound 1. In one embodiment, the compound is Form G of Compound 1. In one embodiment, the compound is Form H of Compound 1. In one embodiment, the compound is Form I of Compound 1. In one embodiment, the compound is Form J of Compound 1. In one embodiment, the compound is a mixture of solid forms (e.g., polymorphs and/or amorphous forms) of Compound 1 disclosed herein.

In one embodiment, the composition comprises the PI3K delta selective inhibitor (e.g. Idelalisib), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta selective inhibitor (e.g.GS1101), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta selective inhibitor which is Idelalisib, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the PI3K delta inhibitor (e.g., Idelalisib) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 6000 ng/mL*hr to about 8000 ng/mL*hr, about 6500 ng/mL*hr to about 7500 ng/mL*hr, or about 7000 ng/mL*hr.

In one embodiment, the PI3K delta inhibitor (e.g., Idelalisib) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the PI3K delta inhibitor (e.g., Idelalisib) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2300 ng/mL, about 2000 ng/mL to about 2300 ng/mL, or about 2200 ng/mL.

In one embodiment, the PI3K delta inhibitor (e.g., Idelalisib) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the composition comprises the PI3K delta inhibitor (e.g., Idelalisib), or a pharmaceutically acceptable form thereof, at an amount in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., Idelalisib), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta inhibitor (e.g., Idelalisib), or a pharmaceutically acceptable form thereof, at an amount of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In one embodiment, the PI3K delta inhibitor (e.g., Idelalisib), or a pharmaceutically acceptable form thereof, is administered at a dosage of in the range of from about 0.1 mg to about 500 mg, from about 1 mg to about 500 mg, from about 10 mg to about 500 mg, from about 50 mg to about 500 mg, from about 100 mg to about 400 mg, from about 200 mg to about 400 mg, from about 250 mg to about 350 mg, or about 300 mg. In one embodiment, the composition comprises the PI3K delta inhibitor (e.g., Idelalisib), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta inhibitor (e.g., Idelalisib), or a pharmaceutically acceptable form thereof, is administered at a dosage of less than about 500 mg, less than about 400 mg, less than about 350 mg, less than about 300 mg, less than about 250 mg, less than about 200 mg, less than about 150 mg, less than about 100 mg, less than about 75 mg, less than about 50 mg, less than about 30 mg, less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In one embodiment, the composition comprises the PI3K delta/gamma inhibitor (e.g., Compound 1), or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises the PI3K delta/gamma inhibitor (e.g., Compound 1), or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises the PI3K delta/gamma inhibitor which is Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment, the PI3K delta/gamma dual inhibitor (e.g., Compound 1) is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr.

In one embodiment, the PI3K delta/gamma dual inhibitor (e.g., Compound 1) is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, the PI3K delta/gamma dual inhibitor (e.g., Compound 1) is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL.

In one embodiment, the PI3K delta/gamma dual inhibitor (e.g., Compound 1) is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the composition comprises the PI3K delta/gamma dual inhibitor (e.g., Compound 1), or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises the PI3K delta/gamma dual inhibitor (e.g., Compound 1), or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg.

In one embodiment, the PI3K delta/gamma dual inhibitor (e.g., Compound 1), or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, the PI3K delta/gamma dual inhibitor (e.g., Compound 1), or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily.

In one embodiment, the composition comprises Compound 1, or a pharmaceutically acceptable form thereof, at an amount sufficient to deliver a blood plasma concentration profile with an AUC (area under curve) of from about 1 ng/mL*h to about 1 mg/mL*h, from about 10 ng/mL*h to about 100 μg/mL*h, from about 100 ng/mL*h to about 10 μg/mL*h, from about 1 μg/mL*h to about 10 μg/mL*h. In one embodiment the composition comprises Compound 1, or a pharmaceutically acceptable form thereof, at an amount sufficient to deliver a blood plasma concentration profile with an AUC (area under curve) of from about 0.1 μg/mL*h to about 10 μg/mL*h, from about 0.2 μg/mL*h to about 9 μg/mL*h, from about 0.3 μg/mL*h to about 8 μg/mL*h, from about 0.4 μg/mL*h to about 7 μg/mL*h, from about 0.5 μg/mL*h to about 6 μg/mL*h, from about 0.6 μg/mL*h to about 5 μg/mL*h, from about 0.7 μg/mL*h to about 4 μg/mL*h, from about 0.8 μg/mL*h to about 3 μg/mL*h, from about 0.9 μg/mL*h to about 2 μg/mL*h, or from about 0.9 μg/mL*h to about 1 μg/mL*h. In one embodiment the composition comprises Compound 1, or a pharmaceutically acceptable form thereof, at an amount sufficient to deliver a blood plasma concentration profile with an AUC (area under curve) of from about 1 μg/mL*h to about 10 μg/mL*h, from about 5 μg/mL*h to about 9 μg/mL*h, or from about 6 μg/mL*h to about 8 μg/mL*h.

In one embodiment Compound 1 is administered at an amount to reach an area under the plasma concentration-time curve at steady-state (AUCss) at about 5000 ng/mL*hr to about 10000 ng/mL*hr, about 5000 ng/mL*hr to about 9000 ng/mL*hr, about 6000 ng/mL*hr to about 9000 ng/mL*hr, about 7000 ng/mL*hr to about 9000 ng/mL*hr, about 8000 ng/mL*hr to about 9000 ng/mL*hr, or about 8787 ng/mL*hr.

In one embodiment, Compound 1 is administered at an amount to reach an area under the plasma concentration-time curve at steady-state (AUCss) at less than about 10000 ng/mL*hr, less than about 9500 ng/mL*hr, less than about 9000 ng/mL*hr, less than about 8500 ng/mL*hr, less than about 8000 ng/mL*hr, less than about 7000 ng/mL*hr, less than about 6000 ng/mL*hr, less than about 5000 ng/mL*hr, less than about 4000 ng/mL*hr, less than about 3000 ng/mL*hr, less than about 2000 ng/mL*hr, less than about 1000 ng/mL*hr, less than about 500 ng/mL*hr, less than about 100 ng/mL*hr, less than about 10 ng/mL*hr, or less than about 1 ng/mL*hr.

In one embodiment, Compound 1 is administered at an amount to reach maximum plasma concentration at steady state (Cmaxss) at about 1000 ng/mL to about 5000 ng/mL, about 1000 ng/mL to about 4000 ng/mL, about 1000 ng/mL to about 3000 ng/mL, about 1000 ng/mL to about 2500 ng/mL, about 1400 ng/mL to about 2000 ng/mL, about 1400 ng/mL to about 1500 ng/mL, or about 1487 ng/mL.

In one embodiment, Compound 1 is administered at an amount to reach maximum plasma concentration at steady state (Cmaxss) at less than about 5000 ng/mL, less than about 4000 ng/mL, less than about 3000 ng/mL, less than about 2000 ng/mL, less than about 1500 ng/mL, less than about 1000 ng/mL, less than about 500 ng/mL, less than about 100 ng/mL, less than about 50 ng/mL, less than about 25 ng/mL, less than about 10 ng/mL, or less than about 1 ng/mL.

In one embodiment, the composition comprises Compound 1, or a pharmaceutically acceptable form thereof, at an amount in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg.

In one embodiment, the composition comprises Compound 1, or a pharmaceutically acceptable form thereof, at an amount of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg. In one embodiment, the composition comprises Compound 1, or a pharmaceutically acceptable form thereof, at an amount of about 50 mg, about 37.5 mg, about 25 mg, about 20 mg, about 15 mg, about 10 mg, about 5 mg, or about 1 mg.

In one embodiment, Compound 1, or a pharmaceutically acceptable form thereof, is administered at a dosage of in the range of from about 0.1 mg to about 75 mg, from about 1 mg to about 75 mg, from about 5 mg to about 75 mg, from about 5 mg to about 60 mg, from about 5 mg to about 50 mg, from about 5 mg to about 30 mg, from about 5 mg to about 25 mg, from about 10 mg to about 25 mg, or from about 10 mg to about 20 mg daily.

In one embodiment, Compound 1, or a pharmaceutically acceptable form thereof, is administered at a dosage of less than about 25 mg, less than about 20 mg, less than about 19 mg, less than about 18 mg, less than about 17 mg, less than about 16 mg, less than about 16 mg, less than about 15 mg, less than about 14 mg, less than about 13 mg, less than about 12 mg, less than about 11 mg, or less than about 10 mg daily. In one embodiment, Compound 1, or a pharmaceutically acceptable form thereof, is administered at a dosage of about 50 mg, about 37.5 mg, about 25 mg, about 20 mg, about 15 mg, about 10 mg, about 5 mg, or about 1 mg daily.

Any of the compounds disclosed herein can be in the form of pharmaceutically acceptable salts, hydrates, solvates, chelates, non-covalent complexes, isomers, prodrugs, isotopically labeled derivatives, or mixtures thereof.

### 2.2 Combinations of PI3K Inhibitors and Checkpoint Modulators

In certain embodiments, provided herein are methods of treating, managing, or preventing a cancer in a subject comprising administering to the subject a therapeutically effective amount of a PI3K inhibitor, or a pharmaceutically acceptable form thereof, in combination with a checkpoint modulator, or a pharmaceutically acceptable form thereof.

In certain embodiments, provided herein are pharmaceutical compositions comprising a therapeutically effective amount of a PI3K inhibitor, or a pharmaceutically acceptable form thereof, and a checkpoint modulator, or a pharmaceutically acceptable form thereof.

As used herein, the term “immune checkpoint modulator” or “checkpoint modulator” refers to molecules that totally or partially interfere with or modulate one or more checkpoint molecules. In one embodiment, the checkpoint modulator is a “checkpoint inhibitor”, which refers to a molecule that inhibits, decreases or interferes with the activity of an inhibitory checkpoint molecule. Without being bound by a particular theory, an inhibitory checkpoint molecule down-regulates immune responses (e.g., T-cell activation) by delivery of a negative signal to T-cells following their engagement by ligands or counter-receptors. In another embodiment, the checkpoint modulator is an activator of a costimulatory molecule.

In certain embodiments, checkpoint inhibitors for use with the methods and compositions provided herein can inhibit the activity of an inhibitory checkpoint molecule directly, or decrease the expression of an inhibitory checkpoint molecule, or interfere with the interaction of an inhibitory checkpoint molecule and a binding partner (e.g., a ligand). The checkpoint modulators for use with the methods and compositions provided herein include, but are not limited to, a protein, a polypeptide, a peptide, an antisense oligonucleotide, an antibody, an antibody fragment, or an RNA molecule (e.g., an inhibitory RNA molecule that targets the expression of an inhibitory checkpoint molecule).

In certain embodiments, the inhibitory checkpoint molecule is selected from the group consisting of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092.

In one embodiment, the immune checkpoint modulator is an inhibitor of an inhibitory checkpoint molecule, for instance, an inhibitor of PD-1, PD-L1, PD-L2, CTLA-4, TIM3, LAG3, VISTA, BTLA, TIGIT, LAIR1, CD160, 2B4 and/or TGFR beta. For instance, the inhibitor of an inhibitory checkpoint molecule may inhibit PD-1, PD-L1, LAG-3, TIM-3 or CTLA-4, or any combination thereof.

Inhibition of an inhibitory molecule can be performed at the DNA, RNA or protein level. For example, an inhibitory nucleic acid (e.g., a dsRNA, siRNA or shRNA), can be used to inhibit expression of an inhibitory molecule. In other embodiments, the inhibitor of an inhibitory signal is, a polypeptide, e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

The antibody molecule may be, e.g., a full antibody or fragment thereof (e.g., a Fab, F(ab′)₂, Fv, or a single chain Fv fragment (scFv)). The antibody molecule may be, e.g., in the form of a bispecific antibody molecule. In one embodiment, the bispecific antibody molecule has a first binding specificity to PD-1 or PD-L1 and a second binding specifity, e.g., a second binding specificity to TIM-3, LAG-3, or PD-L2.

In certain embodiments, the immune checkpoint modulator is an inhibitor of PD-1, e.g., human PD-1. In another embodiment, the immune checkpoint modulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immune checkpoint modulators, e.g., in combination with an inhibitor of LAG-3, TIM-3 or CTLA-4. In some embodiments, the inhibitor of PD-1 or PD-L1, e.g., the anti-PD-1 or anti-PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. In yet another embodiment, provided herein are other combinations of immune checkpoint modulators with a PD-1 inhibitor (e.g., one or more of PD-L2, CTLA-4, TIM3, LAG3, VISTA, BTLA, TIGIT, LAIR1, CD160, 2B4 and/or TGFR). In one embodiment, the PI3K inhibitor molecules disclosed herein are used in the aforesaid combinations of inhibitors of checkpoint molecule.

In one embodiment, the checkpoint modulator is a PD-1/PD-L1 inhibitor. Examples of PD-1/PD-L1 inhibitors include, but are not limited to, those described in U.S. Pat. Nos. 7,488,802, 7,943,743, 8,008,449, 8,168,757, 8,217,149, 8,609,089, US 2010/028330, US 2012/0114649, WO 2003/042402, WO 2008/156712, WO 2010/089411, WO 2010/036959, WO 2011/066342, WO 2011/159877, WO 2011/082400, and WO 2011/161699, all of which are incorporated herein in their entireties.

In one embodiment, the checkpoint modulator is a PD-1 inhibitor. In one embodiment, the checkpoint modulator is an anti-PD-1 antibody.

In some embodiments, the anti-PD-1 antibody is Nivolumab. Alternative names for Nivolumab include MDX-1106, MDX-1106-04, ONO-4538, or BMS-936558, and has a CAS Registry Number: 946414-94-4. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO 2006/121168.

In other embodiments, the anti-PD-1 antibody is Pembrolizumab. Pembrolizumab (Trade name KEYTRUDA formerly Lambrolizumab,—also known as Merck 3745, MK-3475 or SCH-900475) is a humanized IgG4 monoclonal antibody that binds to PD-1. Pembrolizumab is disclosed, e.g., in Hamid, O. et al. (2013) New England Journal ofMedicine 369 (2): 134-44, WO 2009/114335, and U.S. Pat. No. 8,354,509.

In some embodiments, the anti-PD-1 antibody is Pidilizumab. Pidilizumab (CT-011; Cure Tech) is a humanized IgG1k monoclonal antibody that binds to PD-1. Pidilizumab and other humanized anti-PD-1 monoclonal antibodies are disclosed in WO 2009/101611. Other anti-PD1 antibodies are disclosed in U.S. Pat. No. 8,609,089, US 2010/028330, and/or US 2012/0114649.

In some embodiments, the anti-PD-1 antibody is AMP-514 (Amplimmune).

In some embodiments, the anti-PD-1 antibody is AMP-224, a fusion protein.

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)).

In one embodiment, the checkpoint modulator is a PD-L1 inhibitor. In one embodiment, the checkpoint modulator is an anti-PD-L1 antibody.

In one embodiment, the anti-PD-L1 antibody is MDX-1105. MDX-1105, also known as BMS-936559, is an anti-PD-L1 antibody, as described in WO 2007/005874.

In one embodiment, the anti-PD-L1 antibody is YW243.55.S70. The YW243.55.S70 antibody is an anti-PD-L1 antibody, as described in WO 2010/077634. Heavy and light chain variable region sequences of YW243.55.S70 are also described in WO 2010/077634.

In one embodiment, the anti-PD-L1 antibody is MDPL3280A (Genentech/Roche). MDPL3280A is a human Fc optimized IgG1 monoclonal antibody that binds to PD-L1. MDPL3280A and other human monoclonal antibodies to PD-L1 are described in U.S. Pat. No. 7,943,743 and US 2012/0039906.

In one embodiment, the anti-PD-L1 antibody is MSB0010718C. MSB0010718C (also referred to as A09-246-2; Merck Serono) is a monoclonal antibody that binds to PD-L1. Other humanized anti-PD-L1 antibodies are disclosed in WO 2013/079174.

In one embodiment, the anti-PD-L1 antibody is durvalumab (also known as MEDI-4736).

In one embodiment, the checkpoint modulator is a PD-L2 inhibitor. In one embodiment, the checkpoint modulator is an anti-PD-L2 antibody. In one embodiment, the anti-PD-L2 antibody is rHIgM12B7A.

In one embodiment, the checkpoint modulator is a lymphocyte activation gene-3 (LAG-3) inhibitor. In one embodiment, the checkpoint modulator is an anti-LAG-3 antibody. In one embodiment, the anti-LAG-3 antibody is BMS-986016. BMS-986016 (Bristol-Myers Squibb) is a monoclonal antibody that binds to LAG-3. BMS-986016 and other humanized anti-LAG-3 antibodies are described in US 2011/0150892, WO 2010/019570, and WO 2014/008218. In another embodiment, the anti-LAG-3 antibody is IMP321, a soluble Ig fusion protein (Brignone et al., J. Immunol., 2007, 179, 4202-4211).

In one embodiment, the checkpoint modulator is a soluble ligand (e.g., a CTLA-4-Ig), or an antibody or antibody fragment that binds to CTLA-4. In one embodiment, the checkpoint modulator is a CTLA-4 inhibitor. In one embodiment, the checkpoint modulator is an anti-CTLA-4 antibody. Examples of anti-CTLA-4 antibodies include, but are not limited to, those described in U.S. Pat. Nos. 5,811,097, 5,811,097, 5,855,887, 6,051,227, 6,207,157, 6,682,736, 6,984,720, and 7,605,238, all of which are incorporated herein in their entireties.

In one embodiment, the anti-CTLA-4 antibody is Tremelimumab (IgG2 monoclonal antibody available from Pfizer, formerly known as ticilimumab, CP-675,206).

In one embodiment, the anti-CTLA-4 antibody is Ipilimumab (also known as MDX-010, Yervoy, CAS No. 477202-00-9). Ipilimumab is a fully human monoclonal IgG antibody that binds to CTLA-4.

In one embodiment, the checkpoint modulator is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint modulator is a TIM3 inhibitor (Fourcade et al., J. Exp. Med., 2010, 207, 2175-86; Sakuishi et al., J. Exp. Med., 2010, 207, 2187-94).

In one embodiment, the checkpoint modulator is an IDO (indoleamine 2,3-dioxygenase) and/or TDO (tryptophan 2,3-dioxygenase) inhibitor. In one embodiment, the checkpoint modulator is an IDO inhibitor. In one embodiment, the IDO inhibitor is indoximod, NLG919, INCB024360, F001287, norharmane, rosmarinic acid, or alpha-methyl-tryptophan. In one embodiment, the IDO inhibitor is INCB024360. In another embodiment, the IDO inhibitor is indoximod. Although IDO inhibitors act within the TME, they do not specifically target MDSCs. The overexpression of IDO by dendritic cells creates an immunosuppressive tumor microenvironment.

In one embodiment, the checkpoint modulator is an activator of a costimulatory molecule. In one embodiment, the checkpoint modulator is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In one embodiment, the checkpoint modulator is an agonist of OX40. In one embodiment, the checkpoint modulator is an anti-OX40 antibody. In one embodiment, the anti-OX40 antibody is MEDI6469.

In one embodiment, the checkpoint modulator is an agonist associated with a positive signal that includes a costimulatory domain of CD28, CD27, ICOS and GITR.

In one embodiment, the checkpoint modulator is an agonist of GITR. In one embodiment, the checkpoint modulator is an anti-GITR antibody. Exemplary GITR agonists include, e.g., GITR fusion proteins and anti-GITR antibodies (e.g., bivalent anti-GITR antibodies), such as, a GITR fusion protein described in U.S. Pat. No. 6,111,090, EP 090505 B1, U.S. Pat. No. 8,586,023, WO 2010/003118 and WO 2011/090754, or an anti-GITR antibody described, e.g., in U.S. Pat. No. 7,025,962, EP 1947183 B1, U.S. Pat. Nos. 7,812,135, 8,388,967, 8,591,886, EP 1866339, WO 2011/028683, WO 2013/039954, WO 2005/007190, WO 2007/133822, WO 2005/055808, WO 99/40196, WO 2001/03720, WO 99/20758, WO 2006/083289, WO 2005/115451, U.S. Pat. No. 7,618,632, and WO 2011/051726. In one embodiment, the anti-GITR antibody is TRX518.

In one embodiment, the checkpoint modulator is a CD137 agonist. In one embodiment, the checkpoint modulator is an anti-CD137 antibody. In one embodiment, the anti-CD137 antibody is urelumab. In another embodiment, the anti-CD137 antibody is PF-05082566.

In one embodiment, the checkpoint modulator is a CD40 agonist. In one embodiment, the checkpoint modulator is an anti-CD40 antibody. In one embodiment, the anti-CD40 antibody is CF-870,893.

In some embodiments, the checkpoint modulator is a costimulatory ligand. In some embodiments, the costimulatory ligand is OX40L, 41BBL, CD153, ICOSL, CD40L, or GMCSF.

In some embodiments, the checkpoint modulator is a MCSF/CSF-1R inhibitor. An anti-CSF-1R can deplete TAMs, resulting in tumor growth inhibition. Cancer Cell 25, 1-14, Jun. 16, 2014. In some embodiments, the CSF-1R inhibitor is BLZ945, GW2850, R05509554, or PLX3397. In some embodiments, the CSF-1R inhibitor is BLZ945 or GW2850. In some embodiments, the CSF-1R inhibitor is PLX3397.

In some embodiments, the checkpoint modulator is a CXCR4/CXCL12 inhibitor. In some embodiments, the CXCR4/CXCL12 inhibitor is AMD3100, AMD11070, AMD12118, AMD11814, or AMD13073. In some embodiments, the CXCR4/CXCL12 inhibitor is AMD3100.

In some embodiments, the checkpoint modulator is a CCL2 and/or CCR2 antagonist. In some embodiments, the antagonist of CCL2 and/or CCR2 is an anti-CCL2 or CCR2 antibody. CCL2 is a chemokine and CCR2 is a chemokine receptor. CCL2 and CCR2, according to non-limiting theory, play a role in MDSC migration.

In one embodiment, provided herein is a method of treating, managing, or preventing a cancer in a subject comprising administering to the subject a therapeutically effective amount of a PI3K inhibitor, or a pharmaceutically acceptable form thereof, in combination with a checkpoint modulator, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a checkpoint modulator, or a pharmaceutically acceptable form thereof, wherein the PI3K inhibitor is a PI3K delta inhibitor.

In one embodiment of the compositions and methods provided herein, the PI3K inhibitor is a PI3K delta inhibitor, and the checkpoint modulator is an anti-PD-1 antibody, an anti-PD-L1 antibody, an anti-PD-L2 antibody, or an anti-CTLA-4 antibody, or a combination thereof. In one embodiment, the anti-PD-1 antibody is Nivolumab, Pembrolizumab, Pidilizumab, AMP-514, or AMP-224, or a combination thereof. In one embodiment, the anti-PD-L1 antibody is MDX-1105, YW243.55.S70, MDPL3280A, MSB0010718C, or durvalumab, or a combination thereof. In one embodiment, the an anti-CTLA-4 antibody is Tremelimumab or Ipilimumab, or a combination thereof.

In one embodiment, the PI3K inhibitor is a PI3K delta inhibitor, and the checkpoint modulator is Nivolumab.

In one embodiment, the PI3K inhibitor is a PI3K delta inhibitor, and the checkpoint modulator is Pembrolizumab.

In one embodiment, the PI3K inhibitor is a PI3K delta inhibitor, and the checkpoint modulator is Pidilizumab.

In one embodiment, the PI3K inhibitor is a PI3K delta inhibitor, and the checkpoint modulator is MDX-1105.

In one embodiment, the PI3K inhibitor is a PI3K delta inhibitor, and the checkpoint modulator is MDPL3280A.

In one embodiment, the PI3K inhibitor is a PI3K delta inhibitor, and the checkpoint modulator is durvalumab.

In one embodiment, the PI3K inhibitor is a PI3K delta inhibitor, and the checkpoint modulator is Tremelimumab.

In one embodiment, the PI3K inhibitor is a PI3K delta inhibitor, and the checkpoint modulator is Ipilimumab.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the checkpoint modulator, or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the checkpoint modulator, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and the checkpoint modulator is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a checkpoint modulator, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a checkpoint modulator, or a pharmaceutically acceptable form thereof, wherein the PI3K inhibitor is Idelalisib.

In one embodiment of the compositions and methods provided herein, the PI3K inhibitor is Idelalisib, and the checkpoint modulator is an anti-PD-1 antibody, an anti-PD-L1 antibody, an anti-PD-L2 antibody, or an anti-CTLA-4 antibody, or a combination thereof. In one embodiment, the anti-PD-1 antibody is Nivolumab, Pembrolizumab, Pidilizumab, AMP-514, or AMP-224, or a combination thereof. In one embodiment, the anti-PD-L1 antibody is MDX-1105, YW243.55.S70, MDPL3280A, MSB0010718C, or durvalumab, or a combination thereof. In one embodiment, the an anti-CTLA-4 antibody is Tremelimumab or Ipilimumab, or a combination thereof.

In one embodiment, the PI3K inhibitor is Idelalisib, and the checkpoint modulator is Nivolumab.

In one embodiment, the PI3K inhibitor is Idelalisib, and the checkpoint modulator is Pembrolizumab.

In one embodiment, the PI3K inhibitor is Idelalisib, and the checkpoint modulator is Pidilizumab.

In one embodiment, the PI3K inhibitor is Idelalisib, and the checkpoint modulator is MDX-1105.

In one embodiment, the PI3K inhibitor is Idelalisib, and the checkpoint modulator is MDPL3280A.

In one embodiment, the PI3K inhibitor is Idelalisib, and the checkpoint modulator is durvalumab.

In one embodiment, the PI3K inhibitor is Idelalisib, and the checkpoint modulator is Tremelimumab.

In one embodiment, the PI3K inhibitor is Idelalisib, and the checkpoint modulator is Ipilimumab.

In one embodiment of the methods described herein, Idelalisib, or a pharmaceutically acceptable form thereof, is administered at a dosage of in the range of from about 0.01 mg to about 75 mg daily and the checkpoint modulator, or a pharmaceutically acceptable form thereof, is administered at a dosage of in the range of from about 0.01 mg to about 1100 mg daily.

In one embodiment of the compositions and methods described herein, the molar ratio of Idelalisib, or a pharmaceutically acceptable form thereof, to the checkpoint modulator, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the checkpoint modulator is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a checkpoint modulator, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a checkpoint modulator, or a pharmaceutically acceptable form thereof, wherein the PI3K inhibitor is a PI3K delta/gamma dual inhibitor.

In one embodiment of the compositions and methods provided herein, the PI3K inhibitor is a PI3K delta/gamma dual inhibitor, and the checkpoint modulator is an anti-PD-1 antibody, an anti-PD-L1 antibody, an anti-PD-L2 antibody, or an anti-CTLA-4 antibody, or a combination thereof. In one embodiment, the anti-PD-1 antibody is Nivolumab, Pembrolizumab, Pidilizumab, AMP-514, or AMP-224, or a combination thereof. In one embodiment, the anti-PD-L1 antibody is MDX-1105, YW243.55.S70, MDPL3280A, MSB0010718C, or durvalumab, or a combination thereof. In one embodiment, the an anti-CTLA-4 antibody is Tremelimumab or Ipilimumab, or a combination thereof.

In one embodiment, the PI3K inhibitor is a PI3K delta/gamma dual inhibitor, and the checkpoint modulator is Nivolumab.

In one embodiment, the PI3K inhibitor is a PI3K delta/gamma dual inhibitor, and the checkpoint modulator is Pembrolizumab.

In one embodiment, the PI3K inhibitor is a PI3K delta/gamma dual inhibitor, and the checkpoint modulator is Pidilizumab.

In one embodiment, the PI3K inhibitor is a PI3K delta/gamma dual inhibitor, and the checkpoint modulator is MDX-1105.

In one embodiment, the PI3K inhibitor is a PI3K delta/gamma dual inhibitor, and the checkpoint modulator is MDPL3280A.

In one embodiment, the PI3K inhibitor is a PI3K delta/gamma dual inhibitor, and the checkpoint modulator is durvalumab.

In one embodiment, the PI3K inhibitor is a PI3K delta/gamma dual inhibitor, and the checkpoint modulator is Tremelimumab.

In one embodiment, the PI3K inhibitor is a PI3K delta/gamma dual inhibitor, and the checkpoint modulator is Ipilimumab.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the checkpoint modulator, or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the checkpoint modulator, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the checkpoint modulator is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a checkpoint modulator, or a pharmaceutically acceptable form thereof, wherein the PI3K inhibitor is Compound 1:

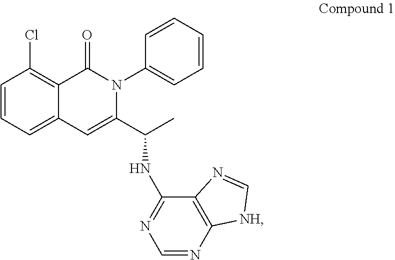 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a checkpoint modulator, or a pharmaceutically acceptable form thereof, wherein the PI3K inhibitor is Compound 1:

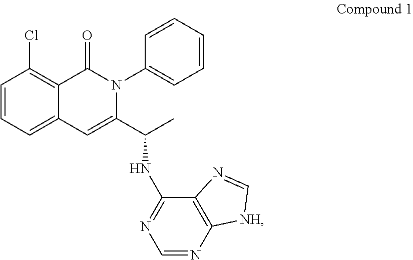 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the compositions and methods provided herein, the PI3K inhibitor is Compound 1, and the checkpoint modulator is an anti-PD-1 antibody, an anti-PD-L1 antibody, an anti-PD-L2 antibody, or an anti-CTLA-4 antibody, or a combination thereof. In one embodiment, the anti-PD-1 antibody is Nivolumab, Pembrolizumab, Pidilizumab, AMP-514, or AMP-224, or a combination thereof. In one embodiment, the anti-PD-L1 antibody is MDX-1105, YW243.55.S70, MDPL3280A, MSB0010718C, or durvalumab, or a combination thereof. In one embodiment, the an anti-CTLA-4 antibody is Tremelimumab or Ipilimumab, or a combination thereof.

In one embodiment, the PI3K inhibitor is Compound 1, and the checkpoint modulator is Nivolumab.

In one embodiment, the PI3K inhibitor is Compound 1, and the checkpoint modulator is Pembrolizumab.

In one embodiment, the PI3K inhibitor is Compound 1, and the checkpoint modulator is Pidilizumab.

In one embodiment, the PI3K inhibitor is Compound 1, and the checkpoint modulator is MDX-1105.

In one embodiment, the PI3K inhibitor is Compound 1, and the checkpoint modulator is MDPL3280A.

In one embodiment, the PI3K inhibitor is Compound 1, and the checkpoint modulator is durvalumab.

In one embodiment, the PI3K inhibitor is Compound 1, and the checkpoint modulator is Tremelimumab.

In one embodiment, the PI3K inhibitor is Compound 1, and the checkpoint modulator is Ipilimumab.

In one embodiment of the methods described herein, Compound 1, or a pharmaceutically acceptable form thereof, is administered at a dosage of in the range of from about 0.01 mg to about 75 mg daily and the checkpoint modulator, or a pharmaceutically acceptable form thereof, is administered at a dosage of in the range of from about 0.01 mg to about 1100 mg daily.

In one embodiment of the compositions and methods described herein, the molar ratio of Compound 1, or a pharmaceutically acceptable form thereof, to the checkpoint modulator, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and the checkpoint modulator is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the PI3K inhibitor is Compound 1, the checkpoint modulator is nivolumab, and the cancer is T-cell lymphoma. In one embodiment, the T-cell lymphoma is peripheral T cell lymphomas (PTCL). In another embodiment, the T-cell lymphoma is cutaneous T-cell lymphoma (CTCL).

In one embodiment, the PI3K inhibitor is Compound 1, the checkpoint modulator is nivolumab, and the cancer is DLBCL.

In one embodiment, the PI3K inhibitor is Compound 1, the checkpoint modulator is nivolumab, and the cancer is follicular lymphoma.

In one embodiment, the PI3K inhibitor is Compound 1, the checkpoint modulator is pembrolizumab, and the cancer is T-cell lymphoma. In one embodiment, the T-cell lymphoma is peripheral T cell lymphomas (PTCL). In another embodiment, the T-cell lymphoma is cutaneous T-cell lymphoma (CTCL).

In one embodiment, the PI3K inhibitor is Compound 1, the checkpoint modulator is pembrolizumab, and the cancer is DLBCL.

In one embodiment, the PI3K inhibitor is Compound 1, the checkpoint modulator is pembrolizumab, and the cancer is follicular lymphoma.

In one embodiment of the methods provided herein, the checkpoint modulator is nivolumab, and it is administered intravenously. In one embodiment, nivolumab is administered at a dose of from about 0.1 mg/kg to about 5 mg/kg, from about 0.1 mg/kg to about 3 mg/kg, from about 0.1 mg/kg to about 2 mg/kg, from about 0.1 mg/kg to about 1 mg/kg, or from about 0.1 mg/kg to about 0.5 mg/kg every 2 weeks. In one embodiment, nivolumab is administered at a dose of about 3 mg/kg, about 2.5 mg/kg, about 2 mg/kg, about 1.5 mg/kg, about 1 mg/kg, or about 0.5 mg/kg every 2 weeks. In one embodiment, the frequency of the administration of nivolumab is reduced to once every 3 weeks or once every 4 weeks. In one embodiment, a dose of nivolumab is administered intravenously over about 60 minutes. In one embodiment, a dose of nivolumab is administered intravenously over about 30 minutes.

In one embodiment of the methods provided herein, the checkpoint modulator is pembrolizumab, and it is administered intravenously. In one embodiment, pembrolizumab is administered at a dose of from about 0.1 mg/kg to about 5 mg/kg, from about 0.1 mg/kg to about 3 mg/kg, from about 0.1 mg/kg to about 2 mg/kg, from about 0.1 mg/kg to about 1 mg/kg, or from about 0.1 mg/kg to about 0.5 mg/kg every 3 weeks. In one embodiment, pembrolizumab is administered at a dose of about 3 mg/kg, about 2.5 mg/kg, about 2 mg/kg, about 1.5 mg/kg, about 1 mg/kg, or about 0.5 mg/kg every 3 weeks. In one embodiment, the frequency of the administration of pembrolizumab is reduced to once every 4 weeks, once every 5 weeks, or once every 6 weeks. In one embodiment, a dose of pembrolizumab is administered intravenously over about 30 minutes. In one embodiment, a dose of pembrolizumab is administered intravenously over about 15 minutes.

In one embodiment, the checkpoint modulator, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the checkpoint modulator, or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the checkpoint modulator, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In certain embodiments, the PI3K inhibitor (e.g., Compound 1), or a pharmaceutically acceptable form thereof, and the checkpoint modulator are administered via a same route, e.g., both are administered orally. In other embodiments, the PI3K inhibitor (e.g., Compound 1), or a pharmaceutically acceptable form thereof, and the checkpoint modulator are administered via different routes, e.g., one is administered orally and the other is administered intravenously. In one embodiment, Compound 1 is administered orally and the checkpoint modulator is administered intravenously.

In certain embodiments, the PI3K inhibitor (e.g., Compound 1), or a pharmaceutically acceptable form thereof, and the checkpoint modulator, or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and a checkpoint modulator.

### 2.3 Combinations of PI3K Inhibitors and XPO1 Inhibitors

In certain embodiments, provided herein are methods of treating, managing, or preventing a cancer in a subject comprising administering to the subject a therapeutically effective amount of a PI3K inhibitor, or a pharmaceutically acceptable form thereof, in combination with an XPO1 inhibitor, or a pharmaceutically acceptable form thereof.

In certain embodiments, provided herein are pharmaceutical compositions comprising a therapeutically effective amount of a PI3K inhibitor, or a pharmaceutically acceptable form thereof, and an XPO1 inhibitor, or a pharmaceutically acceptable form thereof.

Exportin 1 (XPO1), also known as chromosomal maintenance 1 (CRM1), is an eukaryotic protein that mediates the nuclear export of proteins, rRNA, snRNA, and some mRNA. In certain embodiments, the XPO1 inhibitors for use in the methods and compositions provided herein include, but are not limited to, selinexor, KPT-251, KPT-276, and SL-801.

In one embodiment, the XPO1 inhibitor is selinexor. Selinexor, also known as KPT-330, has a chemical name of (Z)-3-(3-(3,5-bis(trifluoromethyl)phenyl)-1H-1,2,4-triazol-1-yl)-N′-(pyrazin-2-yl)acrylohydrazide, and is of the structure:

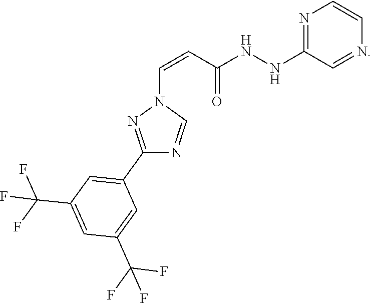 [SMILES: O=C(/C=C\n1cnc(-c2cc(C(F)(F)F)cc(C(F)(F)F)c2)n1)NNc1cnccn1]

In one embodiment, the XPO1 inhibitor is SL-801.

In one embodiment, provided herein is a method of treating, managing, or preventing a cancer in a subject comprising administering to the subject a therapeutically effective amount of a PI3K inhibitor, or a pharmaceutically acceptable form thereof, in combination with a XPO1 inhibitor, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a XPO1 inhibitor, or a pharmaceutically acceptable form thereof, wherein the PI3K inhibitor is a PI3K delta inhibitor.

In one embodiment of the compositions and methods provided herein, the PI3K inhibitor is a PI3K delta inhibitor, and the XPO1 inhibitor is selinexor, KPT-251, KPT-276, or SL-801, or a combination thereof.

In one embodiment, the PI3K inhibitor is a PI3K delta inhibitor, and the XPO1 inhibitor is selinexor.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the XPO1 inhibitor (e.g., selinexor), or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the XPO1 inhibitor (e.g., selinexor), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the XPO1 inhibitor (e.g., selinexor) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a XPO1 inhibitor, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a XPO1 inhibitor, or a pharmaceutically acceptable form thereof, wherein the PI3K inhibitor is Idelalisib.

In one embodiment of the compositions and methods provided herein, the PI3K inhibitor is Idelalisib, and the XPO1 inhibitor is selinexor, KPT-251, KPT-276, or SL-801, or a combination thereof.

In one embodiment, the PI3K inhibitor is Idelalisib, and the XPO1 inhibitor is selinexor.

In one embodiment of the methods described herein, Idelalisib, or a pharmaceutically acceptable form thereof, is administered at a dosage of in the range of from about 0.01 mg to about 75 mg daily and the XPO1 inhibitor (e.g., selinexor), or a pharmaceutically acceptable form thereof, is administered at a dosage of in the range of from about 0.01 mg to about 1100 mg daily.

In one embodiment of the compositions and methods described herein, Idelalisib, or a pharmaceutically acceptable form thereof, to the XPO1 inhibitor (e.g., selinexor), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the XPO1 inhibitor (e.g., selinexor) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a XPO1 inhibitor, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a XPO1 inhibitor, or a pharmaceutically acceptable form thereof, wherein the PI3K inhibitor is a PI3K delta/gamma dual inhibitor.

In one embodiment of the compositions and methods provided herein, the PI3K inhibitor is a PI3K delta/gamma dual inhibitor, and the XPO1 inhibitor is selinexor, KPT-251, KPT-276, or SL-801, or a combination thereof.

In one embodiment, the PI3K inhibitor is a PI3K delta/gamma dual inhibitor, and the XPO1 inhibitor is selinexor.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the XPO1 inhibitor (e.g., selinexor), or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the XPO1 inhibitor (e.g., selinexor), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the XPO1 inhibitor (e.g., selinexor) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a XPO1 inhibitor, or a pharmaceutically acceptable form thereof, wherein the PI3K inhibitor is Compound 1:

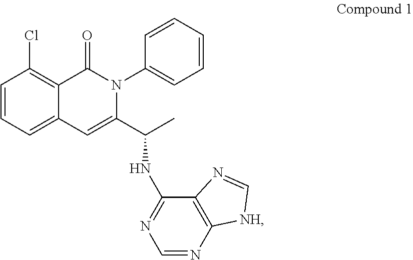 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a XPO1 inhibitor, or a pharmaceutically acceptable form thereof, wherein the PI3K inhibitor is Compound 1:

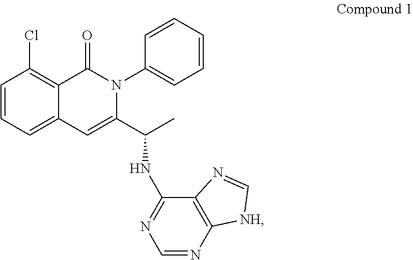 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the compositions and methods provided herein, the PI3K inhibitor is Compound 1, and the XPO1 inhibitor is selinexor, KPT-251, KPT-276, or SL-801, or a combination thereof.

In one embodiment, the PI3K inhibitor is Compound 1, and the XPO1 inhibitor is selinexor.

In one embodiment of the methods described herein, Compound 1, or a pharmaceutically acceptable form thereof, is administered at a dosage of in the range of from about 0.01 mg to about 75 mg daily and the XPO1 inhibitor (e.g., selinexor), or a pharmaceutically acceptable form thereof, is administered at a dosage of in the range of from about 0.01 mg to about 1100 mg daily.

In one embodiment of the compositions and methods described herein, Compound 1, or a pharmaceutically acceptable form thereof, to the XPO1 inhibitor (e.g., selinexor), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the XPO1 inhibitor (e.g., selinexor) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the PI3K inhibitor is Compound 1, the XPO1 inhibitor is selinexor, and the cancer is a hematologic malignancy. In one embodiment, the hematologic malignancy is leukemia. In one embodiment, the hematologic malignancy is lymphoma. In one embodiment, the hematologic malignancy is myeloma.

In one embodiment, the PI3K inhibitor is Compound 1, the XPO1 inhibitor is selinexor, and the cancer is acute myeloid leukemia. In one embodiment, the AML is relapsed or refractory. In one embodiment, the AML is untreated. In one embodiment, the AML is adult acute myeloid leukemia with 11q23 (MLL) abnormalities, adult acute myeloid leukemia with Del(5q), adult acute myeloid leukemia with Inv(16)(p13; q22), adult acute myeloid leukemia with t(15; 17)(q22; q12), adult acute myeloid leukemia with t(16; 16)(p13; q22), adult acute myeloid leukemia with t(8; 21)(q22; q22), recurrent adult acute myeloid leukemia, or secondary acute myeloid leukemia.

In one embodiment, the PI3K inhibitor is Compound 1, the XPO1 inhibitor is selinexor, and the cancer is multiple myeloma. In one embodiment, the multiple myeloma is relapsed or refractory.

In one embodiment, the PI3K inhibitor is Compound 1, the XPO1 inhibitor is selinexor, and the cancer is diffuse large B-cell lymphoma. In one embodiment, the DLBCL is relapsed or refractory.

In one embodiment, the PI3K inhibitor is Compound 1, the XPO1 inhibitor is selinexor, and the cancer is acute lymphoblastic leukemia (ALL). In on embodiment, the ALL is relapsed or refractory.

In one embodiment, the PI3K inhibitor is Compound 1, the XPO1 inhibitor is selinexor, and the cancer is mixed phenotype acute leukemia (MPAL).

In one embodiment, the PI3K inhibitor is Compound 1, the XPO1 inhibitor is selinexor, and the cancer is prolymphocytic leukemia.

In one embodiment, the PI3K inhibitor is Compound 1, the XPO1 inhibitor is selinexor, and the cancer is chronic lymphocytic leukemia (CLL). In one embodiment, the CLL is relapsed or refractory.

In one embodiment, the PI3K inhibitor is Compound 1, the XPO1 inhibitor is selinexor, and the cancer is chronic myeloid leukemia (CML). In one embodiment, the CML is relapsed or refractory.

In one embodiment, the PI3K inhibitor is Compound 1, the XPO1 inhibitor is selinexor, and the cancer is non-Hodgin lymphoma (NHL). In one embodiment, the NHL is relapsed or refractory.

In one embodiment, the PI3K inhibitor is Compound 1, the XPO1 inhibitor is selinexor, and the cancer is Hodgin lymphoma (HL). In one embodiment, the HL is relapsed or refractory.

In one embodiment, the PI3K inhibitor is Compound 1, the XPO1 inhibitor is selinexor, and the cancer is aggressive B-cell lymphoma.

In one embodiment, the PI3K inhibitor is Compound 1, the XPO1 inhibitor is selinexor, and the cancer is mantle cell lymphoma (MCL). In one embodiment, the MCL is relapsed or refractory.

In one embodiment, the PI3K inhibitor is Compound 1, the XPO1 inhibitor is selinexor, and the cancer is follicular lymphoma (FL). In one embodiment, the FL is relapsed or refractory.

In one embodiment, the PI3K inhibitor is Compound 1, the XPO1 inhibitor is selinexor, and the cancer is Waldenstrom macroglobulinemia. In one embodiment, the Waldenstrom macroglobulinemia is relapsed or refractory.

In one embodiment, the PI3K inhibitor is Compound 1, the XPO1 inhibitor is selinexor, and the cancer is myelodysplastic syndrome (MDS). In one embodiment, the MDS is de novo myelodysplastic syndrome or Secondary Myelodysplastic Syndrome.

In one embodiment, the PI3K inhibitor is Compound 1, the XPO1 inhibitor is selinexor, and the cancer is T-cell lymphoma. In one embodiment, the T-cell lymphoma is peripheral T cell lymphomas (PTCL). In another embodiment, the T-cell lymphoma is cutaneous T-cell lymphoma (CTCL).

In one embodiment, the PI3K inhibitor is Compound 1, the XPO1 inhibitor is selinexor, and the cancer is a solid tumor. In one embodiment, the solid tumor is a pediatric solid tumor.

In one embodiment, the PI3K inhibitor is Compound 1, the XPO1 inhibitor is selinexor, and the cancer is glioma. In one embodiment, the glioma is recurrent.

In one embodiment, the PI3K inhibitor is Compound 1, the XPO1 inhibitor is selinexor, and the cancer is ovarian carcinoma. In one embodiment, the PI3K inhibitor is Compound 1, the XPO1 inhibitor is selinexor, and the cancer is endometrial carcinoma. In one embodiment, the PI3K inhibitor is Compound 1, the XPO1 inhibitor is selinexor, and the cancer is cervical carcinoma.

In one embodiment, the PI3K inhibitor is Compound 1, the XPO1 inhibitor is selinexor, and the cancer is breast cancer. In one embodiment, the breast cancer is triple negative breast cancer (TNBC).

In one embodiment, the PI3K inhibitor is Compound 1, the XPO1 inhibitor is selinexor, and the cancer is pancreatic cancer. In one embodiment, the pancreatic cancer is acinar cell adenocarcinoma of the pancreas, duct cell adenocarcinoma of the pancreas, or stage IV pancreatic cancer.

In one embodiment, the PI3K inhibitor is Compound 1, the XPO1 inhibitor is selinexor, and the cancer is liposarcoma. In one embodiment, the cancer is dedifferentiated liposarcoma.

In one embodiment, the PI3K inhibitor is Compound 1, the XPO1 inhibitor is selinexor, and the cancer is melanoma. In one embodiment, the melanoma is recurrent.

In one embodiment, the PI3K inhibitor is Compound 1, the XPO1 inhibitor is selinexor, and the cancer is rectal cancer. In one embodiment, the rectal cancer is locally advanced.

In one embodiment, the PI3K inhibitor is Compound 1, the XPO1 inhibitor is selinexor, and the cancer is colorectal cancer.

In one embodiment, the PI3K inhibitor is Compound 1, the XPO1 inhibitor is selinexor, and the cancer is prostate cancer. In one embodiment, the prostate cancer is metastatic castration-resistant prostate cancer (mCRPC).

In one embodiment, the PI3K inhibitor is Compound 1, the XPO1 inhibitor is selinexor, and the cancer is esophageal cancer or gastric cancer.

In one embodiment, the PI3K inhibitor is Compound 1, the XPO1 inhibitor is selinexor, and the cancer is salivary gland cancer.

In one embodiment, the PI3K inhibitor is Compound 1, the XPO1 inhibitor is selinexor, and the cancer is liver cancer.

In one embodiment, the PI3K inhibitor is Compound 1, the XPO1 inhibitor is selinexor, and the cancer is lung cancer. In one embodiment, the lung cancer is small cell lung cancer. In one embodiment, the lung cancer is recurrent. In one embodiment, the lung cancer is recurrent squamous cell lung carcinoma or stage IV squamous cell lung carcinoma.

In one embodiment of the methods described herein, the XPO1 inhibitor (e.g., selinexor), or a pharmaceutically acceptable form thereof, is administered at a dosage of in the range of from about 30 mg to about 200 mg twice weekly, from about 45 mg to about 150 mg twice weekly, or from about 60 mg to about 100 mg twice weekly. In one embodiment, the XPO1 inhibitor (e.g., selinexor), or a pharmaceutically acceptable form thereof, is administered at a dosage of about 20 mg, about 30 mg, about 40 mg, about 50 mg, about 60 mg, about 70 mg, about 80 mg, about 90 mg, or about 100 mg twice weekly. In one embodiment, the dosage is about 60 mg twice weekly. In one embodiment, the dosage is about 80 mg twice weekly. In one embodiment, the dosage is about 100 mg twice weekly. In one embodiment, the administration is in a 28 day cycle.

In one embodiment of the methods described herein, the XPO1 inhibitor (e.g., selinexor), or a pharmaceutically acceptable form thereof, is administered at a dosage of in the range of from about 30 mg to about 200 mg once weekly, from about 45 mg to about 150 mg once weekly, or from about 60 mg to about 100 mg once weekly. In one embodiment, the XPO1 inhibitor (e.g., selinexor), or a pharmaceutically acceptable form thereof, is administered at a dosage of about 20 mg, about 30 mg, about 40 mg, about 50 mg, about 60 mg, about 70 mg, about 80 mg, about 90 mg, or about 100 mg once weekly. In one embodiment, the dosage is about 60 mg once weekly. In one embodiment, the dosage is about 80 mg once weekly. In one embodiment, the dosage is about 100 mg once weekly. In one embodiment, the administration is in a 28 day cycle.

In one embodiment, the XPO1 inhibitor (e.g., selinexor), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before Compound 1, or a pharmaceutically acceptable form thereof, is administered. In another embodiment, the XPO1 inhibitor (e.g., selinexor), or a pharmaceutically acceptable form thereof, is administered concurrently with Compound 1, or a pharmaceutically acceptable form thereof, in a single dosage form or separate dosage forms. In yet another embodiment, the XPO1 inhibitor (e.g., selinexor), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after Compound 1, or a pharmaceutically acceptable form thereof, is administered. In one embodiment, the XPO1 inhibitor is selinexor.

In certain embodiments, the PI3K inhibitor (e.g., Compound 1), or a pharmaceutically acceptable form thereof, and the XPO1 inhibitor (e.g., selinexor), or a pharmaceutically acceptable form thereof, are in a single dosage form. In other embodiments, the PI3K inhibitor (e.g., Compound 1), or a pharmaceutically acceptable form thereof, and the XPO1 inhibitor (e.g., selinexor), or a pharmaceutically acceptable form thereof, are in separate dosage forms.

In certain embodiments, the PI3K inhibitor (e.g., Compound 1), or a pharmaceutically acceptable form thereof, and the XPO1 inhibitor (e.g., selinexor), are administered via a same route, e.g., both are administered orally. In other embodiments, the PI3K inhibitor (e.g., Compound 1), or a pharmaceutically acceptable form thereof, and the XPO1 inhibitor (e.g., selinexor), are administered via different routes, e.g., one is administered orally and the other is administered intravenously.

In certain embodiments, the PI3K inhibitor (e.g., Compound 1), or a pharmaceutically acceptable form thereof, and the XPO1 inhibitor (e.g., selinexor), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and an XPO1 inhibitor (e.g., selinexor).

### 2.4 Combinations of PI3K Inhibitors and Anti-CD19 Antibodies

In certain embodiments, provided herein are methods of treating, managing, or preventing a cancer in a subject comprising administering to the subject a therapeutically effective amount of a PI3K inhibitor, or a pharmaceutically acceptable form thereof, in combination with an anti-CD19 antibody, or a pharmaceutically acceptable form thereof.

In certain embodiments, provided herein are pharmaceutical compositions comprising a therapeutically effective amount of a PI3K inhibitor, or a pharmaceutically acceptable form thereof, and an anti-CD19 antibody, or a pharmaceutically acceptable form thereof.

B-lymphocyte antigen CD19, also known as CD19 (Cluster of Differentiation 19), is a protein that in humans is encoded by the CD19 gene. It is found on the surface of B-cells, a type of white blood cell.

In one embodiment, the anti-CD19 antibody is blinatumomab. Blinatumomab is a recombinant, single-chain monoclonal antibody that possesses antigen-recognition sites for CD3 and CD19.

In one embodiment, provided herein is a method of treating, managing, or preventing a cancer in a subject comprising administering to the subject a therapeutically effective amount of a PI3K inhibitor, or a pharmaceutically acceptable form thereof, in combination with a anti-CD19 antibody, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a anti-CD19 antibody, or a pharmaceutically acceptable form thereof, wherein the PI3K inhibitor is a PI3K delta inhibitor.

In one embodiment of the compositions and methods provided herein, the PI3K inhibitor is a PI3K delta inhibitor, and the anti-CD19 antibody is blinatumomab.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the anti-CD19 antibody (e.g., blinatumomab), or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the anti-CD19 antibody (e.g., blinatumomab), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the anti-CD19 antibody (e.g., blinatumomab) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a anti-CD19 antibody, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a anti-CD19 antibody, or a pharmaceutically acceptable form thereof, wherein the PI3K inhibitor is Idelalisib.

In one embodiment of the compositions and methods provided herein, the PI3K inhibitor is Idelalisib, and the anti-CD19 antibody is blinatumomab.

In one embodiment of the methods described herein, Idelalisib, or a pharmaceutically acceptable form thereof, is administered at a dosage of in the range of from about 0.01 mg to about 75 mg daily and the anti-CD19 antibody (e.g., blinatumomab), or a pharmaceutically acceptable form thereof, is administered at a dosage of in the range of from about 0.01 mg to about 1100 mg daily.

In one embodiment of the compositions and methods described herein, Idelalisib, or a pharmaceutically acceptable form thereof, to the anti-CD19 antibody (e.g., blinatumomab), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the anti-CD19 antibody (e.g., blinatumomab) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a anti-CD19 antibody, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a anti-CD19 antibody, or a pharmaceutically acceptable form thereof, wherein the PI3K inhibitor is a PI3K delta/gamma dual inhibitor.

In one embodiment of the compositions and methods provided herein, the PI3K inhibitor is a PI3K delta/gamma dual inhibitor, and the anti-CD19 antibody is blinatumomab.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the anti-CD19 antibody (e.g., blinatumomab), or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the anti-CD19 antibody (e.g., blinatumomab), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the anti-CD19 antibody (e.g., blinatumomab) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a anti-CD19 antibody, or a pharmaceutically acceptable form thereof, wherein the PI3K inhibitor is Compound 1:

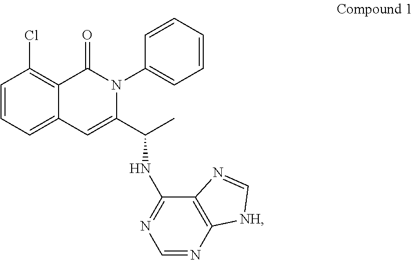 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a anti-CD19 antibody, or a pharmaceutically acceptable form thereof, wherein the PI3K inhibitor is Compound 1:

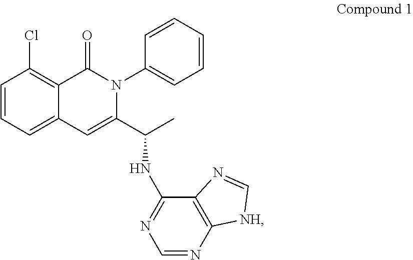 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the compositions and methods provided herein, the PI3K inhibitor is Compound 1, and the anti-CD19 antibody is blinatumomab.

In one embodiment of the methods described herein, Compound 1, or a pharmaceutically acceptable form thereof, is administered at a dosage of in the range of from about 0.01 mg to about 75 mg daily and the anti-CD19 antibody (e.g., blinatumomab), or a pharmaceutically acceptable form thereof, is administered at a dosage of in the range of from about 0.01 mg to about 1100 mg daily.

In one embodiment of the compositions and methods described herein, Compound 1, or a pharmaceutically acceptable form thereof, to the anti-CD19 antibody (e.g., blinatumomab), or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the anti-CD19 antibody (e.g., blinatumomab) is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment of the methods provided herein, the anti-CD19 antibody is blinatumomab, and it is administered intravenously. In one embodiment, blinatumomab is administered at a dose of from about 1 mg/kg to about 60 μg/m²/day, from about 1 mg/kg to about 50 μg/m²/day, from about 1 mg/kg to about 40 μg/m²/day, from about 1 mg/kg to about 30 μg/m²/day, from about 1 mg/kg to about 20 μg/m²/day, or from about 1 mg/kg to about 10 μg/m²/day. In one embodiment, blinatumomab is administered at a dose of about 60 μg/m²/day, about 50 μg/m²/day, about 40 μg/m²/day, about 30 μg/m²/day, about 20 μg/m²/day, or about 10 μg/m²/day. In one embodiment, the frequency of the administration of blinatumomab is reduced to once every 2 days, once every 3 days, once every week, or once every 2 week.

In one embodiment, the anti-CD19 antibody (e.g., blinatumomab), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the anti-CD19 antibody (e.g., blinatumomab), or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the anti-CD19 antibody (e.g., blinatumomab), or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In certain embodiments, the PI3K inhibitor (e.g., Compound 1), or a pharmaceutically acceptable form thereof, and the anti-CD19 antibody (e.g., blinatumomab) are administered via a same route. In other embodiments, the PI3K inhibitor (e.g., Compound 1), or a pharmaceutically acceptable form thereof, and the anti-CD19 antibody (e.g., blinatumomab) are administered via different routes, e.g., one is administered orally and the other is administered intravenously. In one embodiment, Compound 1 is administered orally and the anti-CD19 antibody (e.g., blinatumomab) is administered intravenously.

In certain embodiments, the PI3K inhibitor (e.g., Compound 1), or a pharmaceutically acceptable form thereof, and the anti-CD19 antibody (e.g., blinatumomab), or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and a anti-CD19 antibody (e.g., blinatumomab).

### 2.5 Combinations of PI3K Inhibitors and TLR Agonists

In certain embodiments, provided herein are methods of treating, managing, or preventing a cancer in a subject comprising administering to the subject a therapeutically effective amount of a PI3K inhibitor, or a pharmaceutically acceptable form thereof, in combination with a TLR agonist, or a pharmaceutically acceptable form thereof.

In certain embodiments, provided herein are pharmaceutical compositions comprising a therapeutically effective amount of a PI3K inhibitor, or a pharmaceutically acceptable form thereof, and a TLR agonist, or a pharmaceutically acceptable form thereof.

Toll-like receptors (TLRs) are a class of proteins that play a key role in the innate immune system. They are single, membrane-spanning, non-catalytic receptors usually expressed in sentinel cells such as macrophages and dendritic cells, that recognize structurally conserved molecules derived from microbes. Once these microbes have breached physical barriers such as the skin or intestinal tract mucosa, they are recognized by TLRs, which activate immune cell responses. The TLRs include TLR1, TLR2, TLR3, TLR4, TLR5, TLR6, TLR7, TLR8, TLR9, TLR10, TLR11, TLR12, and TLR13, though the latter two are not found in humans.

In one embodiment, provided herein is a method of treating, managing, or preventing a cancer in a subject comprising administering to the subject a therapeutically effective amount of a PI3K inhibitor, or a pharmaceutically acceptable form thereof, in combination with a TLR agonist, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a TLR agonist, or a pharmaceutically acceptable form thereof, wherein the PI3K inhibitor is a PI3K delta inhibitor.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the TLR agonist, or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the TLR agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the TLR agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a TLR agonist, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a TLR agonist, or a pharmaceutically acceptable form thereof, wherein the PI3K inhibitor is Idelalisib.

In one embodiment of the methods described herein, Idelalisib, or a pharmaceutically acceptable form thereof, is administered at a dosage of in the range of from about 0.01 mg to about 75 mg daily and the TLR agonist, or a pharmaceutically acceptable form thereof, is administered at a dosage of in the range of from about 0.01 mg to about 1100 mg daily.

In one embodiment of the compositions and methods described herein, Idelalisib, or a pharmaceutically acceptable form thereof, to the TLR agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the TLR agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a TLR agonist, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a TLR agonist, or a pharmaceutically acceptable form thereof, wherein the PI3K inhibitor is a PI3K delta/gamma dual inhibitor.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the TLR agonist, or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the TLR agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the TLR agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a TLR agonist, or a pharmaceutically acceptable form thereof, wherein the PI3K inhibitor is Compound 1:

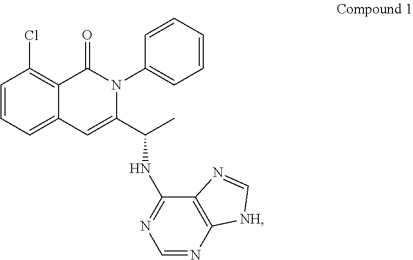 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a TLR agonist, or a pharmaceutically acceptable form thereof, wherein the PI3K inhibitor is Compound 1:

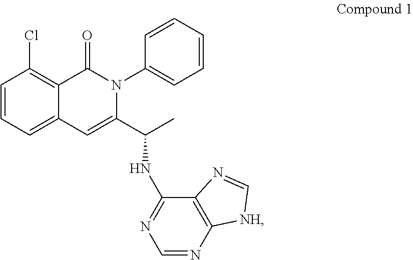 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the methods described herein, Compound 1, or a pharmaceutically acceptable form thereof, is administered at a dosage of in the range of from about 0.01 mg to about 75 mg daily and the TLR agonist, or a pharmaceutically acceptable form thereof, is administered at a dosage of in the range of from about 0.01 mg to about 1100 mg daily.

In one embodiment of the compositions and methods described herein, Compound 1, or a pharmaceutically acceptable form thereof, to the TLR agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the TLR agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the TLR agonist, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the TLR agonist, or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the TLR agonist, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In certain embodiments, the PI3K inhibitor (e.g., Compound 1), or a pharmaceutically acceptable form thereof, and the TLR agonist are administered via a same route, e.g., both are administered orally. In other embodiments, the PI3K inhibitor (e.g., Compound 1), or a pharmaceutically acceptable form thereof, and the TLR agonist are administered via different routes, e.g., one is administered orally and the other is administered intravenously. In one embodiment, Compound 1 is administered orally and the TLR agonist is administered intravenously.

In certain embodiments, the PI3K inhibitor (e.g., Compound 1), or a pharmaceutically acceptable form thereof, and the TLR agonist, or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and a TLR agonist.

### 2.6 Combinations of PI3K Inhibitors and STING Agonists

In certain embodiments, provided herein are methods of treating, managing, or preventing a cancer in a subject comprising administering to the subject a therapeutically effective amount of a PI3K inhibitor, or a pharmaceutically acceptable form thereof, in combination with a STING agonist, or a pharmaceutically acceptable form thereof.

In certain embodiments, provided herein are pharmaceutical compositions comprising a therapeutically effective amount of a PI3K inhibitor, or a pharmaceutically acceptable form thereof, and a STING agonist, or a pharmaceutically acceptable form thereof.

Stimulator of interferon genes (STING), also known as transmembrane protein 173 (TMEM173) and MPYS/MITA/ERIS, is a protein that in humans is encoded by the TMEM173 gene. STING plays an important role in innate immunity. STING induces type I interferon production when cells are infected with intracellular pathogens, such as viruses, mycobacteria and intracellular parasites. Type I interferon, mediated by STING, protects infected cells and nearby cells from local infection by binding to the same cell that secretes it (autocrine signaling) and nearby cells (paracrine signaling).

In one embodiment, provided herein is a method of treating, managing, or preventing a cancer in a subject comprising administering to the subject a therapeutically effective amount of a PI3K inhibitor, or a pharmaceutically acceptable form thereof, in combination with a STING agonist, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a STING agonist, or a pharmaceutically acceptable form thereof, wherein the PI3K inhibitor is a PI3K delta inhibitor.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the STING agonist, or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the STING agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the STING agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a STING agonist, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a STING agonist, or a pharmaceutically acceptable form thereof, wherein the PI3K inhibitor is Idelalisib.

In one embodiment of the methods described herein, Idelalisib, or a pharmaceutically acceptable form thereof, is administered at a dosage of in the range of from about 0.01 mg to about 75 mg daily and the STING agonist, or a pharmaceutically acceptable form thereof, is administered at a dosage of in the range of from about 0.01 mg to about 1100 mg daily.

In one embodiment of the compositions and methods described herein, Idelalisib, or a pharmaceutically acceptable form thereof, to the STING agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the STING agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a STING agonist, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a STING agonist, or a pharmaceutically acceptable form thereof, wherein the PI3K inhibitor is a PI3K delta/gamma dual inhibitor.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the STING agonist, or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the STING agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the STING agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a STING agonist, or a pharmaceutically acceptable form thereof, wherein the PI3K inhibitor is Compound 1:

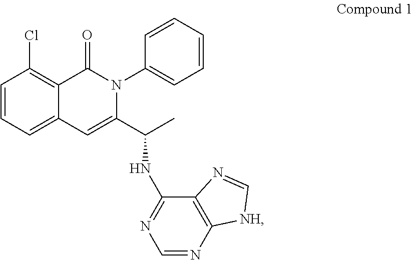 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a STING agonist, or a pharmaceutically acceptable form thereof, wherein the PI3K inhibitor is Compound 1:

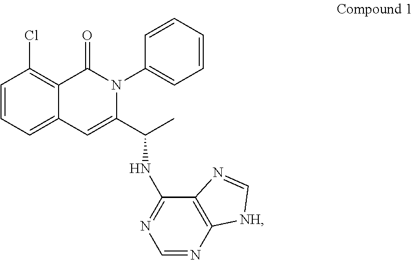 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the methods described herein, Compound 1, or a pharmaceutically acceptable form thereof, is administered at a dosage of in the range of from about 0.01 mg to about 75 mg daily and the STING agonist, or a pharmaceutically acceptable form thereof, is administered at a dosage of in the range of from about 0.01 mg to about 1100 mg daily.

In one embodiment of the compositions and methods described herein, Compound 1, or a pharmaceutically acceptable form thereof, to the STING agonist, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the STING agonist is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the STING agonist, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the STING agonist, or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the STING agonist, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In certain embodiments, the PI3K inhibitor (e.g., Compound 1), or a pharmaceutically acceptable form thereof, and the STING agonist are administered via a same route, e.g., both are administered orally. In other embodiments, the PI3K inhibitor (e.g., Compound 1), or a pharmaceutically acceptable form thereof, and the STING agonist are administered via different routes, e.g., one is administered orally and the other is administered intravenously. In one embodiment, Compound 1 is administered orally and the STING agonist is administered intravenously.

In certain embodiments, the PI3K inhibitor (e.g., Compound 1), or a pharmaceutically acceptable form thereof, and the STING agonist, or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and a STING agonist.

### 2.7 Combinations of P3K Inhibitors and Flt3 Ligands

In certain embodiments, provided herein are methods of treating, managing, or preventing a cancer in a subject comprising administering to the subject a therapeutically effective amount of a PI3K inhibitor, or a pharmaceutically acceptable form thereof, in combination with a Flt3 ligand, or a pharmaceutically acceptable form thereof.

In certain embodiments, provided herein are pharmaceutical compositions comprising a therapeutically effective amount of a PI3K inhibitor, or a pharmaceutically acceptable form thereof, and a Flt3 ligand, or a pharmaceutically acceptable form thereof.

Fms-related tyrosine kinase 3 ligand (FLT3LG) is a protein which in humans is encoded by the FLT3LG gene. Flt3 ligand is a hematopoietic four helical bundle cytokine. It is structurally homologous to stem cell factor (SCF) and colony stimulating factor 1 (CSF-1). In synergy with other growth factors, Flt3 ligand stimulates the proliferation and differentiation of various blood cell progenitors.

In one embodiment, provided herein is a method of treating, managing, or preventing a cancer in a subject comprising administering to the subject a therapeutically effective amount of a PI3K inhibitor, or a pharmaceutically acceptable form thereof, in combination with a Flt3 ligand, or a pharmaceutically acceptable form thereof, wherein the PI3K inhibitor is a PI3K delta inhibitor.

In one embodiment, provided herein is a pharmaceutical composition comprising a therapeutically effective amount of a PI3K inhibitor, or a pharmaceutically acceptable form thereof, and a Flt3 ligand, or a pharmaceutically acceptable form thereof, wherein the PI3K inhibitor is a PI3K delta inhibitor.

In one embodiment of the methods described herein, the PI3K delta inhibitor (e.g., Idelalisib), or a pharmaceutically acceptable form thereof, is administered at a dosage of in the range of from about 0.01 mg to about 75 mg daily and the Flt3 ligand, or a pharmaceutically acceptable form thereof, is administered at a dosage of in the range of from about 0.01 mg to about 1100 mg daily.

In one embodiment of the compositions and methods described herein, the molar ratio of the PI3K delta inhibitor (e.g., Idelalisib), or a pharmaceutically acceptable form thereof, to the Flt3 ligand, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta inhibitor (e.g., Idelalisib) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the Flt3 ligand is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a Flt3 ligand, or a pharmaceutically acceptable form thereof, wherein the PI3K inhibitor is Idelalisib.

In one embodiment, provided herein is a pharmaceutical composition comprising a therapeutically effective amount of a PI3K inhibitor, or a pharmaceutically acceptable form thereof, and a Flt3 ligand, or a pharmaceutically acceptable form thereof, wherein the PI3K inhibitor is Idelalisib.

In one embodiment of the methods described herein, Idelalisib, or a pharmaceutically acceptable form thereof, is administered at a dosage of in the range of from about 0.01 mg to about 75 mg daily and the Flt3 ligand, or a pharmaceutically acceptable form thereof, is administered at a dosage of in the range of from about 0.01 mg to about 1100 mg daily.

In one embodiment of the compositions and methods described herein, Idelalisib, or a pharmaceutically acceptable form thereof, to the Flt3 ligand, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Idelalisib is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the Flt3 ligand is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a Flt3 ligand, or a pharmaceutically acceptable form thereof, wherein the PI3K inhibitor is a PI3K delta/gamma dual inhibitor.

In one embodiment, provided herein is a pharmaceutical composition comprising a therapeutically effective amount of a PI3K inhibitor, or a pharmaceutically acceptable form thereof, and a Flt3 ligand, or a pharmaceutically acceptable form thereof, wherein the PI3K inhibitor is a PI3K delta/gamma dual inhibitor.

In one embodiment of the methods described herein, the PI3K delta/gamma dual inhibitor (e.g., Compound 1), or a pharmaceutically acceptable form thereof, is administered at a dosage of in the range of from about 0.01 mg to about 75 mg daily and the Flt3 ligand, or a pharmaceutically acceptable form thereof, is administered at a dosage of in the range of from about 0.01 mg to about 1100 mg daily.

In one embodiment of the compositions and methods described herein, the molar ratio of the PI3K delta/gamma dual inhibitor (e.g., Compound 1), or a pharmaceutically acceptable form thereof, to the Flt3 ligand, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, the PI3K delta/gamma dual inhibitor (e.g., Compound 1) is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the Flt3 ligand is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, provided herein is a method of treating, managing, or preventing a cancer in a subject comprising administering to the subject a therapeutically effective amount of a PI3K inhibitor, or a pharmaceutically acceptable form thereof, in combination with a Flt3 ligand, or a pharmaceutically acceptable form thereof, wherein the PI3K inhibitor is Compound 1:

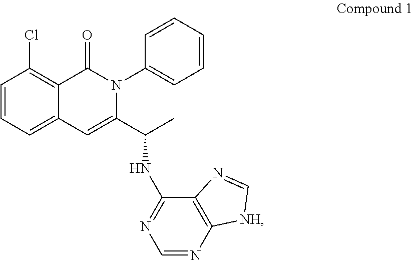 [SMILES: C[C@H](Nc1ncnc2[nH]cnc12)c1cc2cccc(Cl)c2c(=O)n1-c1ccccc1]

or a pharmaceutically acceptable form thereof.

In one embodiment, provided herein is a pharmaceutical composition comprising a therapeutically effective amount of a PI3K inhibitor, or a pharmaceutically acceptable form thereof, and a Flt3 ligand, or a pharmaceutically acceptable form thereof, wherein the PI3K inhibitor is Compound 1:

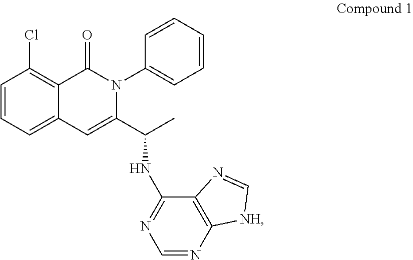 [SMILES: C[C@H](Nc1ncnc2[nH]cnc12)c1cc2cccc(Cl)c2c(=O)n1-c1ccccc1]

or a pharmaceutically acceptable form thereof.

In one embodiment of the methods described herein, Compound 1, or a pharmaceutically acceptable form thereof, is administered at a dosage of in the range of from about 0.01 mg to about 75 mg daily and the Flt3 ligand, or a pharmaceutically acceptable form thereof, is administered at a dosage of in the range of from about 0.01 mg to about 1100 mg daily.

In one embodiment of the compositions and methods described herein, Compound 1, or a pharmaceutically acceptable form thereof, to the Flt3 ligand, or a pharmaceutically acceptable form thereof, is in the range of from about 500:1 to about 1:500, from about 400:1 to about 1:400, from about 300:1 to about 1:300, from about 200:1 to about 1:200, from about 100:1 to about 1:100, from about 75:1 to about 1:75, from about 50:1 to about 1:50, from about 40:1 to about 1:40, from about 30:1 to about 1:30, from about 20:1 to about 1:20, from about 10:1 to about 1:10, from about 5:1 to about 1:5.

In one embodiment, Compound 1 is administered at an amount that is decreased by about 1.5 fold to about 50 fold, about 1.5 fold to about 25 fold, about 1.5 fold to about 20 fold, about 1.5 fold to about 15 fold, about 1.5 fold to about 10 fold, about 2 fold to about 10 fold, about 2 fold to about 8 fold, about 4 fold to about 6 fold, or about 5 fold of the amount when administered individually; and
the Flt3 ligand is administered at an amount that is decreased by about 1.1 fold to about 50 fold, about 1.1 fold to about 40 fold, about 1.1 fold to about 30 fold, about 1.1 fold to about 25 fold, about 1.1 fold to about 20 fold, about 1.1 fold to about 15 fold, about 1.1 fold to about 10 fold of the amount when administered individually.

In one embodiment, the Flt3 ligand, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before the PI3K inhibitor (e.g., Compound 1), or a pharmaceutically acceptable form thereof, is administered. In another embodiment, the Flt3 ligand, or a pharmaceutically acceptable form thereof, is administered concurrently with the PI3K inhibitor (e.g., Compound 1), or a pharmaceutically acceptable form thereof, in a single dosage form or separate dosage forms. In yet another embodiment, the Flt3 ligand, or a pharmaceutically acceptable form thereof, is administered to the subject at least 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after the PI3K inhibitor (e.g., Compound 1), or a pharmaceutically acceptable form thereof, is administered.

In certain embodiments, the PI3K inhibitor (e.g., Compound 1), or a pharmaceutically acceptable form thereof, and the Flt3 ligand are administered via a same route, e.g., both are administered orally. In other embodiments, the PI3K inhibitor (e.g., Compound 1), or a pharmaceutically acceptable form thereof, and the Flt3 ligand are administered via different routes, e.g., one is administered orally and the other is administered intravenously. In one embodiment, Compound 1 is administered orally and the Flt3 ligand is administered intravenously.

In certain embodiments, the PI3K inhibitor (e.g., Compound 1), or a pharmaceutically acceptable form thereof, and the Flt3 ligand, or a pharmaceutically acceptable form thereof, are the only therapeutically active ingredients of the compositions and methods provided herein. In other embodiments, the compositions provided herein comprise and the methods provided herein use at least one more therapeutically active ingredient. In one embodiment, the compositions provided herein comprise and the methods provided herein use a PI3K delta inhibitor (e.g., Idelalisib), a PI3K delta/gamma dual inhibitor, and a Flt3 ligand.

### Cancers

The diseases or disorders (e.g., cancer) that can be treated, managed, or prevented with a pharmaceutical composition as provided herein, or according to the methods as provided herein, include, but are not limited to, breast cancer such as a ductal carcinoma, lobular carcinoma, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, NK cell leukemia (e.g., blastic plasmacytoid dendritic cell neoplasm), acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer; kidney cancer; thyroid cancer such as papillary, follicular, medullary and anaplastic; lymphoma such as diffuse large B-cell lymphoma, B-cell immunoblastic lymphoma, NK cell lymphoma (e.g., blastic plasmacytoid dendritic cell neoplasm), and Burkitt lymphoma; Kaposi's Sarcoma; viral-induced cancers including hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), oligodendroglioma, ependymoma, meningioma, lymphoma, schwannoma, and medulloblastoma; peripheral nervous system (PNS) cancers such as acoustic neuromas and malignant peripheral nerve sheath tumor (MPNST) including neurofibromas and schwannomas, malignant fibrocytoma, malignant fibrous histiocytoma, malignant meningioma, malignant mesothelioma, and malignant mixed Millerian tumor; oral cavity and oropharyngeal cancers such as, hypopharyngeal cancer, laryngeal cancer, nasopharyngeal cancer, and oropharyngeal cancer; stomach cancers such as lymphomas, gastric stromal tumors, and carcinoid tumors; testicular cancers such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin lymphoma, non-Hodgkin lymphomas carcinoids or carcinoid tumors; rectal cancer; and colon cancer.

In one embodiment, the cancer or disease is a blood disorder or a hematologic malignancy.

In some embodiments, the cancer or disease is selected from one or more of the following: acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), which includes B-lineage ALL and T-lineage ALL, chronic lymphocytic leukemia (CLL), prolymphocytic leukemia (PLL), hairy cell leukemia (HCL) and Waldenstrom's macroglobulinemia (WM); peripheral T cell lymphomas (PTCL), adult T cell leukemia/lymphoma (ATL), cutaneous T-cell lymphoma (CTCL), large granular lymphocytic leukemia (LGF), Hodgkin's disease and Reed-Stemberg disease; acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic andenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenstrom's macroglobulinemia.

In one embodiment, the cancer or disease provided herein, such as a blood disorder or hematologic malignancy, has a high expression level of one or more PI3K isoform(s) (e.g., PI3K-α, PI3K-β, PI3K-δ, or PI3K-γ, or a combination thereof).

In one embodiment, the cancer or disease is a blood disorder or a hematologic malignancy, including, but not limited to, myeloid disorder, lymphoid disorder, leukemia, lymphoma, myelodysplastic syndrome (MDS), myeloproliferative disease (MPD), mast cell disorder, and myeloma (e.g., multiple myeloma), among others.

In one embodiment, the blood disorder or the hematologic malignancy includes, but is not limited to, acute lymphoblastic leukemia (ALL), T-cell ALL (T-ALL), B-cell ALL (B-ALL), acute myeloid leukemia (AML), chronic lymphocytic leukemia (CLL), chronic myelogenous leukemia (CML), blast phase CML, small lymphocytic lymphoma (SLL), CLL/SLL, blast phase CLL, Hodgkin lymphoma (HL), non-Hodgkin lymphoma (NHL), B-cell NHL, T-cell NHL, indolent NHL (iNHL), diffuse large B-cell lymphoma (DLBCL), mantle cell lymphoma (MCL), aggressive B-cell NHL, B-cell lymphoma (BCL), Richter's syndrome (RS), T-cell lymphoma (TCL), peripheral T-cell lymphoma (PTCL), cutaneous T-cell lymphoma (CTCL), transformed mycosis fungoides, Sézary syndrome, anaplastic large-cell lymphoma (ALCL), follicular lymphoma (FL), Waldenstrom macroglobulinemia (WM), lymphoplasmacytic lymphoma, Burkitt lymphoma, multiple myeloma (MM), amyloidosis, MPD, essential thrombocytosis (ET), myelofibrosis (MF), polycythemia vera (PV), chronic myelomonocytic leukemia (CMML), myelodysplastic syndrome (MDS), angioimmunoblastic lymphoma, high-risk MDS, and low-risk MDS. In one embodiment, the hematologic malignancy is relapsed. In one embodiment, the hematologic malignancy is refractory. In one embodiment, the cancer or disease is in a pediatric patient (including an infantile patient). In one embodiment, the cancer or disease is in an adult patient. Additional embodiments of a cancer or disease being treated or prevented by methods, compositions, or kits provided herein are described herein elsewhere.

In exemplary embodiments, the cancer or hematologic malignancy is CLL. In exemplary embodiments, the cancer or hematologic malignancy is CLL/SLL. In exemplary embodiments, the cancer or hematologic malignancy is blast phase CLL. In exemplary embodiments, the cancer or hematologic malignancy is SLL.

In exemplary embodiments, the cancer or hematologic malignancy is iNHL. In exemplary embodiments, the cancer or hematologic malignancy is DLBCL. In exemplary embodiments, the cancer or hematologic malignancy is B-cell NHL (e.g., aggressive B-cell NHL). In exemplary embodiments, the cancer or hematologic malignancy is MCL. In exemplary embodiments, the cancer or hematologic malignancy is RS. In exemplary embodiments, the cancer or hematologic malignancy is AML. In exemplary embodiments, the cancer or hematologic malignancy is MM. In exemplary embodiments, the cancer or hematologic malignancy is ALL. In exemplary embodiments, the cancer or hematologic malignancy is T-ALL. In exemplary embodiments, the cancer or hematologic malignancy is B-ALL. In exemplary embodiments, the cancer or hematologic malignancy is TCL. In exemplary embodiments, the cancer or hematologic malignancy is ALCL. In exemplary embodiments, the cancer or hematologic malignancy is leukemia. In exemplary embodiments, the cancer or hematologic malignancy is lymphoma. In exemplary embodiments, the cancer or hematologic malignancy is T-cell lymphoma. In exemplary embodiments, the cancer or hematologic malignancy is MDS (e.g., low grade MDS). In exemplary embodiments, the cancer or hematologic malignancy is MPD. In exemplary embodiments, the cancer or hematologic malignancy is a mast cell disorder. In exemplary embodiments, the cancer or hematologic malignancy is Hodgkin lymphoma (HL). In exemplary embodiments, the cancer or hematologic malignancy is non-Hodgkin lymphoma. In exemplary embodiments, the cancer or hematologic malignancy is PTCL. In exemplary embodiments, the cancer or hematologic malignancy is CTCL (e.g., mycosis fungoides or Sézary syndrome). In exemplary embodiments, the cancer or hematologic malignancy is WM. In exemplary embodiments, the cancer or hematologic malignancy is CML. In exemplary embodiments, the cancer or hematologic malignancy is FL. In exemplary embodiments, the cancer or hematologic malignancy is transformed mycosis fungoides. In exemplary embodiments, the cancer or hematologic malignancy is Sézary syndrome. In exemplary embodiments, the cancer or hematologic malignancy is acute T-cell leukemia. In exemplary embodiments, the cancer or hematologic malignancy is acute B-cell leukemia. In exemplary embodiments, the cancer or hematologic malignancy is Burkitt lymphoma. In exemplary embodiments, the cancer or hematologic malignancy is myeloproliferative neoplasms. In exemplary embodiments, the cancer or hematologic malignancy is splenic marginal zone. In exemplary embodiments, the cancer or hematologic malignancy is nodal marginal zone. In exemplary embodiments, the cancer or hematologic malignancy is extranodal marginal zone.

In one embodiment, the cancer or hematologic malignancy is a B cell lymphoma. In a specific embodiment, provided herein is a method of treating or managing a B cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a B cell lymphoma comprising administering to a patient a therapeutically effective amount of a compound provided herein, or a pharmaceutically acceptable derivative (e.g., salt or solvate) thereof. In one embodiment, the B cell lymphoma is iNHL. In another embodiment, the B cell lymphoma is follicular lymphoma. In another embodiment, the B cell lymphoma is Waldenstrom macroglobulinemia (lymphoplasmacytic lymphoma). In another embodiment, the B cell lymphoma is marginal zone lymphoma (MZL). In another embodiment, the B cell lymphoma is MCL. In another embodiment, the B cell lymphoma is HL. In another embodiment, the B cell lymphoma is aNHL. In another embodiment, the B cell lymphoma is DLBCL. In another embodiment, the B cell lymphoma is Richters lymphoma.

In one embodiment, the cancer or hematologic malignancy is a T cell lymphoma. In a specific embodiment, provided herein is a method of treating or managing a T cell lymphoma comprising administering to a patient a therapeutically effective amount of a compound provided herein, or a pharmaceutically acceptable derivative (e.g., salt or solvate) thereof. Also provided herein is a method of treating or lessening one or more of the symptoms associated with a T cell lymphoma comprising administering to a patient a therapeutically effective amount of a compound provided herein, or a pharmaceutically acceptable derivative (e.g., salt or solvate) thereof. In one embodiment, the T cell lymphoma is peripheral T cell lymphoma (PTCL). In another embodiment, the T cell lymphoma is cutaneous T cell lymphoma (CTCL).

In one embodiment, the cancer or hematologic malignancy is Sézary syndrome. In a specific embodiment, provided herein is a method of treating or managing Sézary syndrome comprising administering to a patient a therapeutically effective amount of a compound provided herein, or a pharmaceutically acceptable derivative (e.g., salt or solvate) thereof. Also provided herein is a method of treating or lessening one or more of the symptoms associated with Sézary syndrome comprising administering to a patient a therapeutically effective amount of a compound provided herein, or a pharmaceutically acceptable derivative (e.g., salt or solvate) thereof. The symptoms associated with Sézary syndrome include, but are not limited to, epidermotropism by neoplastic CD4+lymphocytes, Pautrier's microabscesses, erythroderma, lymphadenopathy, atypical T cells in the peripheral blood, and hepatosplenomegaly.

The effectiveness of treatment in the preceding methods can for example be determined by measuring the decrease in size of tumors present in the patients with the neoplastic condition, or by assaying a molecular determinant of the degree of proliferation of the tumor cells.

Suitable test agents which can be tested in the preceding method include combinatorial libraries, defined chemical entities, peptide and peptide mimetics, oligonucleotides and natural product libraries, such as display (e.g. phage display libraries) and antibody products. Test agents may be used in an initial screen of, for example, 10 substances per reaction, and the substances of these batches which show inhibition or activation tested individually. Test agents may be used at a concentration of from 1 nM to 1000 μM, preferably from 1 μM to 100 μM, more preferably from 1 μM to 10M.

In certain embodiments, provided herein is a method of treating, managing, or preventing a cancer in a subject comprising administering to the subject a PI3K inhibitor (e.g., one or more PI3K inhibitors, e.g., Idelalisib and/or Compound 1), or a pharmaceutically acceptable form thereof, in combination with a second agent or a pharmaceutically acceptable form thereof, wherein the second agent is selected from one or more of 1) a checkpoint modulator, 2) an XPO1 inhibitor, 3) an anti-CD19 antibody, 4) a TLR agonist, 5) a STING agonist, or 6) a Flt3 ligand, wherein the cancer is diffuse large B-cell lymphoma (activated B-cell-like), diffuse large B-cell lymphoma (germinal center B-cell-like), follicular lymphoma, indolent non-Hodgkin lymphoma, T-cell lymphoma, mantle cell lymphoma, or multiple myeloma. In certain embodiments, the combination is therapeutically effective. In certain embodiments, the combination is synergistic.

In one embodiment of the methods provided herein, the subject shows decreased responsiveness to a PI3K inhibitor (e.g., is resistant or refractive to treatment with a PI3K inhibitor, e.g., Compound 1). In one embodiment, the subject is identified as having a decreased susceptibility (e.g., resistance or acquired resistance) to a monotherapy treatment of a PI3K inhibitor (e.g., Compound 1), or a pharmaceutically acceptable form thereof. In one embodiment, the subject is identified as having an increased susceptibility to a combination therapy treatment provided herein.

Also provided herein are methods of delaying resistance of a subject, or prolonging remission (e.g., complete remission or partial remission) of a subject, to a PI3K inhibitor, e.g., Compound 1 or CAL-101 or to a second agent such as a checkpoint modulator, an XPO1 inhibitor, an anti-CD19 antibody, a TLR agonist, a STING agonist, or a Flt3 ligand described herein. In some embodiments, the method of delaying resistance of the subject, or prolonging remission (e.g., complete remission or partial remission) of the subject, comprises administering a combination of a PI3K inhibitor (e.g., Compound 1 or CAL-101) and a second agent (e.g., a checkpoint modulator, an XPO1 inhibitor, an anti-CD19 antibody, a TLR agonist, a STING agonist, or a Flt3 ligand described herein to the subject before the subject develops resistance to the PI3K inhibitor (e.g., Compound 1 or CAL-101). In some embodiments, the method of delaying resistance of the subject, or prolonging remission (e.g., complete remission or partial remission) of the subject, comprises administering a combination of a PI3K inhibitor (e.g., Compound 1 or CAL-101) and a second agent (e.g., a checkpoint modulator, an XPO1 inhibitor, an anti-CD19 antibody, a TLR agonist, a STING agonist, or a Flt3 ligand described herein) to the subject before the subject develops resistance to the second agent.

In some embodiments, the subject is not resistant to a PI3K inhibitor (e.g., Compound 1 or CAL-101). In some embodiments, the subject is not resistant to a checkpoint modulator, an XPO1 inhibitor, an anti-CD19 antibody, a TLR agonist, a STING agonist, or a Flt3 ligand described herein. In some embodiments, the subject has previously been administered a PI3K inhibitor (e.g., Compound 1 or CAL-101) as a monotherapy or in combination with an agent other than a checkpoint modulator, an XPO1 inhibitor, an anti-CD19 antibody, a TLR agonist, a STING agonist, or a Flt3 ligand described herein. In some embodiments, the subject has previously been administered a checkpoint modulator, an XPO1 inhibitor, an anti-CD19 antibody, a TLR agonist, a STING agonist, or a Flt3 ligand described herein as a monotherapy or in combination with an agent other than a checkpoint modulator, an XPO1 inhibitor, an anti-CD19 antibody, a TLR agonist, a STING agonist, or a Flt3 ligand described herein. In some embodiments, the subject has a cancer, e.g., a cancer described herein. In some embodiments, in accordance with the method, resistance is delayed compared to the time in which resistance generally develops when the subject is treated with any of the agents or inhibitors alone as monotherapy. In some embodiments, the resistance is delayed by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more. In some embodiments, in accordance with the method, remission (e.g., complete remission or partial remission) is prolonged compared to the time in which remission generally lasts when the subject is treated with any of the agents or inhibitors alone as monotherapy. In some embodiments, remission (e.g., complete remission or partial remission) is prolonged by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years, or more.

In some embodiments, once the subject becomes resistant to the PI3K inhibitor (e.g., Compound 1 or CAL-101) or the second agent (e.g., a checkpoint modulator, an XPO1 inhibitor, an anti-CD19 antibody, a TLR agonist, a STING agonist, or a Flt3 ligand described herein), the agent to which the subject is resistant is withdrawn. In other embodiments, once the subject becomes resistant to the PI3K inhibitor (e.g., Compound 1 or CAL-101) or the second agent (e.g., a checkpoint modulator, an XPO1 inhibitor, an anti-CD19 antibody, a TLR agonist, a STING agonist, or a Flt3 ligand described herein), the agent to which the subject is resistant continued. In some embodiments, addition of the PI3K inhibitor or the second agent to the therapeutic regimen increases or restores sensitivity to the agent to which the cancer is resistant. For instance, in some embodiments, addition of the second agent to the therapeutic regimen increases or restores sensitivity to the PI3K inhibitor to which the cancer is resistant.

Provided herein is also a method of reducing, e.g., overcoming, resistance of a subject to a PI3K inhibitor (e.g., Compound 1 or CAL-101), comprising administering the PI3K inhibitor as a monotherapy to the subject until development of resistance in the subject to the PI3K inhibitor, and subsequently administering a second agent (e.g., a checkpoint modulator, an XPO1 inhibitor, an anti-CD19 antibody, a TLR agonist, a STING agonist, or a Flt3 ligand described herein) to the subject. In some cases, the method comprises continuing administration of the PI3K inhibitor (e.g., at the same dosage, lower dosage, or higher dosage) to the subject in combination with the second agent. In other cases, the method comprises discontinuing administration of the PI3K inhibitor upon commencing administration of the second agent. For example the administration of the PI3K inhibitor is stopped before administration of the second agent commences. In other examples, the dosage of the PI3K inhibitor is decreased, e.g., gradually, upon commencing administration of the second agent. In some embodiments, provided herein is a method of reducing, e.g., overcoming, resistance of a subject to a PI3K inhibitor (e.g., Compound 1 or CAL-101), comprising administering the PI3K inhibitor and the second agent (e.g., a checkpoint modulator, an XPO1 inhibitor, an anti-CD19 antibody, a TLR agonist, a STING agonist, or a Flt3 ligand described herein) to the subject before the subject develops resistance to the PI3K inhibitor, in order to prevent resistance arising, reduce the likelihood of resistance developing, or increase the length of time before resistance develops.

In one embodiment, a method described herein further comprises administration of a third agent of a CD20 inhibitor, e.g., an anti-CD20 antibody, in addition to the PI3K inhibitor and the second agent provided herein. In one embodiment, a pharmaceutical composition described herein further comprises a third agent of a CD20 inhibitor, e.g., an anti-CD20 antibody, in addition to the PI3K inhibitor and the second agent provided herein. In some such embodiments, the CD20 inhibitor, e.g., the anti-CD20 antibody, is included in the same dosage form as the PI3K inhibitor and/or second agent. In some such embodiments, the CD20 inhibitor, e.g., the anti-CD20 antibody, is in a separate dosage form as the PI3K inhibitor and/or second agent. The CD20 inhibitor, e.g., the anti-CD20 antibody, can be administered before, after, or concurrent with the PI3K inhibitor and/or second agent. Exemplary CD20 inhibitors include, but are not limited to, anti-CD20 antibody and other inhibitors, such as rituximab, obinutuzumab (GA-101), tositumomab, ³¹¹ tositumomab, ⁹⁰Y ibritumomab, ¹¹¹I ibritumomab, ofatumumab, veltuzumab, and ocrelizumab), AME-133v, PRO131921 and TRU-015.

The combination of the PI3K inhibitor and the third agent, e.g., a CD20 inhibitor, e.g., an anti-CD20 antibody, can be administered together in a single dosage form or administered separately in two or more different dosage forms as described herein. In certain embodiments, the anti-CD20 antibody is selected from rituximab, ofatumumab and obinutuzumab.

In an embodiment, a composition described herein includes a combination of a PI3K inhibitor (e.g., a PI3K inhibitor described herein, e.g., Compound 1 or CAL-101), a second agent provided herein, and a third agent of an anti-CD20 antibody or fragment thereof, e.g., an anti-CD20 monoclonal antibody (mAb), such as obinutuzumab. In some embodiments, provided herein is a method of treating, managing, or preventing a cancer in a subject comprising administering to the subject a combination of a PI3K inhibitor (e.g., Compound 1 or CAL-101), a second agent provided herein, in combination with an anti-CD20 antibody or fragment thereof, e.g., an anti-CD20 monoclonal antibody (mAb), such as obinutuzumab. In some embodiments, the subject has a cancer, e.g., a cancer described herein, e.g., a hematological cancer, such as a lymphoma. In some embodiments, the effect of combining the Compound 1 or CAL-101, a second agent provided herein, with obinutuzumab includes an additive effect on cell killing, e.g., cancer cell killing. In some embodiments, the PI3K inhibitor (e.g., Compound 1 or CAL-101) is administered concurrently with, prior to, or subsequent to, the obinutuzumab. In some embodiments, combinations of the PI3K inhibitor (e.g., Compound 1 or CAL-101), the second agent, and obinutuzumab allows the PI3K inhibitor, the second agent, and/or the obinutuzumab to be administered at a lower dosage or a lower frequency than would be required to achieve the same therapeutic effect compared to a monotherapy dose. Such a combination provides advantageous effects, e.g., in reducing, preventing, delaying, and/or decreasing the occurrence of one or more of: a side effect, toxicity, or resistance that would otherwise be associated with administration of a higher dose of one or both of the agents.

As a monotherapy, obinutuzumab can be administered according to the following regimen of 28-day cycles: 100 mg on C1D1 (cycle 1, day one), 900 mg on C1D2, 1000 mg on C1D8, 1000 mg on C1D15, and 1000 mg on day 1 of each subsequent cycle, e.g., cycles 2-6. In some embodiments, when administered in combination with a PI3K inhibitor and a second agent provided herein, the dosage of obinutuzumab can be reduced compared to its monotherapy dose, e.g., 300-400, 400-500, 500-600, 600-700, 700-800, 800-900, or 900-1000 mg/cycle (e.g., for a 28-day cycle). In some embodiments, when administered in combination with a PI3K inhibitor, the frequency of administration of obinutuzumab can be reduced compared to its frequency as a monotherapy, e.g., to one administration every 28-30, 30-35, 35-40, 40-45, 45-50, 50-55, or 55-60 days.

Methods for monitoring minimal residual disease negativity (MRD) are known in the art. See, e.g., Zhou, J. et al., Blood, 2007, 110: 1607-1611. Such methods include DNA based tests or RNA based tests. In certain embodiments, MRD is monitored using flow cytometry, sequencing, or PCR.

In some embodiments, the compositions and methods described herein are effective to reduce MRD.

In some embodiments, the methods described herein include selecting a subject for treatment with the combination of a PI3K inhibitor and the second agent. In certain embodiments, the subject (e.g., a patient with a cancer, e.g., a cancer described herein) is selected for treatment with the combination based on the MRD in the subject. In certain embodiments, the selection is based on the presence of an MRD above a preselected level (e.g., 1 malignant cell in 100 normal cells, 1 malignant cell in 1000 normal cells, or 1 malignant cell in 10,000 normal cells).

In some embodiments, the methods described herein further comprise monitoring the MRD in a subject, e.g., evaluating MRD at at least one, two, three, four, five, six, nine months after initiating, continuing or ceasing treatment (e.g., PI3K inhibitor monotherapy or a second agent monotherapy, or a combination therapy disclosed herein).

In some embodiments, the combination of a PI3K inhibitor (e.g. a PI3K inhibitor described herein) and a second agent (e.g., a second agent described herein) is effective to reduce the MRD in the subject, e.g., below a level previously measured in the subject (e.g., the level measured before the combination treatment). In certain embodiments, the combination of a PI3K inhibitor and a second agent is effective to reduce the MRD in the subject below the level observed during or after treatment with a monotherapy, e.g., a monotherapy comprising either the PI3K inhibitor or the second agent inhibitor. In certain embodiments, the MRD is decreased below the level observed during treatment with a monotherapy comprising the PI3K inhibitor. In certain embodiments, the MRD is decreased below the level observed during treatment with a monotherapy comprising the PI3K inhibitor.

In certain embodiments, the combination is effective to reduce the MRD below a preselected cutoff value (e.g., 1 malignant cell in 100 normal cells, 1 malignant cell in 1000 normal cells, or 1 malignant cell in 10,000 normal cells). In certain embodiments, the preselected cutoff value is 1 malignant cell in 1000 normal cells. In those embodiments where the MRD is below a preselected cutoff value (e.g., preselected cutoff value as described herein), the treatment (e.g., PI3K inhibitor monotherapy or a second agent monotherapy, or a combination therapy disclosed herein) can be altered or discontinued. If upon monitoring the MRD (at at least one, two, three, four, five, six, nine months after altering or discontinuing the therapy), the MRD levels are increased above a preselected cutoff (e.g., a preselected cutoff as described herein), a second treatment can be initiated (e.g., PI3K inhibitor monotherapy or the second agent monotherapy, a combination therapy disclosed herein, or a combination with a third agent, e.g., an anti-CD20 inhibitor or a BTK inhibitor such as ibrutinib).

In some embodiments provided herein is a method of treating cancer in a subject, the method comprising (i) administering to the subject a monotherapy (e.g., a monotherapy comprising a PI3K inhibitor or a second therapeutic agent as described herein) and monitoring the MRD in the subject, and (ii) if the MRD increases above a preselected cutoff value (e.g., 1 malignant cell in 100 normal cells, 1 malignant cell in 1000 normal cells, or 1 malignant cell in 10,000 normal cells), administering to the subject a PI3K inhibitor in combination with a second agent. In certain embodiments, the combination is effective to reduce the MRD, e.g. to reduce the MRD below the cutoff value. In certain embodiments, the preselected cutoff value is 1 malignant cell in 1000 or 10,000 normal cells.

In certain embodiments, provided herein is a method of decreasing minimal residual disease (MRD) in a subject diagnosed with a cancer, the method comprising: (a) administering to the subject a PI3K inhibitor (e.g., Compound 1), or a pharmaceutically acceptable form thereof, in combination with a second agent (e.g., at least one second agent); (b) monitoring the MRD in the subject by one or more methods described herein or known in the art (e.g., flow cytometry, sequencing, or PCR), and administering a monotherapy comprising the PI3K inhibitor, or a pharmaceutically acceptable form thereof, to the subject if the MRD in the subject increases above a preselected cutoff value (e.g., 1 malignant cell in 100 normal cells, 1 malignant cell in 1000 normal cells, or 1 malignant cell in 10,000 normal cells); and (c) monitoring the amount of MRD negativity (by one or more methods described herein or known in the art (e.g., flow cytometry, sequencing, or PCR) in the subject receiving the monotherapy, and administering a further combination comprising the PI3K inhibitor, or a pharmaceutically acceptable form thereof, and a third agent (e.g., at least one third agent) to the subject if the MRD is greater than the preselected cutoff value. In one embodiment, the third agent is selected from one or more of an anti-CD20 antibody, a MEK inhibitor, dexamethasone, lenolidomide, an mTOR inhibitor, nitrogen mustard, and a nucleoside metabolic inhibitor.

In some embodiments, the third agent is a chemotherapeutic. In some embodiments, the chemotherapeutic is selected from mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (imatinib mesylate), Velcade® (bortezomib), Casodex™ (bicalutamide), Iressa® (gefitinib), Tarceva® (erlotinib), and Adriamycin® (doxorubicin) as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; BTK inhibitors such as ibrutinib (PCI-32765), AVL-292, Dasatinib, LFM-A13, ONO-WG-307, and GDC-0834; HDAC inhibitors such as vorinostat, romidepsin, panobinostat, valproic acid, belinostat, mocetinostat, abrexinostat, entinostat, SB939, resminostat, givinostat, CUDC-101, AR-42, CHR-2845, CHR-3996, 4SC-202, CG200745, ACY-1215 and kevetrin; EZH2 inhibitors such as, but not limited to, EPZ-6438 (N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide), GSK-126 ((S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide), GSK-343 (1-Isopropyl-N-((6-methyl-2-oxo-4-propyl-1,2-dihydropyridin-3-yl)methyl)-6-(2-(4-methylpiperazin-1-yl)pyridine-4-yl)-1H-indazole-4-carboxamide), Eli, 3-deazaneplanocin A (DNNep, 5R-(4-amino-1H-imidazo[4,5-c]pyridin-1-yl)-3-(hydroxymethyl)-3-cyclopentene-1S,2R-diol), small interfering RNA (siRNA) duplexes targeted against EZH2 (S. M. Elbashir et al., Nature 411:494-498 (2001)), isoliquiritigenin, and those provided in, for example, U.S. Publication Nos. 2009/0012031, 2009/0203010, 2010/0222420, 2011/0251216, 2011/0286990, 2012/0014962, 2012/0071418, 2013/0040906, and 2013/0195843, all of which are incorporated herein by reference; JAK/STAT inhibitors such as lestaurtinib, tofacitinib, ruxolitinib, pacritinib, CYT387, baricitinib, GLPG0636, TG101348, INCB16562, CP-690550, and AZD1480; PKC-β inhibitor such as Enzastaurin; SYK inhibitors such as, but not limited to, GS-9973, R788 (fostamatinib), PRT 062607, R406, (S)-2-(2-((3,5-dimethylphenyl)amino)pyrimidin-4-yl)-N-(1-hydroxypropan-2-yl)-4-methylthiazole-5-carboxamide, R112, GSK143, BAY61-3606, PP2, PRT 060318, R348, and those provided in, for example, U.S. Publication Nos. 2003/0113828, 2003/0158195, 2003/0229090, 2005/0075306, 2005/0232969, 2005/0267059, 2006/0205731, 2006/0247262, 2007/0219152, 2007/0219195, 2008/0114024, 2009/0171089, 2009/0306214, 2010/0048567, 2010/0152159, 2010/0152182, 2010/0316649, 2011/0053897, 2011/0112098, 2011/0245205, 2011/0275655, 2012/0027834, 2012/0093913, 2012/0101275, 2012/0130073, 2012/0142671, 2012/0184526, 2012/0220582, 2012/0277192, 2012/0309735, 2013/0040984, 2013/0090309, 2013/0116260, and 2013/0165431, all of which are incorporated herein by reference; SYK/JAK dual inhibitor such as PRT2070; nitrogen mustards such as bendamustine, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomycins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pralatrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.R™; razoxane; sizofiran; spirogermanium; tenuazonic acid; triaziquone; 2,2′,2″-trichlorotriethyla-mine; urethan; vindesine; dacarbazine; mannomustine; mitobronitol; mitolactol; pipobroman; gacytosine; arabinoside (Ara-C); cyclophosphamide; thiotepa; taxanes, e.g., paclitaxel (e.g., TAXOL™) and docetaxel (e.g., TAXOTERE™) and ABRAXANE® (paclitaxel protein-bound particles); retinoic acid; esperamicins; capecitabine; and pharmaceutically acceptable forms (e.g., pharmaceutically acceptable salts, hydrates, solvates, isomers, prodrugs, and isotopically labeled derivatives) of any of the above. Also included as suitable chemotherapeutic cell conditioners are anti-hormonal agents that act to regulate or inhibit hormone action on tumors such as anti-estrogens including for example tamoxifen (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition as provided herein can be used in combination with commonly prescribed anti-cancer drugs such as Herceptin®, Avastin®, Erbitux®, Rituxan®, Taxol®, Arimidex®, Taxotere®, ABVD, AVICINE, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastic, antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, crizotinib, cell-cycle nonspecific antineoplastic agents, dichloroacetic acid, discodermolide, elsamitrucin, enocitabine, epothilone, eribulin, everolimus, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitor, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

In some embodiments, the chemotherapeutic is selected from hedgehog inhibitors including, but not limited to IPI-926 (See U.S. Pat. No. 7,812,164). Other suitable hedgehog inhibitors include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein. Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354, each incorporated herein by reference. Additional examples of hedgehog inhibitors include, but are not limited to, GDC-0449 (also known as RG3616 or vismodegib) described in, e.g., Von Hoff D. et al., N. Engl. J. Med. 2009; 361(12):1164-72; Robarge K. D. et al., BioorgMed Chem Lett. 2009; 19(19):5576-81; Yauch, R. L. et al. (2009) Science 326: 572-574; Sciencexpress: 1-3 (10.1126/science.1179386); Rudin, C. et al. (2009) New England J of Medicine 361-366 (10.1056/nejma0902903); BMS-833923 (also known as XL139) described in, e.g., in Siu L. et al., J. Clin. Oncol. 2010; 28:15s (suppl; abstr 2501); and National Institute of Health Clinical Trial Identifier No. NCT006701891; LDE-225 described, e.g., in Pan S. et al., ACS Med. Chem. Lett., 2010; 1(3): 130-134; LEQ-506 described, e.g., in National Institute of Health Clinical Trial Identifier No. NCT01106508; PF-04449913 described, e.g., in National Institute of Health Clinical Trial Identifier No. NCT00953758; Hedgehog pathway antagonists disclosed in U.S. Patent Application Publication No. 2010/0286114; SMOi2-17 described, e.g., U.S. Patent Application Publication No. 2010/0093625; SANT-1 and SANT-2 described, e.g., in Rominger C. M. et al., J Pharmacol. Exp. Ther. 2009; 329(3):995-1005; 1-piperazinyl-4-arylphthalazines or analogues thereof, described in Lucas B. S. et al., Bioorg. Med. Chem. Lett. 2010; 20(12):3618-22.

Other hormonal therapy and chemotherapeutic agents include, but are not limited to, anti-estrogens (e.g. tamoxifen, raloxifene, and megestrol acetate), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids or taxanes (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, raltitrexed, tegafur-uracil, capecitabine), cytosine analogs (e.g. cytarabine (ara C, cytosine arabinoside), and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracyclines (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), thalidomide, lenalidomide (REVLIMID®), tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbazine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In some embodiments, a combination of a PI3K inhibitor provided herein (e.g., Compound 1 or CAL-101) and a second agent provided herein, is administered further in combination with an inhibitor of one or more members of TAM family, a receptor tyrosine kinase (RTK) subfamily comprising Tyro-3 (also called Sky), Axl and Mer. In one embodiment, the TAM inhibitor is BGB324 (R428), S49076, TP0903, CEP-40783, ONO-9330547, bosutinib (SKI606, PF5208763), cabozantinib (XL184), sunitinib (SU11248), foretinib (XL880, GSK1363089), MGCD265, BMS777607 (ASLAN002), LY2801653, SGI7079, amuvatinib (SGI-0470-02, MP470), SNS314, PF-02341066, diaminopyrimidine, spiroindoline, UNC569, UNC1062, UNC1666, UNC2025, or LDC1267. Additional TAM inhibitors include those described in Mollard et al., Med. Chem. Lett. 2011, 2, 907-912 and Feneyrolles et al., Mol. Cancer Ther. 13(9), Published OnlineFirst Aug. 19, 2014, the entireties of which are incorporated by reference herein.

### 4. Formulations

The formulations or compositions described herein can include a PI3K inhibitor (e.g., one or more PI3K inhibitors as described herein) and/or one or more additional agents (e.g., a second agent, e.g., one or more second agents) as described herein. In certain embodiments, the PI3K inhibitor (e.g., one or more PI3K inhibitors as described herein) and the second agent are included in the same dosage form. In certain embodiments, the PI3K inhibitor (e.g., one or more PI3K inhibitors as described herein) and the second agent are included in separate dosage forms.

Pharmaceutical compositions may be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue, and intraduodenal routes; parenteral administration, including intravenous, intraarterial, subcutaneous, intramuscular, intravascular, intraperitoneal or infusion as, for example, a sterile solution or suspension, or sustained-release formulation; topical application, for example, as a cream, ointment, or a controlled-release patch or spray applied to the skin; intravaginally or intrarectally, for example, as a pessary, cream, stent or foam; sublingually; ocularly; pulmonarily; local delivery by catheter or stent; intrathecally, or nasally.

The amount of PI3K inhibitor administered and the timing of PI3K inhibitor administration will depend on the type (species, gender, age, weight, etc.) and condition of the patient being treated, the severity of the disease or condition being treated, and on the route of administration. For example, small molecule PI3K inhibitors or second agent can be administered to a patient in doses ranging from 0.001 to 100 mg/kg of body weight per day or per week in single or divided doses, or by continuous infusion. In particular, compounds such as Compound 1, or similar compounds, can be administered to a patient in doses ranging from 5-200 mg per day, or 100-1600 mg per week, in single or divided doses, or by continuous infusion. In one embodiment, the dose is 150 mg/day. Antibody-based PI3K inhibitors or second agent, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds described herein may be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Methods of preparing these formulations or compositions include the step of bringing into association a compound described herein and/or the chemotherapeutic with the carrier and, optionally, one or more accessory ingredients. In general, the formulations are prepared by uniformly and intimately bringing into association a compound as disclosed herein with liquid carriers, or finely divided solid carriers, or both, and then, if necessary, shaping the product.

Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles ofDrug Action, Third Edition, Churchill Livingston, New York, 1990; Katzung, ed., Basic and Clinical Pharmacology, Twelfth Edition, McGraw Hill, 2011; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety. Except insofar as any conventional excipient medium is incompatible with the compounds provided herein, such as by producing any undesirable biological effect or otherwise interacting in a deleterious manner with any other component(s) of the pharmaceutically acceptable composition, the excipient's use is contemplated to be within the scope of this disclosure.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is less than about 100%, about 90%, about 80%, about 70%, about 60%, about 50%, about 40%, about 30%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v or v/v.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is greater than about 90%, about 80%, about 70%, about 60%, about 50%, about 40%, about 30%, about 20%, about 19.75%, about 19.50%, about 19.25%, about 19%, about 18.75%, about 18.50%, about 18.25%, about 18%, about 17.75%, about 17.50%, about 17.25%, about 17%, about 16.75%, about 16.50%, about 16.25%, about 16%, about 15.75%, about 15.50%, about 15.25%, about 15%, about 14.75%, about 14.50%, about 14.25%, about 14%, about 13.75%, about 13.50%, about 13.25%, about 13%, about 12.75%, about 12.50%, about 12.25%, about 12%, about 11.75%, about 11.50%, about 11.25%, about 11%, about 10.75%, about 10.50%, about 10.25%, about 10%, about 9.75%, about 9.50%, about 9.25%, about 9%, about 8.75%, about 8.50%, about 8.25%, about 8%, about 7.75%, about 7.50%, about 7.25%, about 7%, about 6.75%, about 6.50%, about 6.25%, about 6%, about 5.75%, about 5.50%, about 5.25%, about 5%, about 4.75%, about 4.50%, about 4.25%, about 4%, about 3.75%, about 3.50%, about 3.25%, about 3%, about 2.75%, about 2.50%, about 2.25%, about 2%, about 1.75%, about 1.50%, about 1.25%, about 1%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, about 0.009%, about 0.008%, about 0.007%, about 0.006%, about 0.005%, about 0.004%, about 0.003%, about 0.002%, about 0.001%, about 0.0009%, about 0.0008%, about 0.0007%, about 0.0006%, about 0.0005%, about 0.0004%, about 0.0003%, about 0.0002%, or about 0.0001%, w/w, w/v, or v/v.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, or approximately 1% to approximately 10%, w/w, w/v or v/v.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, or approximately 0.1% to approximately 0.9%, w/w, w/v or v/v.

In some embodiments, the concentration of the PI3K inhibitor (e.g., Compound 1) or another agent (e.g., the second agent, e.g., one or more second agents as described herein) provided a pharmaceutical composition disclosed herein or administered in a method disclosed herein is equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the concentration of the PI3K inhibitor (e.g., Compound 1) or another agent, (e.g., the second agent, e.g., one or more second agents as described herein) provided a pharmaceutical composition disclosed herein or administered in a method disclosed herein is more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

In some embodiments, the amount of Compound 1 or one or more of the therapeutic agent disclosed herein is in the range of about 0.0001 to about 10 g, about 0.0005 to about 9 g, about 0.001 to about 8 g, about 0.005 to about 7 g, about 0.01 to about 6 g, about 0.05 to about 5 g, about 0.1 to about 4 g, about 0.5 to about 4 g, or about 1 to about 3 g.

### 4.1 Formulations for Oral Administration

In some embodiments of the methods described herein, PI3K inhibitor (e.g., one or more PI3K inhibitors) and/or another agent (e.g., the second agent, e.g., one or more second agents as described herein) is administered orally. In certain embodiments of the compositions described herein, PI3K inhibitor (e.g., Compound 1) and/or another agent (e.g., the second agent, e.g., one or more second agents as described herein) is formulated for oral administration. Some embodiments pertaining to such methods and compositions include the following.

In some embodiments, provided herein are pharmaceutical compositions for oral administration containing a compound as disclosed herein, and a pharmaceutical excipient suitable for oral administration. In some embodiments, provided herein are pharmaceutical compositions for oral administration containing: (i) an effective amount of a disclosed compound; optionally (ii) an effective amount of one or more second agents; and (iii) one or more pharmaceutical excipients suitable for oral administration. In some embodiments, the pharmaceutical composition further contains: (iv) an effective amount of a third agent.

In some embodiments, the pharmaceutical composition can be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more ingredients. In general, the pharmaceutical compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The present disclosure further encompasses anhydrous pharmaceutical compositions and dosage forms comprising an active ingredient, since water can facilitate the degradation of some compounds. For example, water can be added (e.g., about 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. For example, pharmaceutical compositions and dosage forms which contain lactose can be made anhydrous if substantial contact with moisture and/or humidity during manufacturing, packaging, and/or storage is expected. An anhydrous pharmaceutical composition can be prepared and stored such that its anhydrous nature is maintained. Accordingly, anhydrous pharmaceutical compositions can be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. In some embodiments, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants can be used in the pharmaceutical compositions as provided herein to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein can be combined with various sweetening or flavoring agents, coloring matter or dyes and, for example, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed.

A suitable hydrophilic surfactant can generally have an HLB value of at least about 10, while suitable lipophilic surfactants can generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants can be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants can be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants can include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of glycerides, vegetable oils, hydrogenated vegetable oils, fatty acids and sterols; oil-soluble vitamins/vitamin derivatives; and mixtures thereof. Within this group, non-limiting examples of lipophilic surfactants include glycerol fatty acid esters, propylene glycol fatty acid esters, and mixtures thereof, or are hydrophobic transesterification products of a polyol with at least one member of vegetable oils, hydrogenated vegetable oils, and triglycerides.

In one embodiment, the pharmaceutical composition can include a solubilizer to ensure good solubilization and/or dissolution of a compound as provided herein and to minimize precipitation of the compound. This can be especially important for pharmaceutical compositions for non-oral use, e.g., pharmaceutical compositions for injection. A solubilizer can also be added to increase the solubility of the hydrophilic drug and/or other components, such as surfactants, or to maintain the pharmaceutical composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, s-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers can also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. In some embodiments, solubilizers include sorbitol, glycerol, triacetin, ethyl alcohol, PEG-400, glycofurol and propylene glycol.

The amount of solubilizer that can be included is not particularly limited. The amount of a given solubilizer can be limited to a bioacceptable amount, which can be readily determined by one of skill in the art. In some circumstances, it can be advantageous to include amounts of solubilizers far in excess of bioacceptable amounts, for example to maximize the concentration of the drug, with excess solubilizer removed prior to providing the pharmaceutical composition to a subject using conventional techniques, such as distillation or evaporation. Thus, if present, the solubilizer can be in a weight ratio of about 10%, 25%, 50%, 100%, or up to about 200% by weight, based on the combined weight of the drug, and other excipients. If desired, very small amounts of solubilizer can also be used, such as about 5%, 2%, 1% or even less. Typically, the solubilizer can be present in an amount of about 1% to about 100%, more typically about 5% to about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, oils, odorants, opacifiers, suspending agents, binders, fillers, plasticizers, lubricants, and mixtures thereof.

Exemplary preservatives can include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

In addition, an acid or a base can be incorporated into the pharmaceutical composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Examples can include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### 4.2 Formulations for Parenteral Administration

In some embodiments of the methods described herein, PI3K inhibitor (e.g., one or more PI3K inhibitors) and/or another agent (e.g., the second agent, e.g., one or more second agents as described herein) is administered parenterally. In certain embodiments of the compositions described herein, PI3K inhibitor (e.g., Compound 1) and/or another agent (e.g., the second agent, e.g., one or more second agents as described herein) is formulated for parenteral administration. Some embodiments pertaining to such methods and compositions include the following.

In some embodiments, provided herein are pharmaceutical compositions for parenteral administration containing a compound as disclosed herein, and a pharmaceutical excipient suitable for parenteral administration. In some embodiments, provided herein are pharmaceutical compositions for parenteral administration containing: (i) an effective amount of a disclosed compound; optionally (ii) an effective amount of one or more second agents; and (iii) one or more pharmaceutical excipients suitable for parenteral administration. In some embodiments, the pharmaceutical composition further contains: (iv) an effective amount of a third agent.

The forms in which the disclosed pharmaceutical compositions can be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed.

Aqueous solutions in saline are also conventionally used for injection. Ethanol, glycerol, propylene glycol, liquid polyethylene glycol, and the like (and suitable mixtures thereof), cyclodextrin derivatives, and vegetable oils can also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound as disclosed herein in the required amount in the appropriate solvent with various other ingredients as enumerated above, as appropriate, followed by filtered sterilization. Generally, dispersions are prepared by incorporating the various sterilized active ingredients into a sterile vehicle which contains the basic dispersion medium and the appropriate other ingredients from those enumerated above. In the case of sterile powders for the preparation of sterile injectable solutions, certain methods of preparation are vacuum-drying and freeze-drying techniques which yield a powder of the active ingredient plus any additional ingredient from a previously sterile-filtered solution thereof.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Injectable compositions can contain from about 0.1 to about 5% w/w of a compound as disclosed herein.

### 5. Dosage

The PI3K inhibitor (e.g., Compound 1 or Idelalisib) or another agent disclosed herein (e.g., one or more of the second agents disclosed herein) may be delivered in the form of pharmaceutically acceptable compositions. In certain embodiments, the pharmaceutical compositions comprise the PI3K inhibitor (e.g., Compound 1) described herein and/or one or more additional therapeutic agents, formulated together with one or more pharmaceutically acceptable excipients. In some instances, the PI3K inhibitor (e.g., Compound 1) or one or more of the other therapeutic agents disclosed herein are administered in separate pharmaceutical compositions and may (e.g., because of different physical and/or chemical characteristics) be administered by different routes (e.g., one therapeutic is administered orally, while the other is administered intravenously). In other instances, the PI3K inhibitor (e.g., Compound 1) or one or more of the other therapeutic agents disclosed herein may be administered separately, but via the same route (e.g., both orally or both intravenously). In still other instances, the PI3K inhibitor (e.g., Compound 1) or one or more of the other therapeutic agents disclosed herein may be administered in the same pharmaceutical composition.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of Compound 1 described herein and/or a therapeutic agent will be that amount of the compound which, in some embodiments, may be the lowest dose effective to produce a therapeutic effect. Such an effective dose will generally depend upon the factors described herein. Generally, doses of Compound 1 or the therapeutic agent described herein for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to 1000 mg, or about 0.01 mg to about 500 mg per day, or about 0.1 mg to about 500 mg per day, or about 1 mg to 50 mg per day, or about 5 mg to 40 mg per day. An exemplary dosage is about 10 to 30 mg per day. In some embodiments, for a 70 kg human, a suitable dose would be about 0.05 to about 7 g/day, such as about 0.05 to about 2.5 g/day. Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, the compounds may be administered daily, every other day, three times a week, twice a week, weekly, or bi-weekly. The dosing schedule can include a “drug holiday,” e.g., the drug may be administered for two weeks on, one week off, or three weeks on, one week off, or four weeks on, one week off, etc., or continuously, without a drug holiday. The compounds may be administered orally, intravenously, intraperitoneally, topically, transdermally, intramuscularly, subcutaneously, intranasally, sublingually, or by any other route.

In some embodiments, Compound 1 or the therapeutic agent described herein may be administered in multiple doses. Dosing may be about once, twice, three times, four times, five times, six times, or more than six times per day. Dosing may be about once a month, about once every two weeks, about once a week, or about once every other day. In another embodiment, Compound 1 as disclosed herein and another therapeutic agent are administered together from about once per day to about 6 times per day. In another embodiment, the administration of Compound 1 as provided herein and a therapeutic agent continues for less than about 7 days. In yet another embodiment, the administration continues for more than about 6 days, about 10 days, about 14 days, about 28 days, about two months, about six months, or about one year. In some cases, continuous dosing is achieved and maintained as long as necessary.

Administration of the pharmaceutical compositions as disclosed herein may continue as long as necessary. In some embodiments, an agent as disclosed herein is administered for more than about 1, about 2, about 3, about 4, about 5, about 6, about 7, about 14, or about 28 days. In some embodiments, an agent as disclosed herein is administered for less than about 28, about 14, about 7, about 6, about 5, about 4, about 3, about 2, or about 1 day. In some embodiments, a therapeutic agent as disclosed herein is administered chronically on an ongoing basis, e.g., for the treatment of chronic effects.

Since Compound 1 described herein may be administered in combination with one or more therapeutic agent, the doses of each agent or therapy may be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day.

When Compound 1 is administered in a pharmaceutical composition that comprises one or more therapeutic agents, and the agent has a shorter half-life than Compound 1, unit dose forms of the agent and Compound 1 can be adjusted accordingly.

### 6. Kits

In some embodiments, provided herein are kits. The kits may include a pharmaceutical composition as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information may be based on the results of various studies, for example, studies using experimental animals involving in vivo models and studies based on human clinical trials.

In some embodiments, a memory aid is provided with the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . Second Week, Monday, Tuesday, . . . ” etc. Other variations of memory aids will be readily apparent. A “daily dose” may be a single tablet or capsule or several tablets or capsules to be taken on a given day.

The kit may contain Compound 1 and one or more therapeutic agents. In some embodiments, Compound 1 and the agent are provided as separate pharmaceutical compositions in separate containers within the kit. In some embodiments, Compound 1 as disclosed herein and the agent are provided as a single pharmaceutical composition within a container in the kit. Suitable packaging and additional articles for use (e.g., measuring cup for liquid preparations, foil wrapping to minimize exposure to air, and the like) are known in the art and may be included in the kit. In other embodiments, kits may further comprise devices that are used to administer the active agents. Examples of such devices include, but are not limited to, syringes, drip bags, patches, and inhalers. Kits described herein may be provided, marketed and/or promoted to health providers, including physicians, nurses, pharmacists, formulary officials, and the like. Kits can also, in some embodiments, be marketed directly to the consumer.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules may be removed from the blister pack by manually applying pressure on the recesses whereby an opening is formed in the sheet at the place of the recess. The tablet or capsule can then be removed via said opening.

Kits may further comprise pharmaceutically acceptable vehicles that may be used to administer one or more active agents. For example, if an active agent is provided in a solid form that must be reconstituted for parenteral administration, the kit can comprise a sealed container of a suitable vehicle in which the active agent may be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The present disclosure further encompasses anhydrous pharmaceutical compositions and dosage forms comprising an active ingredient, since water can facilitate the degradation of some compounds. For example, water may be added (e.g., about 5%) in the pharmaceutical arts as a means of simulating long-term storage in order to determine characteristics such as shelf-life or the stability of formulations over time. Anhydrous pharmaceutical compositions and dosage forms may be prepared using anhydrous or low moisture containing ingredients and low moisture or low humidity conditions. For example, pharmaceutical compositions and dosage forms which contain lactose may be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous pharmaceutical compositions may be packaged using materials known to prevent exposure to water such that they may be included in suitable formulary kits. Examples of suitable packaging include, but are not limited to, hermetically sealed foils, plastic or the like, unit dose containers, blister packs, and strip packs.

### EXAMPLES

### Example 1: Combination Study of Compound 1 with Selinexor

The synergistic effects of compounds provided herein and another therapeutic agent were carried out. The method is described as follows. Cells were thawed from a liquid nitrogen preserved state. Once cells were expanded and divided at their expected doubling times, screening began. Cells were seeded in growth media in either black 1536-well or 384-well tissue culture treated plates. Cells were then equilibrated in assay plates via centrifugation and placed in incubators attached to the Dosing Modules at 37° C. for 24 hours before treatment. At the time of treatment, a set of assay plates (which do not receive treatment) were collected and ATP levels were measured by adding ATPLite (Perkin Elmer). These Tzero (T₀) plates were read using ultra-sensitive luminescence on Envision plate readers (Perkin Elmer). Treated assay plates were incubated with compound for 72 hours. After 72 hours, plates were developed for endpoint analysis using ATPLite. All data points were collected via automated processes, quality controlled and analyzed using Zalicus software. Assay plates were accepted if they passed the following quality control standards: relative luciferase values were consistent throughout the entire experiment, Z-factor scores were greater than 0.6, untreated/vehicle controls behaved consistently on the plate.

Inhibition (I) is defined as

$$I = ( 1 - T / V ) * 100 \%$$

where T is treated cell count and V is untreated (vehicle) cell count (at 72 hours). I ranges from 0% (when T=V) to 100% (when T=0). The IC₅₀ value is defined as the drug concentration needed to inhibit 50% of the cell growth compared to growth of the vehicle treated cells (the drug concentration which gives I=50%). The measure of effect in the experiment can be the inhibition of cellular response relative to the untreated level (vehicle alone). For untreated vehicle and treated levels V and T, a fractional inhibition I=1-TV is calculated. The inhibition ranges from 0% at the untreated level to 100% when T=0. Inhibition levels are negative for agents that actually increase levels. Other effect measures, such as an activity ratio r=TV may be more appropriate for some assays. When activity ratios (e.g, fold increase over stimulated control) are being used, the effect can be measured using an induction I=1n(T/V). With this definition, all effect expressions are the same as for inhibition.

Growth Inhibition (GI) is used as a measure of cell viability. The cell viability of vehicle is measured at the time of dosing (TO) and after 72 hours (T72). A GI reading of 0% represents no growth inhibition—T72 compound-treated and T72 vehicle signals are matched. A GI reading of 100% represents complete growth inhibition—T72 compound-treated and TO vehicle signals are matched. Cell numbers have not increased during the treatment period in wells with GI 100% and may suggest a cytostatic effect for compounds reaching a plateau at this effect level. A GI reading of 200% represents complete death of all cells in the culture well. Compounds reaching an activity plateau of GI 200% are considered cytotoxic. GI is calculated by applying the following test and equation:

$$If T < {V}_{0} : 100 * ( 1 - \frac{T - {V}_{0}}{{V}_{0}} )$$

$$If T \geq {V}_{0} : 100 * ( 1 - \frac{T - {V}_{0}}{{V}_{0}} )$$

where T is the signal measure for a test article, V is the vehicle-treated control measure, and Vo is the vehicle control measure at time zero. This formula is derived from the Growth Inhibition calculation used in the National Cancer Institute's NCI-60 high-throughput screen.

Combination analysis data were collected in a 9×9 dose matrix. Synergy was calculated by comparing a combination's response to those of its single compound, against the drug-with-itself dose-additive reference model. Deviations from dose additivity may be assessed visually on an isobologram or numerically with a Combination Index (CI). See the tables below for CI at 50% inhibition and CI at 50% growth inhibition. Additive effect is CI=1.0. Synergistic effect is CI<1. Antagonistic effect is CI >1.0.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This was done by finding the crossing point for each single agent concentration in a dose matrix across the concentrations of the other single agent. Practically, each vertical concentration C_{Y} was held fixed while a bisection algorithm was used to identify the horizontal concentration C_{X} in combination with that vertical dose that gave the chosen effect level in the response surface Z(C_{X}, C_{Y}). These concentrations were then connected by linear interpolation to generate the isobologram display. For synergistic interactions, the isobologram contour would fall below the additivity threshold and approache the origin, and an antagonistic interaction would lie above the additivity threshold. The error bars represented the uncertainty arising from the individual data points used to generate the isobologram. The uncertainty for each crossing point was estimated from the response errors using bisection to find the concentrations where Z-oz(C_{X}, C_{Y}) and Z+σ_{z}(C_{X}, C_{Y}) cross I_{cut}, where σ_{z} is the standard deviation of the residual error on the effect scale.

To measure combination effects in excess of Loewe additivity, a scalar measure to characterize the strength of synergistic interaction termed the Synergy Score was devised. The Synergy Score was calculated as:

$$Synergy Score = log {f}_{X} log {f}_{Y} Σ max ( 0 , {I}_{d a t a} ) ( {I}_{d a t a} - {I}_{L o e w e} )$$

The fractional inhibition for each component agent and combination point in the matrix was calculated relative to the median of all vehicle-treated control wells. The Synergy Score equation integrated the experimentally-observed activity volume at each point in the matrix in excess of a model surface numerically derived from the activity of the component agents using the Loewe model for additivity. Additional terms in the Synergy Score equation (above) were used to normalize for various dilution factors used for individual agents and to allow for comparison of synergy scores across an entire experiment. The inclusion of positive inhibition gating or an I_{data} multiplier removed noise near the zero effect level, and biases results for synergistic interactions at that occur at high activity levels.

The Synergy Score measure was used for the self-cross analysis. Synergy Scores of self-crosses were expected to be additive by definition and, therefore, maintain a synergy score of zero. However, while some self-cross synergy scores were near zero, many were greater suggesting that experimental noise or non-optimal curve fitting of the single agent dose responses were contributing to the slight perturbations in the score. This strategy was cell line-centric, focusing on self-cross behavior in each cell line versus a global review of cell line panel activity. Combinations where the synergy score was greater than the mean self-cross plus two standard deviations or three standard deviations can be considered candidate synergies at 95% and 99% confidence levels, respectively. Additivity should maintain a synergy score of zero, and synergy score of two or three standard deviations indicate that the combination is synergistic at statistically significant levels of 95% and 99%.

Loewe Volume (Loewe Vol) was used to assess the overall magnitude of the combination interaction in excess of the Loewe additivity model. Loewe Volume was particularly useful when distinguishing synergistic increases in a phenotypic activity (positive Loewe Volume) versus synergistic antagonisms (negative Loewe Volume). When antagonisms is observed, the Loewe Volume should be assessed to examine if there is any correlation between antagonism and a particular drug target-activity or cellular genotype. This model defined additivity as a non-synergistic combination interaction where the combination dose matrix surface should be indistinguishable from either drug crossed with itself. The calculation for Loewe additivity is:

$${I}_{L o e w e} that satisfies ( X / {X}_{I} ) + ( Y / {Y}_{I} ) = 1$$

where X₁ and Y₁ are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

### Results

The CI₅₀ values for growth inhibition and inhibition in Table 1 are categorized as follows: S=0.01 to <0.5, T=0.5 to <0.7, U=0.7 to <1, and W=>1. The synergy score values for growth inhibition and inhibition are categorized as follows: A1=0.0001 to <1, A2=1 to <3, and A3=>3.

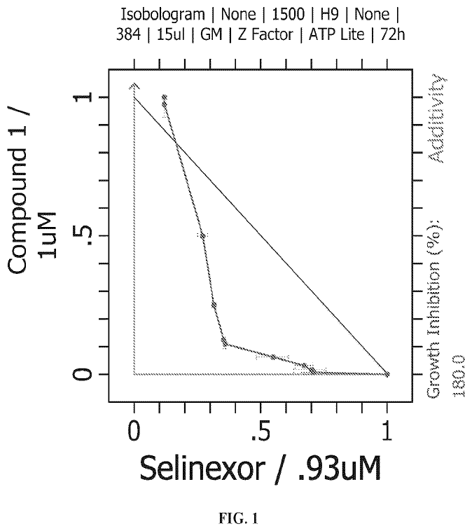

The combination effects of Compound 1 and selinexor were tested in five types of T-cell lymphoma cell line: H9, HH, HuT 78, HuT 102, and MJ (G11). These cell lines may have different genomic profiles and thus, a combination of Compound 1 and selinexor can have different synergistic effects on these cell lines. The results are shown in Table 1 below. An isobologram depicting the effect of the combination of Compound 1 and selinexor in H9 cell line is provided in FIG. 1. The data show that the combination of Compound 1 and selinexor is synergistic in selected cell lines.

TABLE 1
Combination of Compound 1 and Selinexor

| | | Synergy | | | |
|---|---|---|---|---|---|
| Compound 1 in | | Score | CI₅₀ | Synergy | |
| combination | | growth | growth | Score | CI₅₀ |
| with | Cell Line | inhibition | inhibition | inhibition | inhibition |
| Selinexor | H9 | 10.4 A3 | | 3.23 A3 | |
| Selinexor | HH | 1.53 A2 | | 1.25 A2 | |
| Selinexor | HuT 102 | | | 0.82 A1 | |
| Selinexor | HuT 78 | 5.85 A3 | | 1.94 A2 | |
| Selinexor | MJ (G11) | 4.50 A3 | | 1.50 A2 | |

### Example 2: Clinical Trial for Compound 1 and Anti-PD-1 Antibody Combination

A phase 1b clinical trial for treatment of patient with hematological malignancies with combination of Compound 1 and anti-PD-1 antibody is carried out. Some patients have advanced B and/or T cell maliganceis. The anti-PD-1 antibodies used in this study in combination with Compound 1 include Nivolumab and Pembrolizumab.

The starting dose for Compound 1 is 15 mg QD, and may be escalated to 15 mg BID, 25 mg BID, and 25 mg QD.

The combination of Compound 1 and the anti-PD-1 antibody are administered to three expansion cohorts: follicular lymphoma, DLBCL, and T-cell lymphoma.

### EQUIVALENTS

While this invention has been disclosed with reference to specific aspects, it is apparent that other aspects and variations of this invention can be devised by others skilled in the art without departing from the true spirit and scope of the invention. The appended claims are intended to be construed to include all such aspects and equivalent variations.

What is claimed is:

1. A method of treating, managing, or preventing a cancer in a subject comprising administering to the subject a therapeutically effective amount of a PI3K inhibitor in combination with a second therapeutic agent, wherein the second therapeutic agent is 1) a checkpoint modulator, 2) an XPO1 inhibitor, 3) an anti-CD19 antibody, 4) a TLR agonist, 5) a STING agonist, or 6) a Flt3 ligand, or a combination thereof.

2. The method of claim 1, wherein the PI3K-inhibitor is a PI3K delta/gamma dual inhibitor.

3. The method of claim 1, wherein the PI3K-inhibitor is Compound 1 of the following structure: 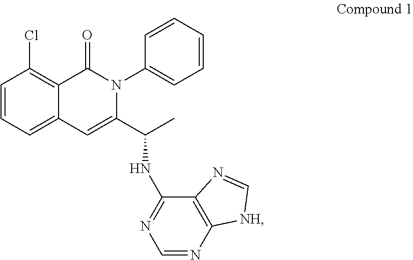 [SMILES: C[C@H](Nc1ncnc2[nH]cnc12)c1cc2cccc(Cl)c2c(=O)n1-c1ccccc1]
or a pharmaceutically acceptable form thereof.

4. The method of claim 1, wherein the PI3K-inhibitor is a PI3K delta inhibitor.

5. The method of claim 1, wherein the PI3K-inhibitor is Idelalisib of the following structure: 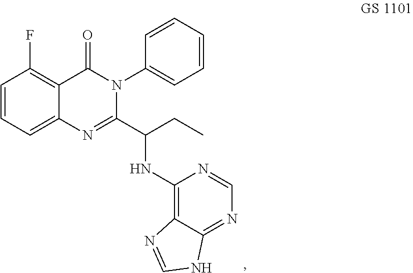 [SMILES: CCC(Nc1ncnc2[nH]cnc12)c1nc2cccc(F)c2c(=O)n1-c1ccccc1]
or a pharmaceutically acceptable form thereof.

6. The method of any one of claims 1 to 5, wherein second therapeutic agent is a checkpoint modulator.

7. The method of claim 6, wherein the checkpoint modulator is a modulator of CTLA-4, CD80, CD86, PD-1, PD-L1, PD-L2, LAG-3, Galectin-3, BTLA, TIM3, GAL9, B7-H1, B7-H3, B7-H4, TIGIT/Vstm3/WUCAM/VSIG9, VISTA, GITR, HVEM, OX40, CD27, CD28, CD137. CGEN-15001T, CGEN-15022, CGEN-15027, CGEN-15049, CGEN-15052, or CGEN-15092.

8. The method of claim 6, wherein the checkpoint modulator is a PD-1 inhibitor.

9. The method of claim 6, wherein the checkpoint modulator is an anti-PD-1 antibody.

10. The method of claim 9, wherein the anti-PD-1 antibody is Nivolumab, Pembrolizumab, Pidilizumab, AMP-514, or AMP-224, or a combination thereof.

11. The method of claim 10, wherein the anti-PD-1 antibody is Nivolumab.

12. The method of claim 10, wherein the anti-PD-1 antibody is Pembrolizumab.

13. The method of claim 6, wherein the checkpoint modulator is an anti-PD-L1 antibody.

14. The method of claim 13, wherein the anti-PD-L1 antibody is MDX-1105, YW243.55.S70, MDPL3280A, MSB0010718C, or durvalumab, or a combination thereof.

15. The method of claim 6, wherein the checkpoint modulator is an anti-CTLA-4 antibody.

16. The method of claim 15, wherein the an anti-CTLA-4 antibody is Tremelimumab or Ipilimumab, or a combination thereof.

17. The method of claim 16, wherein the an anti-CTLA-4 antibody is Tremelimumab.

18. The method of claim 16, wherein the an anti-CTLA-4 antibody is Ipilimumab.

19. The method of claim 6, wherein the checkpoint modulator is a LAG-3 inhibitor.

20. The method of claim 6, wherein the checkpoint modulator is a TIM3 inhibitor.

21. The method of claim 6, wherein the checkpoint modulator is a B7 inhibitor.

22. The method of claim 6, wherein the checkpoint modulator is an IDO inhibitor.

23. The method of claim 6, wherein the checkpoint modulator is an agonist of OX40.

24. The method of any one of claims 1 to 5, wherein second therapeutic agent is an XPO1 inhibitor.

25. The method of claim 24, wherein the XPO1 inhibitor is selinexor, KPT-251, KPT-276, or SL-801, or a combination thereof.

26. The method of claim 25, wherein the XPO1 inhibitor is selinexor.

27. The method of any one of claims 1 to 5, wherein second therapeutic agent is an anti-CD19 antibody.

28. The method of claim 27, wherein the anti-CD19 antibody is blinatumomab.

29. The method of any one of claims 1 to 5, wherein second therapeutic agent is a TLR agonist.

30. The method of any one of claims 1 to 5, wherein second therapeutic agent is a STING agonist.

31. The method of any one of claims 1 to 5, wherein second therapeutic agent is a Flt3 ligand.

32. The method of any one of claims 1 to 31, wherein the PI3K inhibitor and the second therapeutic agent are the only therapeutically active ingredients.

33. The method of any one of claims 1 to 32, wherein the PI3K inhibitor and the second therapeutic agent are in a single dosage form.

34. The method of any one of claims 1 to 32, wherein the PI3K inhibitor and the second therapeutic agent are in separate dosage forms.

35. The method of any one of claims 1 to 34, wherein the combination of the PI3K inhibitor and the second therapeutic agent is synergistic in treating a cancer.

36. The method of any one of claims 1 to 35, wherein the concentration of the PI3K inhibitor that is required to achieve a level of inhibition is at least 20% lower when the PI3K inhibitor is administered in combination with the second therapeutic agent than when the PI3K inhibitor is administered alone.

37. The method of any one of claims 1 to 36, wherein the concentration of the second therapeutic agent that is required to achieve a level of inhibition is at least 20% lower when the second therapeutic agent is administered in combination with PI3K inhibitor than when the second therapeutic agent is administered alone.

38. The method of any one of claims 1 to 37, wherein the dose of the PI3K inhibitor that achieves a therapeutic effect is at least 20% lower when the PI3K inhibitor is administered in combination with the second therapeutic agent than when the PI3K inhibitor is administered alone.

39. The method of any one of claims 1 to 38, wherein the dose of the second therapeutic agent that achieves a therapeutic effect is at least 20% lower when the second therapeutic agent is administered in combination with PI3K inhibitor than when the second therapeutic agent is administered alone.

40. The method of any one of claims 1 to 39, wherein the anti-cancer effect provided by the combination of the PI3K inhibitor and the second therapeutic agent is greater than the anti-cancer effect provided by a monotherapy with the same dose of the PI3K inhibitor.

41. The method of claim 40, wherein the anti-cancer effect provided by the combination of the PI3K inhibitor and the second therapeutic agent is at least 2 fold greater, at least 3 fold greater, at least 5 fold greater, or at least 10 fold greater than the anti-cancer effect provided by the monotherapy with the PI3K inhibitor or pharmaceutically acceptable form thereof.

42. The method of any one of claims 1 to 41, wherein the anti-cancer effect provided by the combination of the PI3K inhibitor and the second therapeutic agent is greater than the anti-cancer effect provided by a monotherapy with the same dose of the second therapeutic agent.

43. The method of claim 42, wherein the anti-cancer effect provided by the combination of the PI3K inhibitor and the second therapeutic agent is at least 2 fold greater, at least 3 fold greater, at least 5 fold greater, or at least 10 fold greater than the anti-cancer effect provided by the monotherapy with the second therapeutic agent.

44. The method of any one of claims 1 to 43, wherein the PI3K inhibitor is administered concurrently with the second therapeutic agent.

45. The method of any one of claims 1 to 43, wherein the PI3K inhibitor is administered subsequent to the second therapeutic agent.

46. The method of any one of claims 1 to 43, wherein the PI3K inhibitor is administered prior to the second therapeutic agent.

47. The method of any one of claims 1 to 46, wherein resistance of the cancer to the PI3K inhibitor is delayed.

48. The method of any one of claims 1 to 46, wherein the risk that the cancer becomes resistant to the PI3K inhibitor is reduced.

49. The method of any one of claims 1 to 46, wherein the cancer does not become resistant to the PI3K inhibitor for at least 12 months.

50. The method of any one of claims 1 to 46, wherein remission of the cancer in the subject is prolonged.

51. The method of any one of claims 1 to 46, wherein the subject experiences remission of the cancer for at least 12, 18, or 24 months.

52. The method of any one of claims 1 to 46, wherein the likelihood that the subject experiences complete remission of the cancer is increased.

53. The method of any one of claims 1 to 46, wherein the subject experiences complete remission of the cancer.

54. The method of any one of claims 1 to 46, wherein the level of minimal residual disease (MRD) is reduced.

55. The method of any one of claims 1 to 46, wherein the subject has substantially no detectable MRD after the treatment.

56. The method of any one of claims 1 to 55, wherein the combination of the PI3K inhibitor and the second therapeutic agent is synergistic as indicated by a combination index value that is less than 1.

57. The method of claim 56, wherein the combination of the PI3K inhibitor and the second therapeutic agent is synergistic as indicated by a combination index value that is less than 0.7.

58. The method of claim 57, wherein the combination of the PI3K inhibitor and the second therapeutic agent is synergistic as indicated by a combination index value that is less than 0.5.

59. The method of any one of claims 56 to 58, wherein the combination index value is assessed at 50% inhibition.

60. The method of any one of claims 56 to 58, wherein the combination index value is assessed at 50% growth inhibition.

61. The method of any one of claims 1 to 55, wherein the combination of the PI3K inhibitor and the second therapeutic agent is synergistic as indicated by a synergy score value of greater than 3.

62. The method of claim 61, wherein the combination of the PI3K inhibitor and the second therapeutic agent is synergistic as indicated by a synergy score value of greater than 3 for inhibition or growth inhibition.

63. A method of reducing the likelihood for a subject to develop resistance to a treatment with a PI3K inhibitor, comprising:
(a) administering to the subject a therapeutically effective amount of a monotherapy comprising the PI3K inhibitor for a first period of time;
(b) after the first period of time, administering to the subject a therapeutically effective amount of a combination therapy comprising the PI3K inhibitor in combination with 1) a checkpoint modulator, 2) an XPO1 inhibitor, 3) an anti-CD19 antibody, 4) a TLR agonist, 5) a STING agonist, or 6) a Flt3 ligand, or a combination thereof, for a second period of time; and
(c) optionally repeating steps (a) and (b) one or more times.

64. A method of delaying or decreasing resistance of a subject having a cancer, comprising administering to the subject a therapeutically effective amount of a PI3K inhibitor in combination with a second therapeutic agent, wherein the second therapeutic agent is 1) a checkpoint modulator, 2) an XPO1 inhibitor, 3) an anti-CD19 antibody, 4) a TLR agonist, 5) a STING agonist, or 6) a Flt3 ligand, or a combination thereof, thereby delaying or decreasing resistance.

65. The method of claim 64, wherein the resistance is resistance to the PI3K inhibitor.

66. The method of claim 64 or 65, wherein the PI3K inhibitor is administered prior to the second therapeutic agent.

67. A method of reducing the level of minimal residual disease (MRD) in a subject having a cancer, comprising administering to the subject a therapeutically effective amount of a PI3K inhibitor in combination with a second therapeutic agent, wherein the second therapeutic agent is 1) a checkpoint modulator, 2) an XPO1 inhibitor, 3) an anti-CD19 antibody, 4) a TLR agonist, 5) a STING agonist, or 6) a Flt3 ligand, or a combination thereof, thereby reducing the level of MRD in the subject.

68. The method of any one of claims 63 to 67, wherein the PI3K inhibitor is Compound 1 of the following formula: 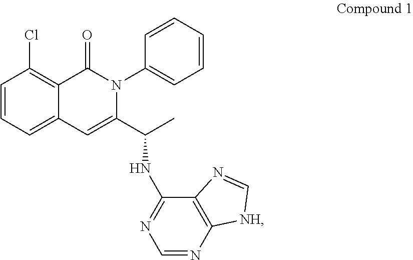 [SMILES: C[C@H](Nc1ncnc2[nH]cnc12)c1cc2cccc(Cl)c2c(=O)n1-c1ccccc1]
or a pharmaceutically acceptable form thereof.

69. The method of any one of claims 1 to 68, wherein the cancer is of hematopoietic origin.

70. The method of claim 69, wherein the cancer is lymphoma or leukemia.

71. The method of claim 69, wherein the cancer is B-cell lymphoma, mantle cell lymphoma, non-Hodgkin's B-cell lymphoma, non-Hodgkin's lymphoma T-cell lymphoma, cutaneous lymphoma, anaplastic large cell lymphoma, multiple myeloma, myeloma, or plasmacytoma.

72. The method of claim 69, wherein the cancer is multiple myeloma.

73. The method of claim 69, wherein the cancer is non-Hodgkin's lymphoma.

74. The method of claim 73, wherein the non-Hodgkin's lymphoma is B cell non-Hodgkin's lymphoma.

75. The method of claim 74, wherein the B cell non-Hodgkin's lymphoma is diffuse large B-cell lymphoma.

76. The method of claim 75, wherein the diffuse large B-cell lymphoma is diffuse large B-cell lymphoma activated B-cell like or diffuse large B-cell lymphoma germinal center B-cell-like.

77. The method of claim 69, wherein the cancer is indolent non-Hodgkin's lymphoma.

78. The method of claim 69, wherein the cancer is follicular lymphoma.

79. The method of claim 69, wherein the cancer is mantle cell lymphoma.

80. The method of claim 69, wherein the cancer is T-cell lymphoma.

81. The method of any one of claims 1 to 80, wherein the subject is a human.

82. A composition comprising a combination of a PI3K inhibitor and a second therapeutic agent, wherein the second therapeutic agent is 1) a checkpoint modulator, 2) an XPO1 inhibitor, 3) an anti-CD19 antibody, 4) a TLR agonist, 5) a STING agonist, or 6) a Flt3 ligand, or a combination thereof.
